(19) United States
(12) Patent Application Publication
(10) Pub. No.: US 2025/0009742 A1
(43) Pub. Date: Jan. 9, 2025

(54) NOVEL CAPSID ASSEMBLY INHIBITORS

(71) Applicants: AM SCIENCES INC, Seoul (KR); KOREA RESEARCH INSTITUTE OF CHEMICAL TECHNOLOGY, Daejeon (KR)

(72) Inventors: Nakcheol JEONG, Seoul (KR); Misun LEE, Seoul (KR); Heewoo SIM, Seoul (KR); Myungjin KIM, Seoul (KR); Jung-Hee KIM, Seoul (KR); Soo Bong HAN, Daejeon (KR); Young Sik JUNG, Daejeon (KR); Hye jin KIM, Daejeon (KR); Chang Soo YUN, Daejeon (KR); Heeyeong CHO, Daejeon (KR); Mee Hyein KIM, Daejeon (KR); Joo Youn LEE, Daejeon (KR)

(21) Appl. No.: 18/699,156

(22) PCT Filed: Oct. 12, 2022
(86) PCT No.: PCT/KR2022/015333

§ 371 Date: Apr. 5, 2024

(30) Foreign Application Priority Data
Oct. 12, 2021 (KR) 10-2021-0134939

(51) Int. Cl. A61K 31/501; A61K 31/4155; A61K 31/4162; A61K 31/422; A61K 31/437; A61K 31/4439; A61K 31/506; A61P 31/20; C07D 231/12; C07D 231/16; C07D 231/56; C07D 401/12; C07D 401/14; C07D 403/12; C07D 403/14; C07D 405/14; C07D 471/04; C07D 491/052; C07D 491/056; C07D 495/04

(52) U.S. Cl. CPC A61K 31/501; A61K 31/4155; A61K 31/4162; A61K 31/422; A61K 31/437; A61K 31/4439; A61K 31/506; A61P 31/20; C07D 231/12; C07D 231/16; C07D 231/56; C07D 401/12; C07D 401/14; C07D 403/12; C07D 403/14; C07D 405/14; C07D 471/04; C07D 491/052; C07D 491/056; C07D 495/04

(57) ABSTRACT

The present invention relates to a series of novel pyrazolylmethylurea derivatives, and the use thereof for the inhibition of capsid assembly and the prevention or treatment of viral diseases through the inhibition.

### TECHNICAL FIELD

The present invention relates to a series of novel pyrazolylmethylurea derivatives, and the use thereof for the inhibition of capsid assembly and the prevention or treatment of viral diseases through the inhibition.

### BACKGROUND ART

Chronic hepatitis B virus (HBV) infection is a major health problem worldwide and may cause serious health problems such as cirrhosis or liver cancer. According to a recent WHO report, it is estimated that 257 million people worldwide are living with chronic HBV infection and 1.34 million people die each year due to hepatitis-related complications. To date, substances approved for the treatment of HBV include interferons (IFNs, non-pegylated or pegylated) and nucleos(t)ide analogues, lamivudine, adefovir, entecavir, tenofovir and the like. IFN therapy induces suppression of HBV replication and remission of liver disease, and nucleos(t)ide drugs suppress reverse transcriptase and DNA polymerase activity. Nucleos(t)ide analogues effectively control viral proliferation, but the viral gene, which is the key to HBV, remains together in the form of cccDNA as in the form of the host's mini-chromosome, and thus complete elimination thereof is difficult. Therefore, drug resistance occurs frequently in HBV carriers because they should use drugs for a long time to prevent the growth of new virus. In order to overcome this unmet medical need, there is a need for the discovery of efficient and safe anti-HBV drugs with novel molecular targets.

The HBV core proteins play an important role in the viral life cycle. The HBV capsid formed by the assembly of core proteins encapsulates pregenomic RNA (pgRNA), reverse transcriptase, and DNA polymerase together to modulate reverse transcription of pgRNA and recycling of nucleocapsids. The core proteins modulate the transport and nuclear release of viral genome, are involved in epigenetic regulation of cccDNA to modulate host gene expression. Hence, based on the HBV replication cycle, the modulation of the action of core proteins and the discovery of target anti-HBV agents, which prevent the formation of capsid proteins composed of core proteins, have been extensively studied.

A number of research centers and pharmaceutical companies have developed capsid assembly modulators. Bay-41-4109, a heteroaryldihydropyrimidine (HAP) analog, is the first capsid assembly inhibitor to enter clinical trials, and induces aberrant formation of capsids and aggregation of capsid proteins. As a result of incorrect assembly of capsid proteins, it was observed in HepG2.2.15 cells that HBV core protein was degraded by proteasome (proteasome mediated degradation) together with HBV DNA reduction by inhibiting HBV DNA replication. GLS-4 is a secondgeneration HAP analog having the same mechanism of action as BAY-41-4109, and is in phase II clinical trials together with ritonavir (RTV) in order to prevent the induction of CYP enzymes by GLS-4. The same researchers have reported HEC72702 with reduced CYP enzyme induction, lower suppression of hERG K⁺ channels, and improved oral bioavailability while sacrificing some in vitro potency.

Other types of capsid assembly modulators, AT130, NVR 3-778, and JNJ-632, with a different mechanism of action from that of HAPs, have also been reported. For example, when capsid is treated with NVR 3-778, capsid is normally formed, but an empty capsid into which pregenomic RNA (pgRNA), reverse transcriptase, and DNA polymerase are not included is obtained. JNJ-632 has been reported as a novel sulfamoylbenzamide capsid assembly modulator, and its optimized analog JNJ-6379 is currently in phase II clinical trials. Recently, GIST researchers Kang, J. A. et al. have reported ciclopirox, an FDA-approved antifungal drug, as an orally available capsid assembly inhibitor in a drug repositioning strategy.

### CITATION LIST

### Patent Documents

WO 2017/210545; and
US 2017/0355708

### Non-Patent Document

Kang, J. A. et al., Nat. Commun., 2019, 10(2184):1-14

### DISCLOSURE OF INVENTION

### Technical Problem

As a result of intensive research efforts to discover novel small molecule compounds that can suppress viral infection by inhibiting capsid assembly, the present inventors have confirmed that a series of pyrazolylmethylurea derivatives have the activity of suppressing viral infection by potentially inhibiting capsid assembly, thus completing the present invention.

### Solution to Problem

An object of the present invention is to provide a compound represented by the following Chemical Formula 1 or a pharmaceutically acceptable salt of the compound:

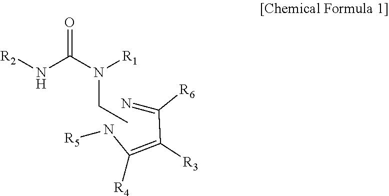

in Chemical Formula 1,
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently a bond, hydrogen, cyano, halogen, C₁₋₄ alkyl, C₁₋₄ alkenyl, C₁₋₄ haloalkyl, C₁₋₄ hydroxyalkyl, C₁₋₄ alkoxy, C₁₋₃ acyl, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including carbon to which R₃ and R₄ are bonded;
Chemical Formula 1 is connected to a urea backbone through either of R₃ or R₆; and
here, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, and 5- to 10-membered heteroaryl.

Another object of the present invention is to provide a method for preparing the compound described above or a pharmaceutically acceptable salt thereof, which includes a first step of reacting a pyrazolylmethyl precursor with an R₁ precursor; and a second step of reacting the compound obtained in the previous step with an R₂ precursor.

Still another object of the present invention is to provide a composition for capsid assembly inhibition, containing the compound described above or a pharmaceutically acceptable salt thereof.

Still another object of the present invention is to provide an antiviral composition containing the compound described above or a pharmaceutically acceptable salt thereof as an active ingredient.

Still another object of the present invention is to provide a pharmaceutical composition for prevention or treatment of viral disease, containing the compound described above or a pharmaceutically acceptable salt thereof as an active ingredient.

Still another object of the present invention is to provide a method for treating a viral disease, which includes administering the pharmaceutical composition described above to an individual in need thereof.

### Advantageous Effects of Invention

The pyrazolylmethylurea derivatives in the molecule newly synthesized according to the present invention exhibit low cytotoxicity and an effect of inhibiting capsid assembly, and can be thus usefully used to prevent or treat diseases related to capsid assembly, for example, viral diseases caused by HBV, HCV, HIV, and the like.

### BEST MODE FOR CARRYING OUT THE INVENTION

Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description below.

Further, those skilled in the art will recognize, or be able to ascertain using no more than routine experimentation, many equivalents to the specific embodiments of the invention described herein. Further, these equivalents should be interpreted to fall within the scope of the present invention.

In addition, throughout this specification, when a part is referred to as “including” an element, it will be understood that other elements may be further included rather than other elements being excluded unless content to the contrary is specially described.

Hereinafter, the present invention will be described in detail.

A first aspect of the present invention provides a compound represented by the following Chemical Formula 1 or a pharmaceutically acceptable salt of the compound:

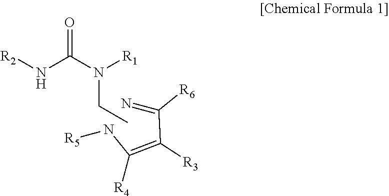

in Chemical Formula 1,
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently a bond, hydrogen, cyano, halogen, C₁₋₄ alkyl, C₁₋₄ alkenyl, C₁₋₄ haloalkyl, C₁₋₄ hydroxyalkyl, C₁₋₄ alkoxy, C₁₋₃ acyl, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including carbon to which R₃ and R₄ are bonded;
Chemical Formula 1 is connected to a urea backbone through either of R₃ or R₆; and
here, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, and 5- to 10-membered heteroaryl.

For example, in Chemical Formula 1, R₁ may be phenyl, bicyclo[1.1.1]pentyl, dihydropyridinyl, isoxazolyl, pyrazolyl, pyridazinyl, pyridinyl, pyrimidinyl, or thiadiazolyl, but is not limited thereto.

For example, in Chemical Formula 1, R₂ may be phenyl, bicyclo[1.1.1]pentyl, imidazolyl, pyridinyl, or pyrimidinyl, but is not limited thereto.

For example, in Chemical Formula 1, R₃ may be hydrogen, bromo, methyl, propenyl, trifluoromethyl, hydroxymethyl, hydroxyethyl, hydroxyisopropyl, hydroxypropyl, hydroxyisobutyl, methoxy, methoxyethyl, methoxymethyl, ethoxyethyl, ethoxyethenyl, or (4,4,5,5-tetramethyl-1,3-dioxolanyl)methyl, but is not limited thereto.

For example, in Chemical Formula 1, R₄ may be a bond, hydrogen, cyano, methyl, ethyl, isopropyl, difluoromethyl, trifluoromethyl, or methoxy, but is not limited thereto.

For example, in Chemical Formula 1, R₃ and R₄ may be connected to each other to form tetrahydrothiopyranyl, tetrahydropyranyl, tetrahydrofuranyl, piperidinyl, phenyl, dioxanyl, cyclopentyl, cyclohexyl, cycloheptyl, or bicyclo[2.2.1]heptyl including carbon to which R₃ and R₄ are bonded, but is not limited thereto.

For example, in Chemical Formula 1, R₅ may be hydrogen, methyl, hydroxymethyl, hydroxyethyl, ethylcarbonyl, or tetrahydropyranyloxyethyl, or pyrimidinyl, but is not limited thereto.

For example, in Chemical Formula 1, R₆ may be a bond or methyl, but is not limited thereto.

For example, in Chemical Formula 1, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other may be unsubstituted or substituted with one or more selected from the group consisting of cyano, hydroxy, carboxyl, oxo, bromo, fluoro, chloro, methyl, methylthio, methoxy, methoxymethyl, methylcarbonyl, methoxycarbonyl, methylsulfonylamino, methylaminosulfonyl, hydroxymethyl, hydroxyisopropyl, difluoromethyl, trifluoromethyl, methylamino, and dimethylamino, but is not limited thereto. [66]

Specifically, the compound may be
1. 3-(bicyclo[1.1.1]pentan-1-yl)-1-(2-methoxypyrimidin-5-yl)-1-((5-(trifluoromethyl)-1H-pyrazol-3-yl)methyl)urea,
2. 1-(2-methoxypyrimidin-5-yl)-3-(pyrimidin-5-yl)-1-((5-(trifluoromethyl)-1H-pyrazol-3-yl)methyl)urea,
3. 3-(3-fluoro-4-(1H-imidazol-1-yl)pyridin-2-yl)-1-(2-methoxypyrimidin-5-yl)-1-((5-(trifluoromethyl)-1H-pyrazol-3-yl)methyl)urea,
4. 3-(2-bromo-3-fluoropyridin-4-yl)-1-(2-methoxypyrimidin-5-yl)-1-((5-(trifluoromethyl)-1H-pyrazol-3-yl)methyl)urea,
5. 3-(6-chloropyridin-2-yl)-1-(2-methoxypyrimidin-5-yl)-1-((5-(trifluoromethyl)-1H-pyrazol-3-yl)methyl)urea,
6. 3-(3-chlorobicyclo[1.1.1]pentan-1-yl)-1-(2-methoxypyrimidin-5-yl)-1-((5-(trifluoromethyl)-1H-pyrazol-3-yl)methyl)urea,
7. 3-(3-chloro-4-cyanophenyl)-1-(2-methoxypyrimidin-5-yl)-1-((5-(trifluoromethyl)-1H-pyrazol-3-yl)methyl)urea,
8. 3-(5-chloro-6-cyanopyridin-2-yl)-1-(2-methoxypyrimidin-5-yl)-1-((5-(trifluoromethyl)-1H-pyrazol-3-yl)methyl)urea,
9. 3-(3-chloro-4-isocyanophenyl)-1-((5-cyano-1H-pyrazol-3-yl)methyl)-1-(2-methoxypyrimidin-5-yl)urea,
10. 3-(3-cyano-4,5-difluorophenyl)-1-((5-(difluoromethyl)-1H-pyrazol-3-yl)methyl)-1-(2-methoxypyrimidin-5-yl)urea,
11. 3-(3-chloro-4,5-difluorophenyl)-1-(2-methoxypyrimidin-5-yl)-1-((5-(trifluoromethyl)-1H-pyrazol-3-yl)methyl)urea,
12. 3-(3-chloro-4,5-difluorophenyl)-1-((5-(difluoromethyl)-1H-pyrazol-3-yl)methyl)-1-(2-methoxypyrimidin-5-yl)urea,
13. 3-(6-chloro-4,5-difluoropyridin-2-yl)-1-(2-methoxypyrimidin-5-yl)-1-((5-(trifluoromethyl)-1H-pyrazol-3-yl)methyl)urea,
14. 3-(3-chloro-4-fluorophenyl)-1-(6-oxo-1,6-dihydropyridin-3-yl)-1-((4,5,6,7-tetrahydro-1H-indazol-3-yl)methyl)urea,
15. 3-(3-chloro-4-fluorophenyl)-1-(2-oxo-1,2-dihydropyridin-4-yl)-1-((4,5,6,7-tetrahydro-1H-indazol-3-yl)methyl)urea,
16. N-(4-(3-(3-chloro-4-fluorophenyl)-1-((1,4,5,7-tetrahydropyrano[3,4-c]pyrazol-3-yl)methyl)ureido)phenyl)methanesulfonamide,
17. N-(5-(3-(3-chloro-4-fluorophenyl)-1-((1,4,5,7-tetrahydropyrano[3,4-c]pyrazol-3-yl)methyl)ureido)pyridin-2-yl)methanesulfonamide,
18. N-(5-(3-(3-chloro-4-fluorophenyl)-1-((1,4,5,6-tetrahydropyrano[2,3-c]pyrazol-3-yl)methyl)ureido)pyridin-2-yl)methanesulfonamide,
19. N-(4-(3-(3-chloro-4-fluorophenyl)-1-((1,4,5,6-tetrahydropyrano[2,3-c]pyrazol-3-yl)methyl)ureido)phenyl)methanesulfonamide,
20. 1-(6-acetylpyridin-3-yl)-3-(3-chloro-4-fluorophenyl)-1-((1,4,5,6-tetrahydropyrano[2,3-c]pyrazol-3-yl)methyl)urea,
21. 1-(6-acetylpyridin-3-yl)-3-(3-chloro-4-fluorophenyl)-1-((1,4,5,7-tetrahydropyrano[3,4-c]pyrazol-3-yl)methyl)urea,
22. 1-(2-acetylpyridin-4-yl)-3-(3-chloro-4-fluorophenyl)-1-((1,4,5,7-tetrahydropyrano[3,4-c]pyrazol-3-yl)methyl)urea,
23. 4-(3-(3-chloro-4-fluorophenyl)-1-((1,4,5,7-tetrahydropyrano[3,4-c]pyrazol-3-yl)methyl)ureido)-N-methylbenzenesulfonamide,
24. 3-(3-chloro-4-fluorophenyl)-1-(1-methyl-1H-pyrazol-4-yl)-1-((4,5,6,7-tetrahydro-1H-indazol-3-yl)methyl)urea,
25. 3-(3-chloro-4-fluorophenyl)-1-(1-methyl-1H-pyrazol-3-yl)-1-((1,4,5,6-tetrahydropyrano[2,3-c]pyrazol-3-yl)methyl)urea,
26. 3-(3-chloro-4-fluorophenyl)-1-(5-methylisoxazol-3-yl)-1-((1,4,5,6-tetrahydropyrano[2,3-c]pyrazol-3-yl)methyl)urea,
27. 3-(3-chloro-4-fluorophenyl)-1-(3-methylbicyclo[1.1.1]pentan-1-yl)-1-((5-(trifluoromethyl)-1H-pyrazol-3-yl)methyl)urea,
28. 3-(3-chloro-4-fluorophenyl)-1-(2-methoxy-4-methylpyrimidin-5-yl)-1-((5-(trifluoromethyl)-1H-pyrazol-3-yl)methyl)urea,
29. 3-(3-chloro-4-fluorophenyl)-1-(4-methoxy-2,6-dimethylphenyl)-1-((5-(trifluoromethyl)-1H-pyrazol-3-yl)methyl)urea,
30. 3-(3-chloro-4-fluorophenyl)-1-(2-methoxy-4,6-dimethylpyrimidin-5-yl)-1-((5-(trifluoromethyl)-1H-pyrazol-3-yl)methyl)urea,
31. methyl 4-(3-(3-chloro-4-fluorophenyl)-1-((4,5,6,7-tetrahydro-1H-indazol-3-yl)methyl)ureido)benzoate,
32. 3-(3-chloro-4-fluorophenyl)-1-(4-methoxyphenyl)-1-((4,5,6,7-tetrahydro-1H-indazol-3-yl)methyl)urea,
33. 1-((5-acetyl-4,5,6,7-tetrahydro-1H-pyrazolo[4,3-c]pyridin-3-yl)methyl)-3-(3-chloro-4-fluorophenyl)-1-(4-methoxyphenyl)urea,
34. 1-((1H-pyrazol-3-yl)methyl)-3-(3-chloro-4-fluorophenyl)-1-(4-methoxyphenyl)urea,
35. 3-(3-chloro-4-fluorophenyl)-1-(4-methoxyphenyl)-1-((1-methyl-1H-pyrazol-3-yl)methyl)urea,
36. 3-(3-chloro-4-fluorophenyl)-1-(4-methoxyphenyl)-1-((1-methyl-4,5,6,7-tetrahydro-1H-indazol-3-yl)methyl)urea,
37. 3-(3-chloro-4-fluorophenyl)-1-(4-methoxyphenyl)-1-((5-methyl-4,5,6,7-tetrahydro-1H-pyrazolo[4,3-c]pyridin-3-yl)methyl)urea,
38. 3-(3-chloro-4-fluorophenyl)-1-(4-methoxyphenyl)-1-((1,4,6,7-tetrahydropyrano[4,3-c]pyrazol-3-yl)methyl)urea,
39. 3-(3-chloro-4-fluorophenyl)-1-(4-methoxyphenyl)-1-((1-methyl-1,4,6,7-tetrahydropyrano[4,3-c]pyrazol-3-yl)methyl)urea,
40. 3-(3-chloro-4-fluorophenyl)-1-((5,5-difluoro-4,5,6,7-tetrahydro-1H-indazol-3-yl)methyl)-1-(6-methoxypyridin-3-yl)urea,
41. 3-(3-chloro-4-fluorophenyl)-1-(6-methoxypyridin-3-yl)-1-((1,4,5,6-tetrahydrocyclopenta[c]pyrazol-3-yl)methyl)urea,
42. 3-(3-chloro-4-fluorophenyl)-1-(6-methoxypyridin-3-yl)-1-((4,5,6,7-tetrahydro-1H-indazol-3-yl)methyl)urea,
43. 1-((1H-indazol-3-yl)methyl)-3-(3-chloro-4-fluorophenyl)-1-(6-methoxypyridin-3-yl)urea,
44. 3-(3-chloro-4-fluorophenyl)-1-((5-(hydroxymethyl)-4,5,6,7-tetrahydro-1H-indazol-3-yl)methyl)-1-(6-methoxypyridin-3-yl)urea,
45. 3-(3-chloro-4-fluorophenyl)-1-(6-methoxypyridin-3-yl)-1-((1,4,6,7-tetrahydrothiopyrano[4,3-c]pyrazol-3-yl)methyl)urea,
46. 3-(3-chloro-4-fluorophenyl)-1-(4-methoxyphenyl)-1-((1,4,5,6-tetrahydrocyclopenta[c]pyrazol-3-yl)methyl)urea,
47. 3-(3-chloro-4-fluorophenyl)-1-((5,5-dimethyl-4,5,6,7-tetrahydro-1H-indazol-3-yl)methyl)-1-(6-methoxypyridin-3-yl)urea,
48. 3-(3-chloro-4-fluorophenyl)-1-((5-hydroxy-4,5,6,7-tetrahydro-1H-indazol-3-yl)methyl)-1-(6-methoxypyridin-3-yl)urea,
49. 3-(3-chloro-4-fluorophenyl)-1-((5,5-dioxido-1,4,6,7-tetrahydrothiopyrano[4,3-c]pyrazol-3-yl)methyl)-1-(6-methoxypyridin-3-yl)urea,
50. 3-(3-chloro-4-fluorophenyl)-1-(6-methoxypyridin-3-yl)-1-((5-methyl-1H-pyrazol-3-yl)methyl)urea,
51. 3-(3-chloro-4-fluorophenyl)-1-(6-methoxypyridin-3-yl)-1-((4,5,6,7-tetrahydro-1H-4,7-methanoindazol-3-yl)methyl)urea,
52. 3-((3-(3-chloro-4-fluorophenyl)-1-(6-methoxypyridin-3-yl)ureido)methyl)-4,5,6,7-tetra hydro-1H-indazole-5-carboxylic acid,
53. methyl 3-((3-(3-chloro-4-fluorophenyl)-1-(6-methoxypyridin-3-yl)ureido)methyl)-4,5,6,7-tetra hydro-1H-indazole-5-carboxylate,
54. 3-(3-chloro-4-fluorophenyl)-1-(2-methoxypyridin-4-yl)-1-((4,5,6,7-tetrahydro-1H-indazol-3-yl)methyl)urea,
55. 3-(3-chloro-4-fluorophenyl)-1-(6-methoxypyridin-3-yl)-1-((1,4,6,7-tetrahydropyrano[4,3-c]pyrazol-3-yl)methyl)urea,
56. 3-(3-chloro-4-fluorophenyl)-1-(6-methoxypyridin-3-yl)-1-((4-methyl-1H-pyrazol-3-yl)methyl)urea,
57. 3-(3-chloro-4-fluorophenyl)-1-((4,5-dimethyl-1H-pyrazol-3-yl)methyl)-1-(6-methoxypyridin-3-yl)urea,
58. 3-(3-chloro-4-fluorophenyl)-1-((5-isopropyl-1H-pyrazol-3-yl)methyl)-1-(6-methoxypyridin-3-yl)urea,
59. 3-(3-chloro-4-fluorophenyl)-1-(6-methoxypyridin-3-yl)-1-((6-methyl-1,4,5,6-tetrahydrocyclopenta[c]pyrazol-3-yl)methyl)urea,
60. 3-(3-chloro-4-fluorophenyl)-1-((5-ethyl-1H-pyrazol-3-yl)methyl)-1-(6-methoxypyridin-3-yl)urea,
61. 1-((4-bromo-1H-pyrazol-3-yl)methyl)-3-(3-chloro-4-fluorophenyl)-1-(4-methoxyphenyl)urea,
62. 3-(3-chloro-4-fluorophenyl)-1-(6-methoxypyridin-3-yl)-1-((5-(trifluoromethyl)-4,5,6,7-tetrahydro-1H-indazol-3-yl)methyl)urea,
63. 3-(3-chloro-4-fluorophenyl)-1-(6-methoxypyridin-3-yl)-1-((1,4,5,6-tetrahydropyrano[2,3-c]pyrazol-3-yl)methyl)urea,
64. 3-(3-chloro-4-fluorophenyl)-1-((5-methoxy-1H-pyrazol-3-yl)methyl)-1-(6-methoxypyridin-3-yl)urea,
65. 3-(3-chloro-4-fluorophenyl)-1-(1-methyl-6-oxo-1,6-dihydropyridin-3-yl)-1-((1,4,5,6-tetrahydrocyclopenta[c]pyrazol-3-yl)methyl)urea,
66. 3-(3-chloro-4-fluorophenyl)-1-(6-methoxypyridin-3-yl)-1-((1-(2-(tetrahydro-2H-pyran-2-yloxy)ethyl)-1,4,5,6-tetrahydrocyclopenta[c]pyrazol-3-yl)methyl)urea,
67. 3-(3-chloro-4-fluorophenyl)-1-((1-(2-hydroxyethyl)-1,4,5,6-tetrahydrocyclopenta[c]pyrazol-3-yl)methyl)-1-(6-methoxypyridin-3-yl)urea,
68. 3-(3-chloro-4-fluorophenyl)-1-((1,3a,4,5,6,7a-hexahydropyrano[2,3-c]pyrazol-3-yl)methyl)-1-(2-methoxypyridin-4-yl)urea,
69. 3-(3-chloro-4-fluorophenyl)-1-((4,6-dihydro-1H-furo[3,4-c]pyrazol-3-yl)methyl)-1-(6-methoxypyridin-3-yl)urea,
70. 3-(3-chloro-4-fluorophenyl)-1-((4,6-dihydro-1H-furo[3,4-c]pyrazol-3-yl)methyl)-1-(2-methoxypyridin-4-yl)urea,
71. 3-(3-chloro-4-fluorophenyl)-1-((5,6-dihydro-1H-[1,4]dioxino[2,3-c]pyrazol-3-yl)methyl)-1-(2-methoxypyridin-4-yl)urea,
72. 3-(3-chloro-4-fluorophenyl)-1-((5-(difluoromethyl)-1H-pyrazol-3-yl)methyl)-1-(6-methoxypyridin-3-yl)urea,
73. 3-(3-chloro-4-fluorophenyl)-1-(2-methoxypyridin-4-yl)-1-((1,4,6,7-tetrahydropyrano[4,3-c]pyrazol-3-yl)methyl)urea,
74. 3-(3-chloro-4-fluorophenyl)-1-(2-methoxypyridin-4-yl)-1-((5-(trifluoromethyl)-1H-pyrazol-3-yl)methyl)urea,
75. 3-(3-chloro-4-fluorophenyl)-1-(5-methoxypyridin-3-yl)-1-((1,4,6,7-tetrahydropyrano[4,3-c]pyrazol-3-yl)methyl)urea,
76. 3-(3-chloro-4-fluorophenyl)-1-(5-methoxypyridin-3-yl)-1-((1,4,5,6-tetrahydropyrano[2,3-c]pyrazol-3-yl)methyl)urea,
77. 3-(3-chloro-4-fluorophenyl)-1-((5-(difluoromethyl)-1H-pyrazol-3-yl)methyl)-1-(5-methoxypyridin-3-yl)urea,
78. 3-(3-chloro-4-fluorophenyl)-1-(6-methoxypyridin-3-yl)-1-((4-methyl-5-(trifluoromethyl)-1H-pyrazol-3-yl)methyl)urea,
79. 3-(3-chloro-4-fluorophenyl)-1-((1-(hydroxymethyl)-5-(trifluoromethyl)-1H-pyrazol-3-yl)methyl)-1-(6-methoxypyridin-3-yl)urea,
80. 3-(3-chloro-4-fluorophenyl)-1-(2-methoxypyrimidin-5-yl)-1-((5-(trifluoromethyl)-1H-pyrazol-3-yl)methyl)urea,
81. 1-((4-bromo-5-(difluoromethyl)-1H-pyrazol-3-yl)methyl)-3-(3-chloro-4-fluorophenyl)-1-(2-methoxypyrimidin-5-yl)urea,
82. 3-(3-chloro-4-fluorophenyl)-1-((5-(difluoromethyl)-4-(methoxymethyl)-1H-pyrazol-3-yl)methyl)-1-(6-methoxypyridin-3-yl)urea,
83. 3-(3-chloro-4-fluorophenyl)-1-((4-(2-ethoxyvinyl)-5-(trifluoromethyl)-1H-pyrazol-3-yl)methyl)-1-(6-methoxypyridin-3-yl)urea,
84. 3-(3-chloro-4-fluorophenyl)-1-((5-(difluoromethyl)-1H-pyrazol-3-yl)methyl)-1-(2-methoxypyrimidin-5-yl)urea,
85. 3-(3-chloro-4-fluorophenyl)-1-((4-(2-ethoxyethyl)-5-(trifluoromethyl)-1H-pyrazol-3-yl)methyl)-1-(6-methoxypyridin-3-yl)urea,
86. 3-(3-chloro-4-fluorophenyl)-1-((4-(2-hydroxyethyl)-5-(trifluoromethyl)-1H-pyrazol-3-yl)methyl)-1-(6-methoxypyridin-3-yl)urea,
87. 3-(3-chloro-4-fluorophenyl)-1-(6-methoxypyridin-3-yl)-1-((4-((4,4,5,5-tetramethyl-1,3-dioxolan-2-yl)methyl)-5-(trifluoromethyl)-1H-pyrazol-3-yl)methyl)urea,
88. 3-(3-chloro-4-fluorophenyl)-1-((5-(difluoromethyl)-4-(2-methoxyethyl)-1H-pyrazol-3-yl)methyl)-1-(6-methoxypyridin-3-yl)urea,
89. 3-(4-chloro-5-fluoropyridin-2-yl)-1-(2-methoxypyrimidin-5-yl)-1-((5-(trifluoromethyl)-1H-pyrazol-3-yl)methyl)urea,
90. 3-(3-chloro-4-fluorophenyl)-1-((5-methoxy-4-(trifluoromethyl)-1H-pyrazol-3-yl)methyl)-1-(6-methoxypyridin-3-yl)urea,
91. 3-(3-chloro-4-fluorophenyl)-1-((4-(2-hydroxyethyl)-5-(trifluoromethyl)-1H-pyrazol-3-yl)methyl)-1-(2-methoxypyrimidin-5-yl)urea,
92. 3-(3-chloro-4-fluorophenyl)-1-((5-(difluoromethyl)-4-(2-hydroxypropan-2-yl)-1H-pyrazol-3-yl)methyl)-1-(2-methoxypyrimidin-5-yl)urea,
93. 3-(3-chloro-4-fluorophenyl)-1-((5-(difluoromethyl)-4-(prop-1-en-2-yl)-1H-pyrazol-3-yl)methyl)-1-(2-methoxypyrimidin-5-yl)urea,
94. 3-(3-chloro-4-fluorophenyl)-1-(6-methoxypyridazin-4-yl)-1-((5-(trifluoromethyl)-1H-pyrazol-3-yl)methyl)urea,
95. 3-(3-chloro-4-fluorophenyl)-1-((4-(2-hydroxypropyl)-5-(trifluoromethyl)-1H-pyrazol-3-yl)methyl)-1-(2-methoxypyrimidin-5-yl)urea,
96. 3-(3-chloro-4-fluorophenyl)-1-(6-methoxypyridazin-3-yl)-1-((5-(trifluoromethyl)-1H-pyrazol-3-yl)methyl)urea,
97. 3-(3-chloro-4-fluorophenyl)-1-((5-(difluoromethyl)-1H-pyrazol-3-yl)methyl)-1-(6-methoxypyridazin-3-yl)urea,
98. 1-((4-bromo-5-(difluoromethyl)-1H-pyrazol-3-yl)methyl)-3-(3-chloro-4-fluorophenyl)-1-(2-methoxypyrimidin-5-yl)urea,
99. 3-(3-chloro-4-fluorophenyl)-1-((5-(difluoromethyl)-1H-pyrazol-3-yl)methyl)-1-(6-methoxypyridazin-4-yl)urea,
100. 3-(3-chloro-4-fluorophenyl)-1-((5-(difluoromethyl)-4-methoxy-1H-pyrazol-3-yl)methyl)-1-(6-methoxypyridin-3-yl)urea,
101. 3-(3-chloro-4-fluorophenyl)-1-((4-(2-hydroxy-2-methylpropyl)-5-(trifluoromethyl)-1H-pyrazol-3-yl)methyl)-1-(2-methoxypyrimidin-5-yl)urea,
102. 3-(3-chloro-4-fluorophenyl)-1-((4-(2-hydroxypropan-2-yl)-5-(trifluoromethyl)-1H-pyrazol-3-yl)methyl)-1-(2-methoxypyrimidin-5-yl)urea,
103. 3-(3-chloro-4-fluorophenyl)-1-((5-(difluoromethyl)-4-(2-hydroxy-2-methylpropyl)-1H-pyrazol-3-yl)methyl)-1-(2-methoxypyrimidin-5-yl)urea,
104. 3-(3-chloro-4-fluorophenyl)-1-((5-cyano-1H-pyrazol-3-yl)methyl)-1-(2-methoxypyrimidin-5-yl)urea,
105. 3-(3-chloro-4-fluorophenyl)-1-(3-methoxybicyclo[1.1.1]pentan-1-yl)-1-((5-(trifluoromethyl)-1H-pyrazol-3-yl)methyl)urea,
106. 3-(6-chloro-5-fluoropyridin-2-yl)-1-(2-methoxypyrimidin-5-yl)-1-((5-(trifluoromethyl)-1H-pyrazol-3-yl)methyl)urea,
107. 3-(5-chloro-6-fluoropyridin-3-yl)-1-(2-methoxypyrimidin-5-yl)-1-((5-(trifluoromethyl)-1H-pyrazol-3-yl)methyl)urea,
108. 3-(3-chloro-4-fluorophenyl)-1-((1,3-dimethyl-1H-pyrazol-5-yl)methyl)-1-(4-methoxyphenyl)urea,
109. 3-(3-chloro-4-fluorophenyl)-1-(3-(2-hydroxypropan-2-yl)isoxazol-5-yl)-1-((1,4,5,6-tetrahydropyrano[2,3-c]pyrazol-3-yl)methyl)urea,
110. 3-(3-chloro-4-fluorophenyl)-1-(6-(2-hydroxypropan-2-yl)pyridin-3-yl)-1-((1,4,5,6-tetrahydropyrano[2,3-c]pyrazol-3-yl)methyl)urea,
111. 3-(3-chloro-4-fluorophenyl)-1-(6-(2-hydroxypropan-2-yl)pyridin-3-yl)-1-((1,4,5,7-tetrahydropyrano[3,4-c]pyrazol-3-yl)methyl)urea,
112. 3-(3-chloro-4-fluorophenyl)-1-(2-(2-hydroxypropan-2-yl)pyridin-4-yl)-1-((1,4,5,7-tetrahydropyrano[3,4-c]pyrazol-3-yl)methyl)urea,
113. 3-(3-chloro-4-fluorophenyl)-1-(2-(2-hydroxypropan-2-yl)pyridin-4-yl)-1-((1,4,5,6-tetrahydropyrano[2,3-c]pyrazol-3-yl)methyl)urea,
114. 3-(3-chloro-4-fluorophenyl)-1-(4-hydroxyphenyl)-1-((1,4,5,6-tetrahydrocyclopenta[c]pyrazol-3-yl)methyl)urea,
115. 3-(3-chloro-4-fluorophenyl)-1-(3-(2-hydroxypropan-2-yl)isoxazol-5-yl)-1-((1,4,5,7-tetrahydropyrano[3,4-c]pyrazol-3-yl)methyl)urea,
116. 3-(3-chloro-4-fluorophenyl)-1-(3,4-dimethoxyphenyl)-1-((4,5,6,7-tetrahydro-1H-indazol-3-yl)methyl)urea,
117. 3-(3-chloro-4-fluorophenyl)-1-(3,5-dimethoxyphenyl)-1-((4,5,6,7-tetrahydro-1H-indazol-3-yl)methyl)urea,
118. 3-(3-chloro-4-fluorophenyl)-1-(6-cyano-5-methoxypyridin-3-yl)-1-((5-(trifluoromethyl)-1H-pyrazol-3-yl)methyl)urea,
119. 3-(3-chloro-4-fluorophenyl)-1-(5-cyano-6-methoxypyridin-3-yl)-1-((5-(trifluoromethyl)-1H-pyrazol-3-yl)methyl)urea,
120. 3-(3-chloro-4-fluorophenyl)-1-(5-cyanopyridin-3-yl)-1-((1,4,5,6-tetrahydropyrano[2,3-c]pyrazol-3-yl)methyl)urea,
121. 3-(3-chloro-4-fluorophenyl)-1-(6-cyanopyridin-3-yl)-1-((1,4,6,7-tetrahydropyrano[4,3-c]pyrazol-3-yl)methyl)urea,
122. 3-(3-chloro-4-fluorophenyl)-1-(2-cyanopyridin-4-yl)-1-((1,4,5,6-tetrahydropyrano[2,3-c]pyrazol-3-yl)methyl)urea,
123. 3-(3-chloro-4-fluorophenyl)-1-(2-cyanopyridin-4-yl)-1-((1,4,6,7-tetrahydropyrano[4,3-c]pyrazol-3-yl)methyl)urea,
124. 3-(3-chloro-4-fluorophenyl)-1-(6-cyanopyridin-3-yl)-1-((1,4,5,6-tetrahydropyrano[2,3-c]pyrazol-3-yl)methyl)urea,
125. 3-(3-chloro-4-fluorophenyl)-1-(6-cyanopyridin-3-yl)-1-((1,4,6,7-tetrahydropyrano[4,3-c]pyrazol-3-yl)methyl)urea,
126. 3-(3-chloro-4-fluorophenyl)-1-(6-cyanopyridin-3-yl)-1-((5-(difluoromethyl)-1H-pyrazol-3-yl)methyl)urea,
127. 3-(3-chloro-4-fluorophenyl)-1-(6-cyanopyridin-3-yl)-1-((1,4,5,7-tetrahydropyrano[3,4-c]pyrazol-3-yl)methyl)urea,
128. 3-(3-chloro-4-fluorophenyl)-1-(2-cyanopyridin-4-yl)-1-((1,4,5,7-tetrahydropyrano[3,4-c]pyrazol-3-yl)methyl)urea,
129. 3-(3-chloro-4-fluorophenyl)-1-(5-cyanopyridin-3-yl)-1-((1,4,6,7-tetrahydropyrano[4,3-c]pyrazol-3-yl)methyl)urea,
130. 3-(3-chloro-4-fluorophenyl)-1-(6-cyanopyridin-3-yl)-1-((5-(trifluoromethyl)-1H-pyrazol-3-yl)methyl)urea,
131. 4-(3-(3-chloro-4-fluorophenyl)-1-((4,5,6,7-tetrahydro-1H-indazol-3-yl)methyl)ureido)benzoic acid,
132. 3-(3-chloro-4-fluorophenyl)-1-(isoxazol-3-yl)-1-((4,5,6,7-tetrahydro-1H-indazol-3-yl)methyl)urea,
133. 3-(3-chloro-4-fluorophenyl)-1-((4,5,6,7-tetrahydro-1H-indazol-3-yl)methyl)-1-(1,3,4-thiadiazol-2-yl)urea,
134. 3-(3-chloro-4-fluorophenyl)-1-(isoxazol-3-yl)-1-((1,4,5,6-tetrahydrocyclopenta[c]pyrazol-3-yl)methyl)urea,
135. 3-(3-chloro-4-fluorophenyl)-1-(1H-pyrazol-3-yl)-1-((1,4,5,6-tetrahydropyrano[2,3-c]pyrazol-3-yl)methyl)urea,
136. 3-(3-chloro-4-fluorophenyl)-1-(isoxazol-3-yl)-1-((1,4,5,6-tetrahydropyrano[2,3-c]pyrazol-3-yl)methyl)urea,
137. 1-(bicyclo[1.1.1]pentan-1-yl)-3-(3-chloro-4-fluorophenyl)-1-((5-(trifluoromethyl)-1H-pyrazol-3-yl)methyl)urea,
138. 3-(6-chloro-4-(dimethylamino)-5-fluoropyridin-2-yl)-1-(2-methoxypyrimidin-5-yl)-1-((5-(trifluoromethyl)-1H-pyrazol-3-yl)methyl)urea,
139. 3-(3-chloro-4-fluoro-5-methylphenyl)-1-((5-(difluoromethyl)-1H-pyrazol-3-yl)methyl)-1-(2-methoxypyrimidin-5-yl)urea,
140. 3-(3-chloro-4-fluoro-5-methylphenyl)-1-(2-methoxypyrimidin-5-yl)-1-((5-(trifluoromethyl)-1H-pyrazol-3-yl)methyl)urea,
141. 3-(6-chloro-3-fluoro-4-methylpyridin-2-yl)-1-(2-methoxypyrimidin-5-yl)-1-((5-(trifluoromethyl)-1H-pyrazol-3-yl)methyl)urea,
142. 3-(6-chloro-5-fluoro-4-methylpyridin-2-yl)-1-(2-methoxypyrimidin-5-yl)-1-((5-(trifluoromethyl)-1H-pyrazol-3-yl)methyl)urea,
143. 3-(2-chloropyrimidin-5-yl)-1-(2-methoxypyrimidin-5-yl)-1-((5-(trifluoromethyl)-1H-pyrazol-3-yl)methyl)urea,
144. 3-(3-cyanophenyl)-1-(2-methoxypyrimidin-5-yl)-1-((5-(trifluoromethyl)-1H-pyrazol-3-yl)methyl)urea,
145. 3-(6-cyanopyridin-2-yl)-1-(2-methoxypyrimidin-5-yl)-1-((5-(trifluoromethyl)-1H-pyrazol-3-yl)methyl)urea,
146. 3-(2-cyanopyridin-4-yl)-1-(2-methoxypyrimidin-5-yl)-1-((5-(trifluoromethyl)-1H-pyrazol-3-yl)methyl)urea,
147. 3-(3-cyanophenyl)-1-((5-(difluoromethyl)-1H-pyrazol-3-yl)methyl)-1-(2-methoxypyrimidin-5-yl)urea,
148. 3-(6-cyanopyridin-2-yl)-1-(2-methoxypyrimidin-5-yl)-1-((5-(trifluoromethyl)-1H-pyrazol-3-yl)methyl)urea,
149. 3-(3-cyano-4,5-difluorophenyl)-1-(2-methoxypyrimidin-5-yl)-1-((5-(trifluoromethyl)-1H-pyrazol-3-yl)methyl)urea,
150. 3-(3-cyano-4,5-difluorophenyl)-1-((5-(difluoromethyl)-1H-pyrazol-3-yl)methyl)-1-(2-methoxypyrimidin-5-yl)urea,
151. 3-(6-cyano-4,5-difluoropyridin-2-yl)-1-(2-methoxypyrimidin-5-yl)-1-((5-(trifluoromethyl)-1H-pyrazol-3-yl)methyl)urea,
152. 3-(4-cyano-3-(difluoromethyl)phenyl)-1-(2-methoxypyrimidin-5-yl)-1-((5-(trifluoromethyl)-1H-pyrazol-3-yl)methyl)urea,
153. 1-((5-cyano-1H-pyrazol-3-yl)methyl)-3-(4-cyano-3-(difluoromethyl)phenyl)-1-(2-methoxypyrimidin-5-yl)urea,
154. 3-(3-cyano-4-fluorophenyl)-1-(2-methoxypyrimidin-5-yl)-1-((5-(trifluoromethyl)-1H-pyrazol-3-yl)methyl)urea,
155. 3-(3-cyano-4-fluorophenyl)-1-((5-(difluoromethyl)-1H-pyrazol-3-yl)methyl)-1-(2-methoxypyrimidin-5-yl)urea,
156. 3-(4-cyano-3-fluorophenyl)-1-(2-methoxypyrimidin-5-yl)-1-((5-(trifluoromethyl)-1H-pyrazol-3-yl)methyl)urea,
157. 1-((5-cyano-1H-pyrazol-3-yl)methyl)-3-(3-cyano-4-fluorophenyl)-1-(2-methoxypyrimidin-5-yl)urea,
158. 3-(2-cyano-3-fluoropyridin-4-yl)-1-(2-methoxypyrimidin-5-yl)-1-((5-(trifluoromethyl)-1H-pyrazol-3-yl)methyl)urea,
159. 3-(3-chloro-4-fluorophenyl)-1-((1,3-dimethyl-1H-pyrazol-4-yl)methyl)-1-(4-methoxyphenyl)urea,
160. 3-(6-cyano-5-fluoropyridin-2-yl)-1-(2-methoxypyrimidin-5-yl)-1-((5-(trifluoromethyl)-1H-pyrazol-3-yl)methyl)urea,
161. 3-(4-cyano-5-fluoropyridin-2-yl)-1-(2-methoxypyrimidin-5-yl)-1-((5-(trifluoromethyl)-1H-pyrazol-3-yl)methyl)urea,
162. 3-(3-cyano-4-fluorophenyl)-1-(3-methoxybicyclo[1.1.1]pentan-1-yl)-1-((5-(trifluoromethyl)-1H-pyrazol-3-yl)methyl)urea,
163. 3-(3-cyano-4-fluorophenyl)-1-(6-methoxypyridazin-4-yl)-1-((5-(trifluoromethyl)-1H-pyrazol-3-yl)methyl)urea,
164. 3-(6-cyano-4-(dimethylamino)-5-fluoropyridin-2-yl)-1-(2-methoxypyrimidin-5-yl)-1-((5-(trifluoromethyl)-1H-pyrazol-3-yl)methyl)urea,
165. 3-(2,6-dichloropyridin-3-yl)-1-(2-methoxypyrimidin-5-yl)-1-((5-(trifluoromethyl)-1H-pyrazol-3-yl)methyl)urea,
166. 3-(2,6-dichloropyridin-3-yl)-1-((5-(difluoromethyl)-1H-pyrazol-3-yl)methyl)-1-(2-methoxypyrimidin-5-yl)urea,
167. 3-(3,4-dicyanophenyl)-1-(2-methoxypyrimidin-5-yl)-1-((5-(trifluoromethyl)-1H-pyrazol-3-yl)methyl)urea,
168. 3-(3,4-difluorophenyl)-1-(6-methoxypyridin-3-yl)-1-((5-(trifluoromethyl)-1H-pyrazol-3-yl)methyl)urea,
169. 3-(3,6-difluoropyridin-2-yl)-1-(2-methoxypyrimidin-5-yl)-1-((5-(trifluoromethyl)-1H-pyrazol-3-yl)methyl)urea,
170. 3-(5,6-difluoropyridin-2-yl)-1-(2-methoxypyrimidin-5-yl)-1-((5-(trifluoromethyl)-1H-pyrazol-3-yl)methyl)urea,
171. 3-(5,6-difluoropyridin-3-yl)-1-(2-methoxypyrimidin-5-yl)-1-((5-(trifluoromethyl)-1H-pyrazol-3-yl)methyl)urea,
172. 3-(4,5-difluoropyridin-2-yl)-1-(2-methoxypyrimidin-5-yl)-1-((5-(trifluoromethyl)-1H-pyrazol-3-yl)methyl)urea,
173. 3-(3,4-difluoropyridin-2-yl)-1-(2-methoxypyrimidin-5-yl)-1-((5-(trifluoromethyl)-1H-pyrazol-3-yl)methyl)urea,
174. 3-(5-(difluoromethyl)pyridin-3-yl)-1-(2-methoxypyrimidin-5-yl)-1-((5-(trifluoromethyl)-1H-pyrazol-3-yl)methyl)urea,
175. 3-(2-(difluoromethyl)pyridin-4-yl)-1-(2-methoxypyrimidin-5-yl)-1-((5-(trifluoromethyl)-1H-pyrazol-3-yl)methyl)urea,
176. 3-(3-(difluoromethyl)phenyl)-1-(2-methoxypyrimidin-5-yl)-1-((5-(trifluoromethyl)-1H-pyrazol-3-yl)methyl)urea,
177. 3-(6-(difluoromethyl)pyridin-2-yl)-1-(2-methoxypyrimidin-5-yl)-1-((5-(trifluoromethyl)-1H-pyrazol-3-yl)methyl)urea,
178. 1-((5-(difluoromethyl)-1H-pyrazol-3-yl)methyl)-3-(3-(difluoromethyl)phenyl)-1-(2-methoxypyrimidin-5-yl)urea,
179. 1-((5-(difluoromethyl)-1H-pyrazol-3-yl)methyl)-3-(6-(difluoromethyl)pyridin-2-yl)-1-(2-methoxypyrimidin-5-yl) urea,
180. 1-((5-cyano-1H-pyrazol-3-yl)methyl)-3-(3-(difluoromethyl)phenyl)-1-(2-methoxypyrimidin-5-yl)urea,
181. 3-(3-(difluoromethyl)phenyl)-1-(3-methoxybicyclo[1.1.1]pentan-1-yl)-1-((5-(trifluoro methyl)-1H-pyrazol-3-yl)methyl)urea,
182. 1-((4-(2-ethoxyethyl)-5-(trifluoromethyl)-1H-pyrazol-3-yl)methyl)-3-(4-fluorophenyl)-1-(6-methoxypyridin-3-yl)urea,
183. 3-(6-fluoropyridin-2-yl)-1-(2-methoxypyrimidin-5-yl)-1-((5-(trifluoromethyl)-1H-pyrazol-3-yl)methyl)urea,
184. 3-(6-fluoropyridin-3-yl)-1-(2-methoxypyrimidin-5-yl)-1-((5-(trifluoromethyl)-1H-pyrazol-3-yl)methyl)urea,
185. 3-(3-fluorobicyclo[1.1.1]pentan-1-yl)-1-(2-methoxypyrimidin-5-yl)-1-((5-(trifluoromethyl)-1H-pyrazol-3-yl)methyl)urea,
186. 3-(2-fluoropyrimidin-5-yl)-1-(2-methoxypyrimidin-5-yl)-1-((5-(trifluoromethyl)-1H-pyrazol-3-yl)methyl)urea,
187. 3-(4-fluoropyridin-2-yl)-1-(2-methoxypyrimidin-5-yl)-1-((5-(trifluoromethyl)-1H-pyrazol-3-yl)methyl)urea,
188. 3-(3-(difluoromethyl)-4-fluorophenyl)-1-(6-methoxypyridin-3-yl)-1-((5-(trifluoromethyl)-1H-pyrazol-3-yl)methyl)urea,
189. 3-(3-(difluoromethyl)-4-fluorophenyl)-1-(2-methoxypyrimidin-5-yl)-1-((5-(trifluoromethyl)-1H-pyrazol-3-yl)methyl)urea,
190. 1-((5-(difluoromethyl)-1H-pyrazol-3-yl)methyl)-3-(3-(difluoromethyl)-4-fluorophenyl)-1-(2-methoxypyrimidin-5-yl)urea,
191. 1-((5-(difluoromethyl)-1H-pyrazol-3-yl)methyl)-3-(3-(difluoromethyl)-4-fluorophenyl)-1-(6-methoxypyridin-3-yl)urea,
192. 3-(4-(difluoromethyl)-5-fluoropyridin-2-yl)-1-(2-methoxypyrimidin-5-yl)-1-((5-(trifluoromethyl)-1H-pyrazol-3-yl)methyl)urea,
193. 3-(3-(difluoromethyl)-4-fluorophenyl)-1-((4-(2-hydroxyethyl)-5-(trifluoromethyl)-1H-pyrazol-3-yl)methyl)-1-(2-methoxypyrimidin-5-yl)urea,
194. 1-((4-bromo-5-(trifluoromethyl)-1H-pyrazol-3-yl)methyl)-3-(3-(difluoromethyl)-4-fluorophenyl)-1-(2-methoxypyrimidin-5-yl)urea,
195. 1-((5-(difluoromethyl)-1H-pyrazol-3-yl)methyl)-3-(3-(difluoromethyl)-4-fluorophenyl)-1-(6-methoxypyridazin-3-yl)urea,
196. 1-((5-(difluoromethyl)-1H-pyrazol-3-yl)methyl)-3-(6-(difluoromethyl)-5-fluoropyridin-2-yl)-1-(2-methoxypyrimidin-5-yl)urea,
197. 1-((4-bromo-5-(difluoromethyl)-1H-pyrazol-3-yl)methyl)-3-(3-(difluoromethyl)-4-fluorophenyl)-1-(2-methoxypyrimidin-5-yl)urea,
198. 1-((5-(difluoromethyl)-4-(2-hydroxypropan-2-yl)-1H-pyrazol-3-yl)methyl)-3-(3-(difluoromethyl)-4-fluorophenyl)-1-(2-methoxypyrimidin-5-yl)urea,
199. 3-(3-(difluoromethyl)-4-fluorophenyl)-1-((4-(2-hydroxypropyl)-5-(trifluoromethyl)-1H-pyrazol-3-yl)methyl)-1-(2-methoxypyrimidin-5-yl)urea,
200. 3-(3-(difluoromethyl)-4-fluorophenyl)-1-(6-methoxypyridazin-3-yl)-1-((5-(trifluoromethyl)-1H-pyrazol-3-yl)methyl)urea,
201. 1-((5-(difluoromethyl)-1H-pyrazol-3-yl)methyl)-3-(3-(difluoromethyl)-4-fluorophenyl)-1-(6-methoxypyridazin-4-yl)urea,
202. 3-(6-(difluoromethyl)-5-fluoropyridin-2-yl)-1-(2-methoxypyrimidin-5-yl)-1-((5-(trifluoromethyl)-1H-pyrazol-3-yl)methyl)urea,
203. 3-(3-(difluoromethyl)-4-fluorophenyl)-1-(6-methoxypyridazin-4-yl)-1-((5-(trifluoromethyl)-1H-pyrazol-3-yl)methyl)urea,
204. 3-(6-(difluoromethyl)-5-fluoropyridin-2-yl)-1-(2-methoxypyrimidin-5-yl)-1-((5-(trifluoromethyl)-1H-pyrazol-3-yl)methyl)urea,
205. 3-(3-(difluoromethyl)-4-fluorophenyl)-1-((4-(2-hydroxy-2-methylpropyl)-5-(trifluoromethyl)-1H-pyrazol-3-yl)methyl)-1-(2-methoxypyrimidin-5-yl)urea,
206. 3-(4-(difluoromethyl)-3-fluorophenyl)-1-(2-methoxypyrimidin-5-yl)-1-((5-(trifluoromethyl)-1H-pyrazol-3-yl)methyl)urea,
207. 3-(3-(difluoromethyl)-4-fluorophenyl)-1-((4-(2-hydroxypropan-2-yl)-5-(trifluoromethyl)-1H-pyrazol-3-yl)methyl)-1-(2-methoxypyrimidin-5-yl)urea,
208. 1-((5-(difluoromethyl)-4-(2-hydroxy-2-methylpropyl)-1H-pyrazol-3-yl)methyl)-3-(3-(difluoromethyl)-4-fluorophenyl)-1-(2-methoxypyrimidin-5-yl)urea,
209. 1-((5-cyano-1H-pyrazol-3-yl)methyl)-3-(3-(difluoromethyl)-4-fluorophenyl)-1-(2-methoxypyrimidin-5-yl)urea,
210. 1-((5-(difluoromethyl)-1H-pyrazol-3-yl)methyl)-3-(6-(difluoromethyl)-5-fluoropyridin-2-yl)-1-(2-methoxypyrimidin-5-yl)urea,
211. 3-(2-(difluoromethyl)-3-fluoropyridin-4-yl)-1-(2-methoxypyrimidin-5-yl)-1-((5-(trifluoromethyl)-1H-pyrazol-3-yl)methyl)urea,
212. 1-((5-cyano-1H-pyrazol-3-yl)methyl)-3-(6-(difluoromethyl)-5-fluoropyridin-2-yl)-1-(2-methoxypyrimidin-5-yl)urea,
213. 3-(3-(difluoromethyl)-4-fluorophenyl)-1-(3-methoxybicyclo[1.1.1]pentan-1-yl)-1-((5-(t rifluoromethyl)-1H-pyrazol-3-yl)methyl)urea,
214. 3-(6-(difluoromethyl)-5-fluoropyridin-2-yl)-1-(3-methoxybicyclo[1.1.1]pentan-1-yl)-1-((5-(trifluoromethyl)-1H-pyrazol-3-yl)methyl)urea,
215. 1-((5-cyano-1H-pyrazol-3-yl)methyl)-3-(4-fluoro-3-(trifluoromethyl)phenyl)-1-(2-methoxypyrimidin-5-yl)urea,
216. 1,3-bis(2-methoxypyrimidin-5-yl)-1-((5-(trifluoromethyl)-1H-pyrazol-3-yl)methyl)urea,
217. 1-(2-methoxypyrimidin-5-yl)-3-(3-methylbicyclo[1.1.1]pentan-1-yl)-1-((5-(trifluoromethyl)-1H-pyrazol-3-yl)methyl)urea,
218. 1-(2-methoxypyrimidin-5-yl)-3-(2-(methylthio)pyrimidin-5-yl)-1-((5-(trifluoromethyl)-1H-pyrazol-3-yl)methyl)urea,
219. 1-(2-methoxypyrimidin-5-yl)-1-((5-(trifluoromethyl)-1H-pyrazol-3-yl)methyl)-3-(3,4,5-trifluorophenyl)urea,
220. 1-((5-(difluoromethyl)-1H-pyrazol-3-yl)methyl)-1-(2-methoxypyrimidin-5-yl)-3-(3,4,5-trifluorophenyl)urea,
221. 1-(2-methoxypyrimidin-5-yl)-1-((5-(trifluoromethyl)-1H-pyrazol-3-yl)methyl)-3-(3,5,6-trifluoropyridin-2-yl)urea,
222. 1-(3-methoxybicyclo[1.1.1]pentan-1-yl)-1-((5-(trifluoromethyl)-1H-pyrazol-3-yl)methyl)-3-(3,4,5-trifluorophenyl)urea,
223. 1-((5-cyano-1H-pyrazol-3-yl)methyl)-1-(2-methoxypyrimidin-5-yl)-3-(3,4,5-trifluorophenyl)urea,
224. 1-(2-methoxypyrimidin-5-yl)-1-((5-(trifluoromethyl)-1H-pyrazol-3-yl)methyl)-3-(4,5,6-trifluoropyridin-2-yl)urea,
225. 1-(2-methoxypyrimidin-5-yl)-1-((5-(trifluoromethyl)-1H-pyrazol-3-yl)methyl)-3-(6-(trifluoromethyl)pyridin-2-yl)urea, or
226. 3-(3-cyano-4-fluorophenyl)-1-(2-methoxypyrimidin-5-yl)-1-((1-propionyl-5-(trifluoro methyl)-1H-pyrazol-3-yl)methyl)urea, but is not limited thereto.

The compounds of the present invention may exist in the form of pharmaceutically acceptable salts. As the salts, acid addition salts formed by pharmaceutically acceptable free acids are useful. As used herein, the term “pharmaceutically acceptable salt” means all organic or inorganic addition salts of the compounds which have a concentration that is relatively non-toxic and harmless to patients and exhibits an effective action, and which cause side effects that do not diminish the beneficial efficacy of the compound represented by Chemical Formula 1.

An acid addition salt is prepared by way of a conventional method, for example, by dissolving the compound in an excess amount of an aqueous acid solution and precipitating this salt using a water-miscible organic solvent such as methanol, ethanol, acetone, or acetonitrile. Equimolar amounts of the compound and an acid or alcohol in water (for example, glycol monomethyl ether) may be heated, and then the mixture may be evaporated to dryness, or the precipitated salt may be subjected to suction filtration.

Here, organic acids and inorganic acids may be used as free acids. hydrochloric acid, phosphoric acid, sulfuric acid, nitric acid, tartaric acid and the like may be used as the inorganic acids. Methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like may be used as the organic acids. The free acids are not limited thereto.

The pharmaceutically acceptable metal salts may be prepared using bases. Alkali metal salts or alkaline earth metal salts are obtained by, for example, dissolving the compound in an excess amount of an alkali metal hydroxide or alkaline earth metal hydroxide solution, filtering the undissolved compound salt, and then evaporating and drying the filtrate. Here, it is pharmaceutically suitable to prepare a sodium, potassium, or calcium salt as the metal salts, but the metal salts are not limited thereto. A corresponding silver salt may be obtained by reacting an alkali metal or alkaline earth metal salt with a suitable silver salt (for example, silver nitrate).

The pharmaceutically acceptable salts of the compounds of the present invention include salts of acidic or basic groups that may be present in the compound represented by Chemical Formula 1 unless otherwise indicated. For example, the pharmaceutically acceptable salts may include sodium, calcium, and potassium salts of a hydroxyl group, and the like, other pharmaceutically acceptable salts of amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts, and these salts may be prepared by way of methods for preparing salts known in the art.

As the salts of the pyrazolylmethylurea derivative compounds of the present invention, all salts of pyrazolylmethylurea derivative compounds that are pharmaceutically acceptable salts and exhibit pharmacological activity equivalent to that of the pyrazolylmethylurea derivative compounds may be used without limitation.

The compound represented by Chemical Formula 1 according to the present invention includes not only pharmaceutically acceptable salts thereof but also solvates such as hydrates that can be prepared from the salts and all possible stereoisomers without limitation. The solvates and stereoisomers of the compound represented by Chemical Formula 1 may be prepared from the compound represented by Chemical Formula 1 by methods known in the art.

Furthermore, the compound represented by Chemical Formula 1 according to the present invention may be prepared in a crystalline form or an amorphous form, and may optionally be hydrated or solvated when prepared in a crystalline form. In the present invention, compounds containing various amounts of water as well as stoichiometric hydrates of the compound represented by Chemical Formula 1 may be included. The solvates of the compound represented by Chemical Formula 1 according to the present invention include both stoichiometric solvates and non-stoichiometric solvates.

A second aspect of the present invention provides a method for preparing the compound of the first aspect or a pharmaceutically acceptable salt thereof, which includes a first step of reacting a pyrazolylmethyl precursor with an R₁ precursor; and a second step of reacting the compound obtained in the previous step with an R₂ precursor.

For example, the reaction in the first step may be carried out by way of any one of the following Reaction Schemes 1 to 3, but is not limited thereto:

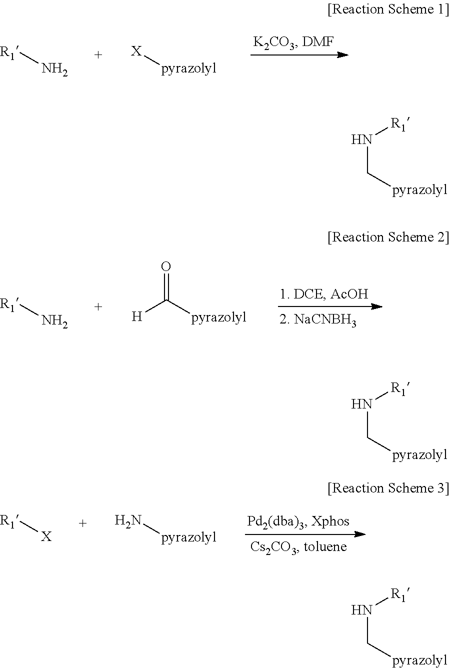

For example, the reaction in the second step may be carried out by way of any one of the following Reaction Schemes 4 to 7, but is not limited thereto:

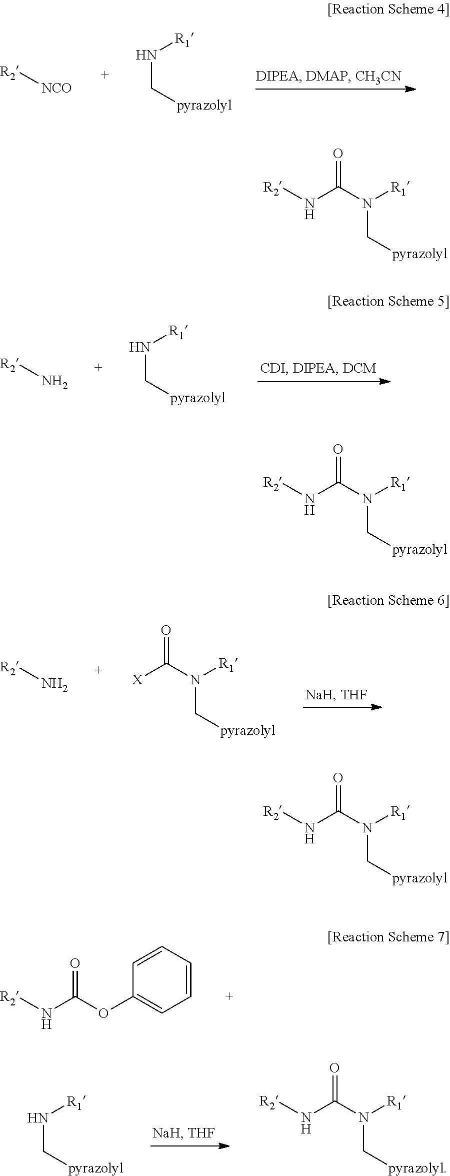

In the series of reaction schemes, pyrazolyl is an unsubstituted or substituted pyrazole ring, the nitrogen atom thereof may include a protecting group, R₁′ and R₂′ are the same as R₁′ and R₂′ or precursors thereof, respectively, and X is a halogen or another leaving group.

A third aspect of the present invention provides a composition for capsid assembly inhibition, containing the compound of the first aspect or a pharmaceutically acceptable salt thereof.

As used herein, the terms “compound of the first aspect” and “pharmaceutically acceptable salt” are as described above.

As used herein, the term “capsid” refers to a protein construct of a virus surrounding the genetic materials and enzymes required for reverse transcription, and is composed of subunits of several oligomeric (repetitive) structures of proteins called protomers. Observable three-dimensional morphological subunits that may or may not correspond to individual proteins are called capsomeres. The proteins that constitute the capsid are called capsid proteins or viral coat proteins (VCP). The capsid and its genome are called nucleocapsids. The capsids are broadly classified depending on their structures, and most viruses have capsids of a helical or icosahedral structure. Some viruses, such as bacteriophages, have developed into more complex structures because of the limitations in elasticity and electrostatics. The capsid surface may be composed of one or more proteins; for example, a foot-and-mouth disease virus capsid has a surface composed of the three proteins VP1-3. When a virus infects a cell and begins replicating itself, a new capsid subunit is synthesized using the protein biosynthesis mechanism of the cell. The genetic material encapsulated by the capsid may be RNA or DNA, but is not limited thereto.

For example, when infected with a virus, the host cell must rapidly produce thousands of identical copies of the original virus. When not inside an infected cell or in the process of infecting a cell, the virus exists in the form of (i) a genetic material, namely, long molecules of DNA or RNA that encode the protein needed by the virus to proliferate itself; (ii) capsids that are protein coats to surround and protect the genetic material; and optionally (iii) independent particles or virions that are surrounded by a lipid envelope and define viral identity.

A fourth aspect of the present invention provides an antiviral composition containing the compound of the first aspect or a pharmaceutically acceptable salt thereof as an active ingredient.

As used herein, the terms “compound of the first aspect” and “pharmaceutically acceptable salt” are as described above.

A fifth aspect of the present invention provides a pharmaceutical composition for prevention or treatment of viral disease, containing the compound of the first aspect or a pharmaceutically acceptable salt thereof as an active ingredient.

As used herein, the terms “compound of the first aspect” and “pharmaceutically acceptable salt” are as described above.

As used herein, the term “prevention” means any action in which the occurrence, spread, and recurrence of a viral disease is suppressed or delayed by administration of the composition of the present invention, and the term “treatment” means any action in which the symptoms of the disease are improved or advantageously changed by administration of the composition of the present invention.

The pharmaceutical composition of the present invention can prevent or treat diseases caused by viral infection by promoting the formation of an abnormal capsid in which the genetic material and elements replicating the genetic material are removed.

The viral disease may be an infectious disease caused by hepatitis B virus (HBV), hepatitis C virus (HCV), or human immunodeficiency virus (HIV), but is not limited thereto.

The pharmaceutical composition according to the present invention may contain the compound represented by Chemical Formula 1 or a pharmaceutically acceptable salt thereof as an active ingredient at preferably 0.1% to 75% by weight, more preferably 1% to 50% by weight based on the total weight of the composition.

The composition of the present invention may further contain a pharmaceutically acceptable carrier, diluent, or excipient, may be formulated and used in various forms such as oral formulations such as powders, granules, tablets, capsules, suspensions, emulsions, syrups, and aerosols and injections of sterile injection solutions by way of conventional methods depending on each purpose of use, and may be administered orally or through various routes including intravenous, intraperitoneal, subcutaneous, rectal, topical, and the like. Examples of the suitable carrier, excipient, or diluent that may be contained in such a composition include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, and mineral oil. The composition of the present invention may further contain a filler, an anti-agglomeration agent, a lubricant, a wetting agent, a flavoring agent, an emulsifier, a preservative, and the like.

Solid preparations for oral administration include tablets, pills, powders, granules, capsules, and the like, and such solid preparations are formulated by mixing at least one or more excipients such as starch, calcium carbonate, sucrose, lactose, or gelatin with the composition. In addition to simple excipients, lubricants such as magnesium stearate and talc may be used.

Liquid preparations for oral administration may include suspensions, internal solutions, emulsions, and syrups, and may contain various excipients such as wetting agents, sweetening agents, fragrances, and preservatives in addition to water and liquid paraffin that are commonly used simple diluents.

Preparations for parenteral administration include sterilized aqueous solutions, non-aqueous solutions, suspensions, emulsions, freeze-dried preparations, and suppositories. As non-aqueous solvents and suspending agents, propylene glycol, polyethylene glycol, vegetable oils such as olive oil, injectable esters such as ethyl oleate, and the like may be used. As the base of suppositories, Witepsol, Macrogol, Tween 61, cacao butter, laurin, glycerogelatin and the like may be used. Meanwhile, the injections may contain conventional additives such as solubilizers, isotonic agents, suspending agents, emulsifiers, stabilizers, and preservatives.

Here, the composition of the present invention is administered in a pharmaceutically effective amount. As used herein, the term “pharmaceutically effective amount” refers to an amount sufficient to treat a disease at a reasonable benefit/risk ratio applicable to medical treatment and not to cause side effects, and the effective dose level may be determined depending on factors including patient's health condition, the kind and severity of disease, drug activity, sensitivity to drug, method of administration, time of administration, route of administration and excretion rate, duration of treatment, mixing, or concomitant drugs, and other factors well known in the medical art. The composition of the present invention may be administered as an individual therapeutic agent or in combination with other therapeutic agents, may be administered sequentially or simultaneously with conventional therapeutic agents, and may be administered in a single or multiple manner. It is important to administer the composition in an amount so that the maximum effect can be acquired with a minimum amount without side effects in consideration of all the above factors, and the amount can be easily determined by those skilled in the art.

For example, the pharmaceutically effective amount may be increased or decreased depending on the route of administration, the severity of disease, gender, weight, age, and the like, and thus the dosage is not intended to limit the scope of the present invention in any way.

Specifically, the effective amount of the compound in the composition of the present invention may vary depending on the age, gender, and weight of the patient, and the compound may be administered generally at 1 mg to 100 mg, preferably 5 mg to 60 mg per kg of weight every day or every other day or 1 to 3 times a day in a divided manner. However, the effective amount may be increased or decreased depending on the route of administration, the severity of disease, gender, weight, age, and the like, and thus the dosage is not intended to limit the scope of the present invention in any way.

A sixth aspect of the present invention provides a method for treating a viral disease, which includes administering the pharmaceutical composition of the fifth aspect to an individual in need thereof.

As used herein, the terms “pharmaceutical composition of the fifth aspect” and “viral disease” are the same as described above.

As used herein, the term “individual” refers to all animals including monkeys, cows, horses, sheep, pigs, chickens, turkeys, quails, cats, dogs, mice, rats, rabbits, or guinea pigs, and humans, who have or may develop the viral diseases. By administering the pharmaceutical composition of the present invention to an individual, the disease can be effectively prevented or treated. The pharmaceutical composition of the present invention may be administered in combination with a conventional therapeutic agent.

As used herein, the term “administration” means to supply a predetermined substance to a patient by way of an arbitrary suitable method, and the composition of the present invention may be administered through any general route as long as it can reach the target tissue. The administration may be intraperitoneal administration, intravenous administration, intramuscular administration, subcutaneous administration, intradermal administration, oral administration, topical administration, intranasal administration, intrapulmonary administration, or rectal administration, but is not limited thereto. The pharmaceutical composition of the present invention may be administered using an arbitrary device capable of transporting an active substance to a target cell. Preferred administration modes and preparations are intravenous injections, subcutaneous injections, intradermal injections, intramuscular injections, drip injections, and the like. The injections may be prepared using aqueous solvents such as physiological saline solution and Ringer's solution, non-aqueous solvents such as vegetable oil, higher fatty acid esters (for example, ethyl oleate), alcohols (for example, ethanol, benzyl alcohol, propylene glycol, and glycerin), and the like. The injections may contain pharmaceutical carriers such as stabilizers to prevent deterioration (for example, ascorbic acid, sodium hydrogen sulfite, sodium pyrosulfite, BHA, tocopherol, and EDTA), emulsifiers, buffers for pH adjustment, and preservatives to inhibit the growth of microorganisms (for example, phenylmercuric nitrate, thimerosal, benzalkonium chloride, phenol, cresol, and benzyl alcohol).

As used herein, the term “therapeutically effective amount” used in combination with an active ingredient means an amount of a pyrazolylmethylurea derivative compound or a pharmaceutically acceptable salt thereof effective in the prevention or treatment of a target disease.

The pharmaceutical composition of the present invention may further contain a known drug used for the prevention or treatment of each known disease other than the pyrazolylmethylurea derivative compound or a pharmaceutically acceptable salt thereof as an active ingredient depending on the kind of disease to be prevented or treated. For example, when used for the prevention or treatment of viral diseases, the pharmaceutical composition of the present invention may further contain a known drug other than the pyrazolylmethylurea derivative compound or a pharmaceutically acceptable salt thereof as an active ingredient, and may be used in combination with other known treatments for the treatment of these diseases.

### MODE FOR THE INVENTION

Hereinafter, the configuration and effects of the present invention will be described in more detail with reference to exemplary embodiments. However, these exemplary embodiments are for illustrative purposes only, and the scope of the present invention is not intended to be limited by these exemplary embodiments.

### Synthesis Example

The compounds of the present invention can be synthesized through a process including a two-step reaction selected from the following series of reaction schemes.

Step 1: Reaction of Pyrazolylmethyl Precursor with R₁ Precursor

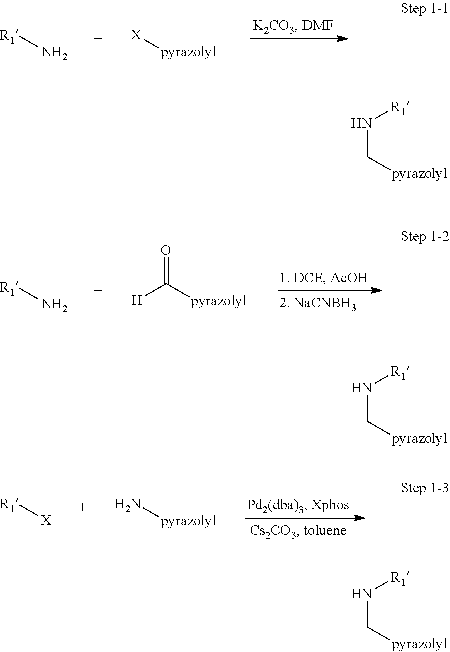

Step 2: Reaction with R₂ Precursor

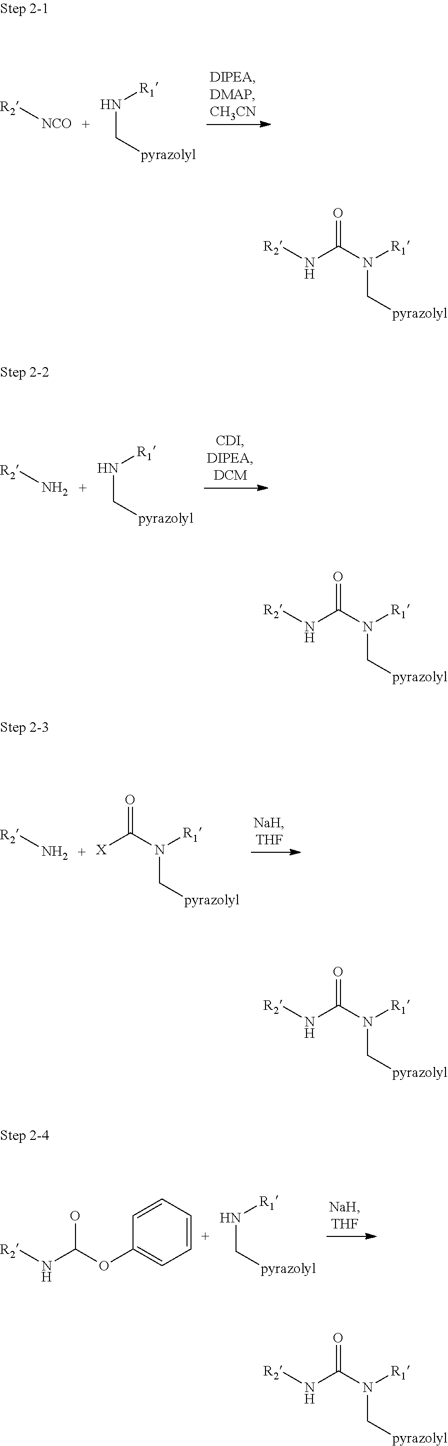

In the series of reaction schemes, pyrazolyl is an unsubstituted or substituted pyrazole ring, the nitrogen atom thereof may include a protecting group, R₁′ and R₂′ are the same as R₁′ and R₂′ or precursors thereof, respectively, and X is a halogen.

### Example 1. Preparation of 3-(bicyclo[1.1.1]pentan-1-yl)-1-(2-methoxypyrimidin-5-yl)-1-((5-(trifluoromethyl)-1H-pyrazol-3-yl)methyl)urea

[M+H⁺]=383.2,

¹H NMR (400 MHz, CDCl₃) δ 8.33-8.22 (m, 2H), 6.28 (s, 1H), 4.66 (s, 1H), 4.62 (s, 2H), 4.05 (s, 3H), 2.44 (s, 1H), 2.02 (s, 6H).

### Example 2. Preparation of 1-(2-methoxypyrimidin-5-yl)-3-(pyrimidin-5-yl)-1-((5-(trifluoromethyl)-1H-pyrazol-3-yl)methyl)urea

[M+H⁺]=395.2,

¹H NMR (400 MHz, DMSO-d₆) δ 8.85 (s, 2H), 8.80 (s, 1H), 8.58 (s, 2H), 6.65 (s, 1H), 4.92 (s, 2H), 3.95 (s, 3H).

### Example 3. Preparation of 3. 3-(3-fluoro-4-(1H-imidazol-1-yl)pyridin-2-yl)-1-(2-methoxypyrimidin-5-yl)-1-((5-(trifluoromethyl)-1H-pyrazol-3-yl)methyl)urea

[M+H]⁺=478.2,

¹H NMR (400 MHz, DMSO-d₆) δ 13.88-13.27 (m, 1H), 9.46-9.04 (m, 1H), 8.49 (s, 2H), 8.30-8.26 (m, 1H), 8.24 (s, 1H), 7.75-7.71 (m, 1H), 7.65-7.60 (m, 1H), 7.23-7.20 (m, 1H), 6.64-6.60 (m, 1H), 5.01-4.86 (m, 2H), 3.96-3.88 (m, 3H).

### Example 4. Preparation of 3-(2-bromo-3-fluoropyridin-4-yl)-1-(2-methoxypyrimidin-5-yl)-1-((5-(trifluoromethyl)-1H-pyrazol-3-yl)methyl)urea

[M+H⁺]=492.15,

¹H NMR (300 MHz, Chloroform-d) δ 11.26 (s, 1H), 8.43 (t, J=1.6 Hz, 2H), 8.17 (tt, J=9.5 Hz, 6.6 Hz, 2H), 6.58 (s, 1H), 6.41 (d, J=2.2 Hz, 1H), 4.81 (d, J=2.2 Hz, 2H), 4.13 (t, J=1.6 Hz, 3H).

### Example 5. Preparation of 3-(6-chloropyridin-2-yl)-1-(2-methoxypyrimidin-5-yl)-1-((5-(trifluoromethyl)-1H-pyrazol-3-yl)methyl)urea

[M+H⁺]=428. 1,

¹H NMR (400 MHz, CDCl₃) δ 11.19 (br s, 1H), 8.36 (s, 2H), 8.10-8.01 (m, 1H), 7.68 (t, J=8.0 Hz, 1H), 7.04 (d, J=7.6 Hz, 1H), 6.79 (s, 1H), 6.35 (s, 1H), 4.75 (s, 2H), 4.11 (s, 3H).

### Example 6. Preparation of 3-(3-chlorobicyclo[1.1.1]pentan-1-yl)-1-(2-methoxypyrimidin-5-yl)-1-((5-(trifluoromethyl)-1H-pyrazol-3-yl)methyl)urea

[M+H⁺]=417.2,

¹H NMR (400 MHz, CDCl₃) δ 11.69-11.18 (m, 1H), 8.27 (s, 2H), 6.30 (s, 1H), 4.78 (s, 1H), 4.64 (s, 2H), 4.06 (s, 3H), 2.39 (s, 6H).

### Example 7. Preparation of 3-(3-chloro-4-cyanophenyl)-1-(2-methoxypyrimidin-5-yl)-1-((5-(trifluoromethyl)-1H-pyrazol-3-yl)methyl)urea

¹H NMR (400 MHz, DMSO-d₆) δ 8.57 (s, 2H), 7.90 (d, J=2.0 Hz, 1H), 7.82 (d, J=8.7 Hz, 1H), 7.61 (dd, J=8.7 Hz, 2.0 Hz, 1H), 6.65 (s, 1H), 4.92 (s, 2H), 3.95 (s, 3H).

### Example 8. Preparation of 3-(5-chloro-6-cyanopyridin-2-yl)-1-(2-methoxypyrimidin-5-yl)-1-((5-(trifluoromethyl)-1H-pyrazol-3-yl)methyl)urea

¹H NMR (300 MHz, Chloroform-d) δ 11.14 (s, 1H), 8.39 (d, J=10.0 Hz, 3H), 7.87 (d, J=9.2 Hz, 1H), 6.94 (s, 1H), 6.40 (s, 1H), 4.79 (s, 2H), 4.14 (s, 3H).

### Example 9. Preparation of 3-(3-chloro-4-isocyanophenyl)-1-((5-cyano-1H-pyrazol-3-yl)methyl)-1-(2-methoxypyrimidin-5-yl)urea

[M+H⁺]=409.4,

¹H NMR (500 MHz, Methanol-d₄) δ 8.45 (s, 2H), 7.90 (s, 1H), 7.66 (d, J=8.7 Hz, 1H), 7.54 (d, J=9.8 Hz, 1H), 6.78 (s, 1H), 4.98 (s, 2H), 4.07 (s, 3H).

### Example 10. Preparation of 3-(3-cyano-4,5-difluorophenyl)-1-((5-(difluoromethyl)-1H-pyrazol-3-yl)methyl)-1-(2-methoxypyrimidin-5-yl)urea

[M+H⁺]=436.1,

¹H NMR (400 MHz, CDCl₃) δ=8.42 (s, 2H), 7.84 (ddd, J=1.9, 3.8, 9.3 Hz, 1H), 7.43 (q, J=9.4 Hz, 1H), 6.87-6.55 (m, 2H), 6.36 (s, 1H), 4.78 (s, 2H), 4.08 (s, 3H).

### Example 11. Preparation of 3-(3-chloro-4,5-difluorophenyl)-1-(2-methoxypyrimidin-5-yl)-1-((5-(trifluoromethyl)-1H-pyrazol-3-yl)methyl)urea

[M+H⁺]=463.1,

¹H NMR (400 MHz, DMSO-d₆) δ 13.57 (br s, 1H), 8.55 (s, 3H), 7.66-7.46 (m, 2H), 6.64 (s, 1H), 4.90 (s, 2H), 3.96 (s, 3H).

### Example 12. Preparation of 3-(3-chloro-4,5-difluorophenyl)-1-((5-(difluoromethyl)-1H-pyrazol-3-yl)methyl)-1-(2-methoxypyrimidin-5-yl)urea

[M+H⁺]=445.1,

¹H NMR (400 MHz, CDCl₃) δ 8.33 (s, 2H), 7.30 (ddd, J=2.7 Hz, 6.3 Hz, 11.5 Hz, 1H), 7.18 (dd, J=2.4 Hz, 5.2 Hz, 1H), 6.85-6.54 (m, 1H), 6.47 (br s, 1H), 6.31 (s, 1H), 4.73 (s, 2H), 4.05 (s, 3H).

### Example 13. Preparation of 3-(6-chloro-4,5-difluoropyridin-2-yl)-1-(2-methoxypyrimidin-5-yl)-1-((5-(trifluoromethyl)-1H-pyrazol-3-yl)methyl)urea

[M+H⁺]=463.9,

¹H NMR (400 MHz, CDCl₃) δ 11.76-10.67 (m, 1H), 8.30-8.26 (m, 2H), 7.96 (dd, J=4.8 Hz, 10.9 Hz, 1H), 6.77 (s, 1H), 6.30 (s, 1H), 4.70-4.65 (m, 2H), 4.06-4.01 (m, 3H).

### Example 14. Preparation of 3-(3-chloro-4-fluorophenyl)-1-(6-oxo-1,6-dihydropyridin-3-yl)-1-((4,5,6,7-tetrahydro-1H-indazol-3-yl)methyl)urea

¹H NMR (300 MHz, DMSO-d₆) δ 8.24 (s, 1H), 7.71 (dd, J=3.0 Hz, 1H) 7.44 (m, 1H), 7.31 (s, 1H), 7.28-7.16 (m, 2H), 6.29 (d, J=7.2 Hz, 1H), 4.46 (s, 2H), 2.28 (s, 2H), 1.65-1.64 (m, 4H).

### Example 15. Preparation of 3-(3-chloro-4-fluorophenyl)-1-(2-oxo-1,2-dihydropyridin-4-yl)-1-((4,5,6,7-tetrahydro-1H-indazol-3-yl)methyl)urea

[M+H⁺]=416.3

¹H NMR (500 MHz, Methanol-d₄) δ 7.66 (dd, J=6.6 Hz, 2.6 Hz, 1H), 7.40-7.31 (m, 2H), 7.16 (t, J=9.0 Hz, 1H), 6.44 (d, J=7.3 Hz, 1H), 6.32 (d, J=2.2 Hz, 1H), 4.92 (s, 2H), 2.59 (t, J=6.1 Hz, 2H), 2.45 (t, J=6.0 Hz, 2H), 1.87-1.65 (m, 4H).

### Example 16. Preparation of N-(4-(3-(3-chloro-4-fluorophenyl)-1-((1,4,5,7-tetrahydropyrano[3,4-c]pyrazol-3-yl)methyl)ureido)phenyl)methanesulfonamide

[M+H⁺]=494.1,

¹H NMR (400 MHz, DMSO-d₆) δ 9.81 (s, 1H), 8.13 (s, 1H), 7.71-7.69 (dd, J=2.4 Hz, 6.8 Hz, 1H), 7.40-7.37 (m, 1H), 7.25 (t, J=9.2 Hz, 1H), 7.19-7.14 (m, 4H), 4.78 (s, 2H), 4.54 (s, 2H), 3.67 (t, J=5.2 Hz, 2H), 3.00 (s, 3H), 2.32-2.31 (m, 2H).

### Example 17. Preparation of N-(5-(3-(3-chloro-4-fluorophenyl)-1-((1,4,5,7-tetrahydropyrano[3,4-c]pyrazol-3-yl)methyl)ureido)pyridin-2-yl)methanesulfonamide

[M+H⁺]=495.2,

¹H NMR (400 MHz, DMSO) δ 8.32 (s, 1H), 8.04 (s, 1H), 7.71-7.69 (m, 1H), 7.53-7.52 (m, 1H), 7.41-7.40 (m, 1H), 7.40-7.26 (m, 1H), 6.92-6.90 (m, 1H), 4.79 (s, 2H), 4.57 (s, 2H), 3.72-3.70 (m, 2H), 3.48-3.24 (m, 3H), 2.45-2.34 (m, 2H).

### Example 18. Preparation of N-(5-(3-(3-chloro-4-fluorophenyl)-1-((1,4,5,6-tetrahydropyrano[2,3-c]pyrazol-3-yl)methyl)ureido)pyridin-2-yl)methanesulfonamide

[M+H⁺]=495.2,

¹H NMR (400 MHz, CDCl₃) δ 8.18 (d, J=1.9 Hz, 1H), 7.53-7.48 (m, 1H), 7.44 (dd, J=2.6 Hz, 6.5 Hz, 1H), 7.20 (d, J=8.6 Hz, 1H), 7.17-7.12 (m, 1H), 7.06-6.99 (m, 1H), 6.41 (br s, 1H), 4.69 (s, 2H), 4.22-4.16 (m, 2H), 3.28 (s, 3H), 2.31 (br t, J=6.2 Hz, 2H), 1.91-1.82 (m, 2H).

### Example 19. Preparation of N-(4-(3-(3-chloro-4-fluorophenyl)-1-((1,4,5,6-tetrahydropyrano[2,3-c]pyrazol-3-yl)methyl)ureido)phenyl)methanesulfonamide

[M+H⁺]=494.2,

¹H NMR (400 MHz, CDCl₃) δ 8.57 (d, J=11.4 Hz, 1H), 8.36 (s, 1H), 7.74 (dd, J=2.8 Hz, 6.5 Hz, 1H), 7.42 (dd, J=2.6 Hz, 6.5 Hz, 1H), 7.32-7.29 (m, 1H), 7.28 (s, 1H), 7.20-7.17 (m, 1H), 7.17-7.15 (m, 1H), 7.13-7.09 (m, 1H), 7.05-6.99 (m, 1H), 6.91 (t, J=8.8 Hz, 1H), 6.69 (dd, J=2.8 Hz, 6.1 Hz, 1H), 6.50 (td, J=3.3 Hz, 8.8 Hz, 1H), 6.17 (s, 1H), 4.65 (s, 2H), 4.21-4.15 (m, 2H), 3.11 (s, 3H), 2.28-2.19 (m, 2H), 1.88-1.80 (m, 2H).

### Example 20. Preparation of 1-(6-acetylpyridin-3-yl)-3-(3-chloro-4-fluorophenyl)-1-((1,4,5,6-tetrahydropyrano[2,3-c]pyrazol-3-yl)methyl)urea

[M+H⁺]=444.2,

¹H NMR (400 MHz, CDCl₃) δ 8.58 (d, J=2.4 Hz, 1H), 8.11 (d, J=8.4 Hz, 1H), 7.69 (dd, J=2.5 Hz, 8.4 Hz, 1H), 7.46 (dd, J=2.6 Hz, 6.5 Hz, 1H), 7.17-7.11 (m, 1H), 7.09-7.01 (m, 1H), 6.58 (br s, 1H), 4.73 (s, 2H), 4.23-4.14 (m, 2H), 2.73 (s, 3H), 2.16 (t, J=6.3 Hz, 2H), 1.89-1.79 (m, 2H).

### Example 21. Preparation of 1-(6-acetylpyridin-3-yl)-3-(3-chloro-4-fluorophenyl)-1-((1,4,5,7-tetrahydropyrano[3,4-c]pyrazol-3-yl)methyl)urea

[M+H⁺]=444.3,

¹H NMR (400 MHz, DMSO) δ 12.45 (s, 1H), 8.88 (s, 1H), 8.63 (s, 1H), 7.98-7.95 (m, 1H), 7.72-7.70 (m, 2H), 7.39-7.34 (m, 1H), 7.32-7.29 (m, 1H), 4.97 (s, 2H), 4.58 (s, 2H), 3.72-3.69 (m, 2H), 2.52 (m, 3H), 2.51-2.43 (m, 2H).

### Example 22. Preparation of 1-(2-acetylpyridin-4-yl)-3-(3-chloro-4-fluorophenyl)-1-((1,4,5,7-tetrahydropyrano[3,4-c]pyrazol-3-yl)methyl)urea

[M+H⁺]=444.5,

¹H NMR (400 MHz, CDCl₃) δ 8.65 (d, J=5.4 Hz, 1H), 7.94 (d, J=2.1 Hz, 1H), 7.80 (s, 1H), 7.53 (dd, J=2.6 Hz, 6.4 Hz, 1H), 7.46 (dd, J=2.3 Hz, 5.4 Hz, 1H), 7.22 (td, J=3.4 Hz, 8.8 Hz, 1H), 7.12-7.05 (m, 1H), 4.90 (s, 2H), 4.71 (s, 2H), 3.83 (t, J=5.5 Hz, 2H), 2.73 (s, 3H), 2.42 (t, J=5.4 Hz, 2H).

### Example 23. Preparation of 4-(3-(3-chloro-4-fluorophenyl)-1-((1,4,5,7-tetrahydropyrano[3,4-c]pyrazol-3-yl)methyl)ureido)-N-methylbenzenesulfonamide

[M+H⁺]=494.0,

¹H NMR (400 MHz, CDCl₃) δ 8.55 (s, 1H), 7.93-7.86 (m, 2H), 7.60-7.54 (m, 1H), 7.49-7.45 (m, 2H), 7.37-7.30 (m, 1H), 7.22-7.13 (m, 1H), 4.95 (s, 2H), 4.60 (m, 2H), 3.71-3.63 (m, 2H), 2.54 (s, 3H), 2.49-2.31 (m, 2H), 1.75-1.67 (m, 3H).

### Example 24. Preparation of 3-(3-chloro-4-fluorophenyl)-1-(1-methyl-1H-pyrazol-4-yl)-1-((4,5,6,7-tetrahydro-1H-indazol-3-yl)methyl)urea

[M+H⁺]=405.1,

¹H NMR (500 MHz, CDCl₃) δ 7.48 (dd, J=6.5 Hz, 2.6 Hz, 1H), 7.37 (s, 2H), 7.14-7.08 (m, 1H), 6.99 (t, J=8.8 Hz, 2H), 4.69 (s, 2H), 3.86 (s, 3H), 2.60 (t, J=6.1 Hz, 2H), 2.31 (t, J=5.9 Hz, 2H), 1.76 (dd, J=10.6 Hz, 4.7 Hz, 2H), 1.68 (dd, J=10.4 Hz, 4.7 Hz, 2H).

### Example 25. Preparation of 3-(3-chloro-4-fluorophenyl)-1-(1-methyl-1H-pyrazol-3-yl)-1-((1,4,5,6-tetrahydropyrano[2,3-c]pyrazol-3-yl)methyl)urea

[M+H⁺]=405.0,

¹H NMR (300 MHz, DMSO) δ 11.56 (s, 1H), 10.37 (s, 1H), 7.89 (dd, J=6.8 Hz, 2.6 Hz, 1H), 7.71 (d, J=1.9 Hz, 1H), 7.47 (ddd, J=9.0 Hz, 4.3 Hz, 2.7 Hz, 1H), 7.36 (t, J=9.1 Hz, 1H), 6.04 (d, J=2.1 Hz, 1H), 4.87 (s, 2H), 4.03 (d, J=7.1 Hz, 2H), 3.85 (s, 3H), 2.32 (t, J=6.3 Hz, 2H), 1.79-1.67 (m, 2H).

### Example 26. Preparation of 3-(3-chloro-4-fluorophenyl)-1-(5-methylisoxazol-3-yl)-1-((1,4,5,6-tetrahydropyrano[2,3-c]pyrazol-3-yl)methyl)urea

[M+H⁺]=506,

¹H NMR (400 MHz, DMSO-d₆) δ 11.72 (s, 1H), 9.73 (s, 1H), 7.80 (dd, J=6.8 Hz, 2.6 Hz, 1H), 7.45 (ddd, J=9.1 Hz, 4.4 Hz, 2.6 Hz, 1H), 7.38 (t, J=9.0 Hz, 1H), 6.43 (s, 1H), 4.94 (s, 2H), 4.15-3.96 (m, 2H), 2.39 (s, 3H), 1.82-1.71 (m, 2H).

### Example 27. Preparation of 3-(3-chloro-4-fluorophenyl)-1-(3-methylbicyclo[1.1.1]pentan-1-yl)-1-((5-(trifluoromethyl)-1H-pyrazol-3-yl)methyl)urea

[M+H⁺]=416.9,

¹H NMR (400 MHz, CDCl₃) δ 11.61-10.85 (m, 1H), 7.53 (dd, J=2.6 Hz, 6.4 Hz, 1H), 7.25-7.18 (m, 1H), 7.13-7.07 (m, 1H), 6.75 (s, 1H), 6.40 (s, 1H), 4.45 (s, 2H), 2.09 (s, 6H), 1.32 (s, 3H).

### Example 28. Preparation of 3-(3-chloro-4-fluorophenyl)-1-(2-methoxy-4-methylpyrimidin-5-yl)-1-((5-(trifluoromethyl)-1H-pyrazol-3-yl)methyl)urea

[M+H⁺]=459.1,

¹H NMR (400 MHz, DMSO-d₆) δ 13.49 (s, 1H), 8.31 (s, 1H), 8.26 (s, 1H), 7.71 (dd, J=2.5 Hz, 6.9 Hz, 1H), 7.45-7.38 (m, 1H), 7.33-7.26 (m, 1H), 6.61 (s, 1H), 5.11-4.51 (m, 2H), 3.94 (s, 3H), 2.13 (s, 3H).

### Example 29. Preparation of 3-(3-chloro-4-fluorophenyl)-1-(4-methoxy-2,6-dimethylphenyl)-1-((5-(trifluoromethyl)-1H-pyrazol-3-yl)methyl)urea

[M+H⁺]=471.1,

¹H NMR (400 MHz, DMSO-d₆) δ 13.41 (s, 1H), 7.93-7.78 (m, 1H), 7.73 (dd, J=2.6 Hz, 6.8 Hz, 1H), 7.44 (ddd, J=2.8 Hz, 4.2 Hz, 9.1 Hz, 1H), 7.25 (t, J=9.1 Hz, 1H), 6.74 (s, 2H), 6.49 (s, 1H), 4.66 (s, 2H), 3.34 (s, 2H), 1.92 (s, 6H).

### Example 30. Preparation of 3-(3-chloro-4-fluorophenyl)-1-(2-methoxy-4,6-dimethylpyrimidin-5-yl)-1-((5-(trifluoromethyl)-1H-pyrazol-3-yl)methyl)urea

[M+H⁺]=473.1,

¹H NMR (400 MHz, Chloroform-d) δ 11.59 (s, 1H), 7.50 (dd, J=2.7 Hz, 6.4 Hz, 1H), 7.21-7.15 (m, 2H), 7.00 (t, J=8.8 Hz, 1H), 6.68 (s, 1H), 6.25 (s, 1H), 4.55 (s, 2H), 3.91 (s, 3H), 2.09 (s, 6H).

### Example 31. Preparation of methyl

4-(3-(3-chloro-4-fluorophenyl)-1-((4,5,6,7-tetrahydro-1H-indazol-3-yl)methyl)ureido)benzoate

[M+H⁺]=457.3,

¹H NMR (500 MHz, DMSO-d₆) δ 12.19 (s, 1H), 8.81 (s, 1H), 7.92 (d, J=8.6 Hz, 2H), 7.71 (dd, J=6.8 Hz, 2.6 Hz, 1H), 7.43 (d, J=8.7 Hz, 2H), 7.39 (ddd, J=6.9 Hz, 4.3 hz, 2.2 Hz, 1H), 7.31 (t, J=9.1 Hz, 1H), 4.86 (s, 2H), 3.84 (s, 3H), 2.50 (d, J=2.0 Hz, 2H), 2.29 (t, J=6.0 Hz, 2H), 1.73-1.54 (m, 4H).

### Example 32. Preparation of 3-(3-chloro-4-fluorophenyl)-1-(4-methoxyphenyl)-1-((4,5,6,7-tetrahydro-1H-indazol-3-yl)methyl)urea

[M+H⁺]=431.05,

¹H NMR (300 MHz, Chloroform-d) δ 7.44 (dd, J=6.5 Hz, 2.6 Hz, 1H), 7.14-7.05 (m, 3H), 7.00 (t, J=8.8 Hz, 1H), 6.96-6.90 (m, 2H), 6.27 (s, 1H), 4.70 (s, 2H), 3.84 (s, 3H), 2.64 (t, J=6.2 Hz, 2H), 2.20 (t, J=6.0 Hz, 2H), 1.77 (dt, J=10.6 Hz, 5.9 Hz, 2H), 1.71-1.59 (m, 2H).

### Example 33. Preparation of 1-((5-acetyl-4,5,6,7-tetrahydro-1H-pyrazolo[4,3-c]pyridin-3-yl)methyl)-3-(3-chloro-4-fluorophenyl)-1-(4-methoxyphenyl)urea

[M+H⁺]=472.2,

¹H NMR (500 MHz, DMSO-d₆) δ 12.37-12.29 (m, 1H), 8.06, 8.00 (s, 1H), 7.73-7.71 (m, 1H), 7.44-7.40 (m, 1H), 7.28-7.24 (m, 1H), 7.17-7.13 (m, 2H), 6.94 (t, J=11.1 Hz, 2H), 4.78, 4.74 (s, 2H), 4.38 (d, J=8.40 Hz, 1H), 4.27, 4.23 (s, 1H), 3.77, 3.76 (s, 3H), 3.68-3.61 (m, 2H), 2.70-2.50 (m, 2H), 2.08-1.93 (m, 3H).

### Example 34. Preparation of 1-((1H-pyrazol-3-yl)methyl)-3-(3-chloro-4-fluorophenyl)-1-(4-methoxyphenyl)urea

[M+H⁺]=375,

¹H NMR (300 MHz, Chloroform-d) δ 7.52 (d, J=1.7 Hz, 1H), 7.47 (dd, J=6.5 Hz, 2.6 Hz, 1H), 7.12 (dd, J=9.3 Hz, 2.8 Hz, 3H), 7.03 (t, J=8.7 Hz, 1H), 6.97 (d, J=8.9 Hz, 2H), 6.20 (s, 1H), 6.18 (s, 1H), 4.81 (s, 2H), 3.87 (s, 3H).

### Example 35. Preparation of 3-(3-chloro-4-fluorophenyl)-1-(4-methoxyphenyl)-1-((1-methyl-1H-pyrazol-3-yl)methyl)urea

[M+H⁺]=389,

¹H NMR (300 MHz, Chloroform-d) δ 7.53 (dd, J=6.5 Hz, 2.6 Hz, 1H), 7.30 (d, J=2.2 Hz, 1H), 7.17 (d, J=8.9 Hz, 2H), 7.13 (dt, J=4.0 Hz, 2.3 Hz, 1H), 7.01 (t, J=8.8 Hz, 1H), 6.94 (d, J=8.9 Hz, 2H), 6.43 (s, 1H), 6.28 (d, J=2.2 Hz, 1H), 4.86 (s, 2H), 3.85 (d, J=1.6 Hz, 6H).

### Example 36. Preparation of 3-(3-chloro-4-fluorophenyl)-1-(4-methoxyphenyl)-1-((1-methyl-4,5,6,7-tetrahydro-1H-indazol-3-yl)methyl)urea

¹H NMR (300 MHz, CDCl₃) δ 7.43 (dd, J=6.5 Hz, 2.6 Hz, 1H), 7.14-7.05 (m, 1H), 7.01 (d, J=8.6 Hz, 1H), 6.97-6.87 (m, 4H), 6.06 (s, 1H), 4.86 (s, 2H), 3.84 (s, 3H), 3.72 (s, 3H), 2.57 (t, J=6.4 Hz, 2H), 1.92 (t, J=6.0 Hz, 2H), 1.72-1.60 (m, 2H), 1.54-1.47 (m, 2H).

### Example 37. Preparation of 3-(3-chloro-4-fluorophenyl)-1-(4-methoxyphenyl)-1-((5-methyl-4,5,6,7-tetrahydro-1H-pyrazolo[4,3-c]pyridin-3-yl)methyl)urea

[M+H⁺]=444.6,

¹H NMR (500 MHz, DMSO-d₆) δ 12.20 (s, 1H), 8.02 (s, 1H), 7.70 (dd, J=7.2 Hz, 2.6 Hz, 1H), 7.41 (ddd, J=9.1 Hz, 4.3 Hz, 2.6 Hz, 1H), 7.27 (t, J=9.1 Hz, 1H), 7.11 (d, J=8.9 Hz, 2H), 6.94 (d, J=8.7 Hz, 2H), 4.71 (s, 2H), 3.76 (s, 3H), 3.26 (s, 2H), 2.63 (s, 4H), 2.34 (s, 3H).

### Example 38. Preparation of 3-(3-chloro-4-fluorophenyl)-1-(4-methoxyphenyl)-1-((1,4,6,7-tetrahydropyrano[4,3-c]pyrazol-3-yl)methyl)urea

[M+H⁺]=431.1,

¹H NMR (300 MHz, CDCl₃) δ 7.43 (dd, J=6.5 Hz, 2.5 Hz, 1H), 7.14-7.02 (m, 3H), 6.95 (dd, J=19.6 Hz, 8.8 Hz, 3H), 6.29 (s, 1H), 4.70 (s, 2H), 4.38 (s, 2H), 3.86 (t, J=5.6 Hz, 2H), 3.82 (s, 3H), 3.43 (s, 1H), 2.75 (t, J=5.5 Hz, 2H).

### Example 39. Preparation of 3-(3-chloro-4-fluorophenyl)-1-(4-methoxyphenyl)-1-((1-methyl-1,4,6,7-tetrahydropyrano[4,3-c]pyrazol-3-yl)methyl)urea

[M+H⁺]=445.3,

¹H NMR (500 MHz, DMSO-d₆) δ 8.01 (s, 1H), 7.69 (dd, J=6.9 Hz, 2.6 Hz, 1H), 7.46-7.34 (m, 1H), 7.26 (t, J=9.1 Hz, 1H), 7.14 (d, J=8.9 Hz, 2H), 6.95 (d, J=8.9 Hz, 2H), 4.67 (s, 2H), 4.43 (s, 2H), 3.79-3.76 (m, 5H), 3.60 (s, 3H), 2.64 (t, J=5.6 Hz, 2H).

### Example 40. Preparation of 3-(3-chloro-4-fluorophenyl)-1-((5,5-difluoro-4,5,6,7-tetrahydro-1H-indazol-3-yl)methyl)-1-(6-methoxypyridin-3-yl)urea

[M+H⁺]=466.2,

¹H NMR (500 MHz, DMSO-d₆) δ 12.35 (s, 1H), 8.20 (s, 1H), 7.96 (d, J=2.7 Hz, 1H), 7.69 (dd, J=6.9 Hz, 2.6 Hz, 1H), 7.53 (dd, J=8.8 Hz, 2.7 Hz, 1H), 7.45-7.41 (m, 1H), 7.28 (t, J=9.1 Hz, 1H), 6.84 (d, J=8.7 Hz, 1H), 4.73 (s, 2H), 3.86 (s, 3H), 2.92 (t, J=14.4 Hz, 2H), 2.74 (t, J=6.8 Hz, 2H), 2.25-2.19 (dt, J=13.9 Hz, 7.1 Hz, 2H).

### Example 41. Preparation of 3-(3-chloro-4-fluorophenyl)-1-(6-methoxypyridin-3-yl)-1-((1,4,5,6-tetrahydrocyclopenta[c]pyrazol-3-yl)methyl)urea

[M+H⁺]=418.2,

¹H NMR (300 MHz, CDCl₃) δ 8.02 (d, J=2.6 Hz, 1H), 7.44 (dd, J=6.5 Hz, 2.5 Hz, 1H), 7.34 (dd, J=8.7 Hz, 2.6 Hz, 1H), 7.27 (s, 1H), 7.15-7.06 (m, 1H), 6.99 (t, J=8.7 Hz, 1H), 6.78 (d, J=8.8 Hz, 1H), 6.46 (s, 1H), 4.70 (s, 2H), 3.95 (s, 3H), 2.67 (t, J=6.9 Hz, 2H), 2.51-2.27 (m, 4H), 2.16 (s, 1H).

### Example 42. Preparation of 3-(3-chloro-4-fluorophenyl)-1-(6-methoxypyridin-3-yl)-1-((4,5,6,7-tetrahydro-1H-indazol-3-yl)methyl)urea

[M+H⁺]=431.19,

¹H NMR (500 MHz, MeOD) δ 7.83 (d, J=2.6 Hz, 1H), 7.57 (dd, J=6.7 Hz, 2.6 Hz, 1H), 7.43 (dd, J=8.8 Hz, 2.7 Hz, 1H), 7.25 (ddd, J=9.0 Hz, 4.1 Hz, 2.7 Hz, 1H), 7.10 (t, J=9.0 Hz, 1H), 6.80 (d, J=8.9 Hz, 1H), 4.82 (s, 2H), 3.90 (s, 3H), 2.59 (t, J=6.1 Hz, 2H), 2.33 (s, 2H), 1.81-1.74 (m, 2H), 1.70 (d, J=5.5 Hz, 2H).

### Example 43. Preparation of 1-((1H-indazol-3-yl)methyl)-3-(3-chloro-4-fluorophenyl)-1-(6-methoxypyridin-3-yl)urea

[M+H⁺]=426.1,

¹H NMR (500 MHz, DMSO-d₆) δ 12.82 (s, 1H), 8.25 (s, 1H), 7.85 (s, 1H), 7.80 (s, 1H), 7.73 (d, J=6.8 Hz, 1H), 7.51-7.40 (m, 3H), 7.35 (t, J=7.6 Hz, 1H), 7.29 (t, J=8.7 Hz, 1H), 7.12 (t, J=7.5 Hz, 1H), 6.79 (d, J=8.7 Hz, 1H), 5.19 (s, 2H), 3.82 (s, 3H).

### Example 44. Preparation of 3-(3-chloro-4-fluorophenyl)-1-((5-(hydroxymethyl)-4,5,6,7-tetrahydro-1H-indazol-3-yl)methyl)-1-(6-methoxypyridin-3-yl)urea

[M+H⁺]=460.2,

¹H NMR (500 MHz, DMSO-d₆) δ 12.08 (s, 1H), 8.22 (s, 1H), 7.92 (s, 1H), 7.69 (d, J=6.4 Hz, 1H), 7.50 (d, J=8.5 Hz, 1H), 7.46-7.33 (m, 1H), 7.27 (t, J=9.1 Hz, 1H), 6.82 (d, J=8.6 Hz, 1H), 4.77 (d, J=15.2 Hz, 1H), 4.68 (d, J=15.2 Hz, 1H), 4.52 (t, J=5.2 Hz, 1H), 3.85 (s, 3H), 3.36-3.30 (m, 2H), 2.63-2.55 (m, 1H), 2.49-2.40 (m, 2H), 1.92-1.85 (m, 2H), 1.72-1.63 (m, 1H), 1.33-1.25 (m, 1H).

### Example 45. Preparation of 3-(3-chloro-4-fluorophenyl)-1-(6-methoxypyridin-3-yl)-1-((1,4,6,7-tetrahydrothiopyrano[4,3-c]pyrazol-3-yl)methyl)urea

[M+H⁺]=448.3,

¹H NMR (500 MHz, DMSO-d₆) δ 12.29 (s, 1H), 8.19 (s, 1H), 7.97 (t, J=2.1 Hz, 1H), 7.69 (d, J=6.8 Hz, 1H), 7.55 (d, J=8.8 Hz, 1H), 7.48-7.36 (m, 1H), 7.27 (t, J=9.1 Hz, 1H), 6.83 (d, J=8.7 Hz, 1H), 4.75 (s, 2H), 3.86 (s, 3H), 3.51 (s, 2H), 2.97 J 2.61 (m, 4H).

### Example 46. Preparation of 3-(3-chloro-4-fluorophenyl)-1-(4-methoxyphenyl)-1-((1,4,5,6-tetrahydrocyclopenta[c]pyrazol-3-yl)methyl)urea

[M+H⁺]=417.2,

¹H NMR (300 MHz, CDCl₃) δ 7.44 (dd, J=6.5 Hz, 2.6 Hz, 1H), 7.16-7.08 (m, 3H), 7.06-6.95 (m, 3H), 6.17 (s, 1H), 4.69 (s, 2H), 3.87 (s, 3H), 2.72 (t, J=7.4 Hz, 2H), 2.39 (dd, J=9.4 Hz, 4.0 Hz, 4H).

### Example 47. Preparation of 3-(3-chloro-4-fluorophenyl)-1-((5,5-dimethyl-4,5,6,7-tetrahydro-1H-indazol-3-yl) methyl)-1-(6-methoxypyridin-3-yl)urea

[M+H⁺]=458.19,

¹H NMR (300 MHz, CDCl₃) δ 8.01 (s, 1H), 7.53-7.46 (m, 1H), 7.35 (d, J=8.7 Hz, 1H), 7.15 (s, 1H), 7.04 (t, J=8.7 Hz, 1H), 6.80 (d, J=8.8 Hz, 1H), 6.55 (s, 1H), 4.73 (d, J=1.6 Hz, 2H), 3.98 (d, J=2.1 Hz, 3H), 2.64 (s, 2H), 1.98 (s, 2H), 1.57 (d, J=6.1 Hz, 2H), 0.92 (d, J=1.8 Hz, 6H).

### Example 48. Preparation of 3-(3-chloro-4-fluorophenyl)-1-((5-hydroxy-4,5,6,7-tetrahydro-1H-indazol-3-yl)methyl)-1-(6-methoxypyridin-3-yl)urea

[M+H⁺]=446.24,

¹H NMR (500 MHz, CDCl₃) δ 8.00 (s, 1H), 7.46 (d, J=6.4 Hz, 1H), 7.38 (d, J=8.6 Hz, 1H), 7.12 (d, J=9.0 Hz, 1H), 7.01 (t, J=8.5 Hz, 1H), 6.80 (d, J=8.6 Hz, 1H), 6.40 (s, 1H), 4.75-4.67 (m, 2H), 4.10 (s, 1H), 3.96 (s, 3H), 2.82 (dd, J=14.6 Hz, 8.3 Hz, 1H), 2.72-2.64 (m, 1H), 2.60 (dd, J=15.5 Hz, 4.5 Hz, 1H), 2.26 (dd, J=15.4 Hz, 6.8 Hz, 1H), 1.95 (s, 1H), 1.92-1.86 (m, 1H).

### Example 49. Preparation of 3-(3-chloro-4-fluorophenyl)-1-((5,5-dioxido-1,4,6,7-tetrahydrothiopyrano[4,3-c]pyrazol-3-yl)methyl)-1-(6-methoxypyridin-3-yl)urea

[M+H⁺]=480.3,

¹H NMR (500 MHz, DMSO-d₆) δ 12.65 (s, 1H), 8.19 (s, 1H), 8.01 (d, J=2.7 Hz, 1H), 7.68 (d, J=6.9 Hz, 1H), 7.58 (d, J=8.7 Hz, 1H), 7.49-7.38 (m, 1H), 7.29 (t, J=9.1 Hz, 1H), 6.85 (d, J=8.7 Hz, 1H), 4.72 (s, 2H), 4.19 (s, 2H), 3.86 (s, 3H), 3.39 (t, J=6.5 Hz, 2H), 3.10 (d, J=6.6 Hz, 2H).

### Example 50. Preparation of 3-(3-chloro-4-fluorophenyl)-1-(6-methoxypyridin-3-yl)-1-((5-methyl-1H-pyrazol-3-yl)methyl)urea

[M+H⁺]=390.14,

¹H NMR (300 MHz, CDCl₃) δ 8.04 (d, J=2.6 Hz, 1H), 7.48 (dd, J=6.5 Hz, 2.6 Hz, 1H), 7.41 (dd, J=8.8 Hz, 2.7 Hz, 1H), 7.16-7.08 (m, 1H), 7.01 (t, J=8.7 Hz, 1H), 6.79 (d, J=8.8 Hz, 1H), 6.46 (s, 1H), 5.96 (s, 1H), 4.74 (s, 2H), 3.96 (s, 3H), 2.27 (s, 3H).

### Example 51. Preparation of 3-(3-chloro-4-fluorophenyl)-1-(6-methoxypyridin-3-yl)-1-((4,5,6,7-tetrahydro-1H-4,7-methanoindazol-3-yl)methyl)urea

[M+H⁺]=442.3,

¹H NMR (500 MHz, DMSO-d₆) δ 11.61 (s, 1H), 8.28 (s, 1H), 7.94 (s, 1H), 7.72 (d, J=6.8 Hz, 1H), 7.45 (t, J=12.3 Hz, 2H), 7.29 (t, J=9.1 Hz, 1H), 6.85 (d, J=8.7 Hz, 1H), 4.90-4.64 (m, 2H), 3.86 (s, 3H), 3.18 (s, 1H), 3.05 (s, 1H), 1.91-1.63 (m, 3H), 1.54 (d, J=8.5 Hz, 1H), 1.01 (s, 1H), 0.86 (t, J=10.1 Hz, 1H).

### Example 52. Preparation of 3-((3-(3-chloro-4-fluorophenyl)-1-(6-methoxypyridin-3-yl)ureido)methyl)-4,5,6,7-tetrahydro-1H-indazole-5-carboxylic acid

[M+H⁺]=474.3.

### Example 53. Preparation of methyl 3-((3-(3-chloro-4-fluorophenyl)-1-(6-methoxypyridin-3-yl)ureido)methyl)-4,5,6,7-tetrahydro-1H-indazole-5-carboxylate

[M+H⁺]=488.2,

¹H NMR (500 MHz, DMSO-d₆) δ 12.15 (s, 1H), 8.21 (s, 1H), 7.93 (s, 1H), 7.68 (s, 1H), 7.51 (d, J=8.5 Hz, 1H), 7.45-7.35 (m, 1H), 7.28 (t, J=9.1 Hz, 1H), 6.82 (d, J=8.7 Hz, 1H), 4.82 (d, J=14.9 Hz, 1H), 4.64 (d, J=15.3 Hz, 1H), 3.86 (s, 3H), 3.62 (s, 3H), 2.76-2.53 (m, 4H), 2.49-2.32 (m, 1H), 2.06 (d, J=13.0 Hz, 1H), 1.74 (s, 1H).

### Example 54. Preparation of 3-(3-chloro-4-fluorophenyl)-1-(2-methoxypyridin-4-yl)-1-((4,5,6,7-tetrahydro-1H-indazol-3-yl)methyl)urea

[M+H⁺]=432.0,

¹H NMR (300 MHz, CDCl₃) δ 8.11 (d, J=5.6 Hz, 1H), 7.93 (s, 1H), 7.53 (dd, J=6.5 Hz, 2.6 Hz, 1H), 7.25-7.17 (m, 1H), 7.02 (t, J=8.8 Hz, 1H), 6.83 (dd, J=5.6 Hz, 1.7 Hz, 1H), 6.65 (d, J=1.6 Hz, 1H), 4.82 (s, 2H), 3.93 (s, 3H), 2.72-2.47 (m, 2H), 2.25 (t, J=5.9 Hz, 2H), 1.72 (ddd, J=15.5 Hz, 9.6 Hz, 3.6 Hz, 4H).

### Example 55. Preparation of 3-(3-chloro-4-fluorophenyl)-1-(6-methoxypyridin-3-yl)-1-((1,4,6,7-tetrahydropyrano[4,3-c]pyrazol-3-yl)methyl)urea

[M+H⁺]=432.2,

¹H NMR (500 MHz, CDCl₃) δ 8.02 (d, J=2.6 Hz, 1H), 7.45 (dd, J=6.4 Hz, 2.7 Hz, 1H), 7.35 (dd, J=8.7 Hz, 2.7 Hz, 1H), 7.15-7.09 (m, 1H), 7.02 (t, J=8.7 Hz, 1H), 6.82 (d, J=8.8 Hz, 1H), 6.17 (s, 1H), 4.68 (s, 2H), 4.47 (s, 2H), 3.97 (s, 3H), 3.90 (t, J=5.6 Hz, 2H), 2.79 (t, J=5.6 Hz, 2H).

### Example 56. Preparation of 3-(3-chloro-4-fluorophenyl)-1-(6-methoxypyridin-3-yl)-1-((4-methyl-1H-pyrazol-3-yl)methyl)urea

[M+H⁺]=390.2,

¹H NMR (500 MHz, DMSO) δ 8.20 (s, 1H), 7.93-7.87 (m, 1H), 7.70 (dd, J=6.9 Hz, 2.6 Hz, 1H), 7.48 (dd, J=8.7 Hz, 2.7 Hz, 1H), 7.42 (ddd, J=9.1 Hz, 4.3 Hz, 2.7 Hz, 1H), 7.35 (s, 1H), 7.27 (t, J=9.1 Hz, 1H), 6.81 (d, J=8.8 Hz, 1H), 4.79 (s, 2H), 3.84 (s, 3H), 1.88 (s, 3H).

### Example 57. Preparation of 3-(3-chloro-4-fluorophenyl)-1-((4,5-dimethyl-1H-pyrazol-3-yl)methyl)-1-(6-methoxypyridin-3-yl)urea

[M+H⁺]=406.3,

¹H NMR (500 MHz, DMSO) δ 12.09 (s, 1H), 8.22 (s, 1H), 7.91 (d, J=2.6 Hz, 1H), 7.70 (dd, J=6.8 Hz, 2.3 Hz, 1H), 7.49 (dd, J=8.6 Hz, 2.3 Hz, 1H), 7.42 (ddd, J=9.1 Hz, 4.3 Hz, 2.7 Hz, 1H), 7.27 (t, J=9.1 Hz, 1H), 6.81 (d, J=8.8 Hz, 1H), 4.74 (s, 2H), 3.84 (s, 3H), 2.06 (s, 3H), 1.80 (s, 3H).

### Example 58. Preparation of 3-(3-chloro-4-fluorophenyl)-1-((5-isopropyl-1H-pyrazol-3-yl)methyl)-1-(6-methoxypyridin-3-yl)urea

[M+H⁺]=420.3,

¹H NMR (500 MHz, DMSO) δ 12.29 (s, 1H), 8.29 (s, 1H), 8.00 (d, J=2.5 Hz, 1H), 7.72 (dd, J=6.8 Hz, 2.5 Hz, 1H), 7.56 (dd, J=8.7 Hz, 2.7 Hz, 1H), 7.43 (ddd, J=9.1 Hz, 4.2 Hz, 2.7 Hz, 1H), 7.28 (t, J=9.1 Hz, 1H), 6.83 (d, J=8.8 Hz, 1H), 5.92 (s, 1H), 4.71 (s, 2H), 3.85 (s, 3H), 2.88 (s, 1H), 1.17 (d, J=6.7 Hz, 6H).

### Example 59. Preparation of 3-(3-chloro-4-fluorophenyl)-1-(6-methoxypyridin-3-yl)-1-((6-methyl-1,4,5,6-tetrahydrocyclopenta[c]pyrazol-3-yl)methyl)urea

[M+H⁺]=430.31,

¹H NMR (500 MHz, CDCl₃) δ 8.04 (dd, J=2.7 Hz, 0.5 Hz, 1H), 7.45 (dd, J=6.5 Hz, 2.7 Hz, 1H), 7.34 (dd, J=8.8 Hz, 2.7 Hz, 1H), 7.11 (ddd, J=8.9 Hz, 4.0 Hz, 2.7 Hz, 1H), 7.03 (dd, J=14.9 Hz, 6.2 Hz, 1H), 6.82 (dd, J=8.8 Hz, 0.6 Hz, 1H), 6.17 (s, 1H), 4.72-4.63 (m, 2H), 3.98 (s, 3H), 3.11 (dd, J=14.4 Hz, 6.9 Hz, 1H), 2.63-2.55 (m, 1H), 2.38-2.27 (m, 2H), 1.97-1.90 (m, 1H), 1.27 (d, J=6.9 Hz, 3H).

### Example 60. Preparation of 3-(3-chloro-4-fluorophenyl)-1-((5-ethyl-1H-pyrazol-3-yl)methyl)-1-(6-methoxypyridin-3-yl)urea

[M+H⁺]=404.1,

¹H NMR (400 MHz, CDCl₃) δ 8.05 (d, J=2.4 Hz, 1H), 7.50 (dd, J=6.5 Hz, 2.6 Hz, 1H), 7.42 (dd, J=8.8 Hz, 2.7 Hz, 1H), 7.18-7.10 (m, 1H), 7.02 (t, J=8.8 Hz, 1H), 6.81 (d, J=8.7 Hz, 1H), 6.41 (s, 1H), 6.01 (s, 1H), 4.76 (s, 2H), 3.97 (s, 3H), 2.66 (q, J=7.6 Hz, 2H), 1.26 (t, J=7.6 Hz, 3H).

### Example 61. Preparation of 1-((4-bromo-1H-pyrazol-3-yl)methyl)-3-(3-chloro-4-fluorophenyl)-1-(4-methoxyphenyl)urea

[M+H⁺]=456.23,

¹H NMR (400 MHz, CDCl₃) δ 8.07 (d, J=2.5 Hz, 1H), 7.50 (s, 1H), 7.47 (dd, J=6.4 Hz, 2.6 Hz, 1H), 7.37 (d, J=11.4 Hz, 1H), 7.12-7.08 (m, 1H), 7.04 (d, J=8.5 Hz, 1H), 7.00 (d, J=6.2 Hz, 1H), 6.83 (d, J=8.8 Hz, 1H), 6.12 (s, 1H), 4.77 (s, 2H), 3.98 (s, 3H).

### Example 62. Preparation of 3-(3-chloro-4-fluorophenyl)-1-(6-methoxypyridin-3-yl)-1-((5-(trifluoromethyl)-4,5,6,7-tetrahydro-1H-indazol-3-yl)methyl)urea

[M+H⁺]=498.36,

¹H NMR (500 MHz, CDCl₃) δ 8.00 (s, 1H), 7.45 (d, J=2.5 Hz, 1H), 7.37 (dd, J=5.8 Hz, 2.8 Hz, 1H), 7.11 (s, 1H), 7.03 (dd, J=8.5 Hz, 3.2 Hz, 1H), 6.85-6.79 (m, 1H), 6.25 (s, 1H), 4.71 (s, 2H), 3.99-3.94 (m, 3H), 2.88 (d, J=16.4 Hz, 1H), 2.66 (s, 1H), 2.50 (d, J=14.7 Hz, 1H), 2.32 (s, 1H), 2.25-2.19 (m, 2H), 1.74-1.67 (m, 1H).

### Example 63. Preparation of 3-(3-chloro-4-fluorophenyl)-1-(6-methoxypyridin-3-yl)-1-((1,4,5,6-tetrahydropyrano[2,3-c]pyrazol-3-yl)methyl)urea

[M+H⁺]=432.3,

¹H NMR (300 MHz, DMSO-d₆) δ 11.52 (s, 1H), 8.19 (s, 1H), 7.96 (dd, J=2.7 Hz, 0.7 Hz, 1H), 7.72 (dd, J=6.9 Hz, 2.6 Hz, 1H), 7.50 (dd, J=8.8 Hz, 2.7 Hz, 1H), 7.43 (ddd, J=9.1 Hz, 4.4 Hz, 2.6 Hz, 1H), 7.28 (t, J=9.1 Hz, 1H), 6.85 (d, J=8.7 Hz, 1H), 4.71 (s, 2H), 4.14-3.98 (m, 2H), 3.87 (s, 3H), 2.27 (t, J=6.3 Hz, 2H), 1.73 (t, J=4.83 Hz, 2H).

### Example 64. Preparation of 3-(3-chloro-4-fluorophenyl)-1-((5-methoxy-1H-pyrazol-3-yl)methyl)-1-(6-methoxypyridin-3-yl)urea

[M+H⁺]=406.1,

¹H NMR (300 MHz, MeOD) δ 7.98 (d, J=2.6 Hz, 1H), 7.60 (dd, J=6.7 Hz, 2.6 Hz, 1H), 7.50 (dd, J=8.8 Hz, 2.7 Hz, 1H), 7.27 (ddd, J=9.0 Hz, 4.2 Hz, 2.7 Hz, 1H), 7.11 (t, J=9.0 Hz, 1H), 6.85 (d, J=8.8 Hz, 1H), 5.65 (s, 1H), 4.78 (s, 2H), 3.93 (s, 3H), 3.81 (s, 3H).

### Example 65. Preparation of 3-(3-chloro-4-fluorophenyl)-1-(1-methyl-6-oxo-1,6-dihydropyridin-3-yl)-1-((1,4,5,6-tetrahydrocyclopenta[c]pyrazol-3-yl)methyl)urea

[M+H⁺]=418.0,

¹H NMR (300 MHz, MeOD) δ 7.62 (d, J=2.8 Hz, 1H), 7.59 (dd, J=6.7 Hz, 2.6 Hz, 1H), 7.29 (ddd, J=9.0 Hz, 4.2 Hz, 2.7 Hz, 1H), 7.23 (dd, J=9.5 Hz, 2.8 Hz, 1H), 7.11 (t, J=9.0 Hz, 1H), 6.51 (d, J=9.5 Hz, 1H), 4.74 (s, 2H), 3.52 (s, 3H), 2.66 (t, J=6.9 Hz, 2H), 2.56-2.35 (m, 4H).

### Example 66. Preparation of 3-(3-chloro-4-fluorophenyl)-1-(6-methoxypyridin-3-yl)-1-((1-(2-(tetrahydro-2H-pyran-2-yloxy)ethyl)-1,4,5,6-tetrahydrocyclopenta[c]pyrazol-3-yl)methyl)urea

[M+H⁺]=544.2,

¹H NMR (300 MHz, CDCl₃) δ 8.01 (d, J=2.4 Hz, 1H), 7.55 (dd, J=6.5 Hz, 2.6 Hz, 1H), 7.45 (dd, J=8.8 Hz, 2.7 Hz, 1H), 7.19 (ddd, J=8.9 Hz, 4.1 Hz, 2.7 Hz, 1H), 7.01 (t, J=8.8 Hz, 1H), 6.77 (d, J=8.8 Hz, 1H), 4.72 (s, 2H), 4.51 (d, J=3.7 Hz, 1H), 4.14 (dd, J=7.7 Hz, 3.4 Hz, 2H), 4.04-3.96 (m, 1H), 3.94 (s, 3H), 3.66 (ddd, J=11.4 Hz, 9.7 Hz, 4.6 Hz, 2H), 3.43 (dd, J=10.4 Hz, 5.6 Hz, 1H), 2.73 (d, J=4.9 Hz, 2H), 2.52 (d, J=4.0 Hz, 4H), 1.70 (dd, J=21.3 Hz, 10.6 Hz, 2H), 1.51 (dd, J=9.2 Hz, 3.4 Hz, 4H).

### Example 67. Preparation of 3-(3-chloro-4-fluorophenyl)-1-((1-(2-hydroxyethyl)-1,4,5,6-tetrahydrocyclopenta[c]pyrazol-3-yl)methyl)-1-(6-methoxypyridin-3-yl)urea

[M+H⁺]=460.3,

¹H NMR (300 MHz, CDCl₃) δ 7.98 (d, J=2.5 Hz, 1H), 7.51 (ddd, J=11.5 Hz, 7.6 Hz, 2.7 Hz, 2H), 7.17 (ddd, J=8.9 Hz, 4.0 Hz, 2.7 Hz, 1H), 7.03 (t, J=8.8 Hz, 1H), 6.82 (d, J=8.8 Hz, 1H), 6.63 (s, 1H), 4.76 (s, 2H), 4.07-4.01 (m, 2H), 3.97 (s, 3H), 3.92 (d, J=4.6 Hz, 2H), 2.70 (t, J=6.6 Hz, 2H), 2.62-2.52 (m, 4H).

### Example 68. Preparation of 3-(3-chloro-4-fluorophenyl)-1-((1,3a,4,5,6,7a-hexahydropyrano[2,3-c]pyrazol-3-yl)methyl)-1-(2-methoxypyridin-4-yl)urea

The title compound was synthesized in a similar manner as in Example 63 above.

### Example 69. Preparation of 3-(3-chloro-4-fluorophenyl)-1-((4,6-dihydro-1H-furo[3,4-c]pyrazol-3-yl)methyl)-1-(6-methoxypyridin-3-yl)urea

[M+H⁺]=418.2,

¹H NMR (400 MHz, Chloroform-d) δ 8.07-8.04 (m, 1H), 7.48-7.44 (m, 1H), 7.37-7.32 (m, 1H), 7.13-7.08 (m, 1H), 7.07-7.01 (m, 1H), 6.87-6.83 (m, 1H), 6.08-6.05 (m, 1H), 4.87-4.83 (m, 2H), 4.70-4.68 (m, 2H), 4.68-4.64 (m, 2H), 4.01-3.97 (m, 3H).

### Example 70. Preparation of 3-(3-chloro-4-fluorophenyl)-1-((4,6-dihydro-1H-furo[3,4-c]pyrazol-3-yl)methyl)-1-(2-methoxypyridin-4-yl)urea

### Preparation of 4-(dimethylaminomethylene)tetrahydrofuran-3-one

A solution of tetrahydrofuran-3-one (15.0 g, 174 mmol, 1 eq) in DMFDMA (40.36 g, 339 mmol, 45 mL, 1.94 eq) was stirred at 110° C. for 2 hr. The mixture was concentrated to afford 4-(dimethylaminomethylene)tetrahydrofuran-3-one (50 g, crude) as a red solid, which was used in the next step without further purification.

### Preparation of 4,6-dihydro-1H-furo[3,4-c]pyrazole

To a solution of 4-(dimethylaminomethylene)tetrahydrofuran-3-one (25 g, 177.09 mmol, 1 eq) in EtOH (150 mL) was added 98% NH₂NH₂·H₂O (9.05 g, 177 mmol, 8.78 mL, 1 eq) at 15° C. The solution was stirred at 80° C. for 20 hr under a N₂ atmosphere and then concentrated. The reaction was repeated once at the same scale. The residue of the two batches were combined and purified using silica gel chromatography (petroleum ether:ethyl acetate=1:1, 1:2) to afford 4,6-dihydro-1H-furo[3,4-c]pyrazole (2.2 g for two batches, 5.6% yield) as a yellow solid.

¹H NMR (400 MHz, CDCl₃) δ 7.19 (s, 1H), 4.85 (s, 2H), 4.82 (s, 2H).

### Preparation of 1-(tetrahydro-2H-pyran-2-yl)-4,6-dihydro-1H-furo[3,4-c]pyrazole

A solution of 4,6-dihydro-1H-furo[3,4-c]pyrazole (4.5 g, 40.9 mmol, 1 eq), DHP (4.47 g, 53.1 mmol, 4.86 mL, 1.3 eq) and TosOH (352 mg, 2.04 mmol, 0.05 eq) in toluene (50 mL) was stirred at 100° C. for 16 hr under a N₂ atmosphere. The mixture was concentrated, and the residue was purified using silica gel chromatography (petroleum ether:ethyl acetate=3:1, 1:1) to afford 1-(tetrahydro-2H-pyran-2-yl)-4,6-dihydro-1H-furo[3,4-c]pyrazole (3.8 g, 48% yield) as a colorless oil.

¹H NMR (300 MHz, CDCl₃) δ 7.31 (s, 1H), 5.39-5.28 (m, 1H), 4.89 (d, J=0.9 Hz, 2H), 4.85 (d, J=1.5 Hz, 2H), 4.13-4.03 (m, 1H), 3.77-3.64 (m, 1H), 2.28-2.07 (m, 2H), 1.71-1.62 (m, 4H).

### Preparation of 1-(tetrahydro-2H-pyran-2-yl)-4,6-dihydro-1H-furo[3,4-c]pyrazole-3-carbaldehyde

To a solution of 1-(tetrahydro-2H-pyran-2-yl)-4,6-dihydro-1H-furo[3,4-c]pyrazole (250 mg, 1.29 mmol, 1 eq) in THF (3 mL) was added n-BuLi (2.2 M in THF, 702 μL, 1.2 eq) at −70° C. under a N₂ atmosphere. Yellow suspensions were formed. After 5 min, DMF (188 mg, 2.57 mmol, 198 μL, 2 eq) in THF (1 mL) was added. The mixture was stirred at −70° C. for 10 min before being poured into water (20 mL) and stirred for 30 min. The quenched mixture was extracted with ethyl acetate (15 mL×2). The combined organic phase was washed with brine (15 mL×2), dried over anhydrous Na₂SO₄, and filtered. The filtrate was concentrated in vacuo. The residue was purified by Prep-TLC (petroleum ether:ethyl acetate=2:1) to afford 1-(tetrahydro-2H-pyran-2-yl)-4,6-dihydro-1H-furo[3,4-c]pyrazole-3-carbaldehyde (90 mg, 31% yield) as a white solid.

¹H NMR (400 MHz, CDCl₃) δ 9.97 (s, 1H), 5.98 (dd, J=2.4 Hz, 9.6 Hz, 1H), 5.13-5.02 (m, 2H), 4.93-4.83 (m, 2H), 4.10-4.03 (m, 1H), 3.79-3.70 (m, 1H), 2.42-2.31 (m, 1H), 2.16-2.05 (m, 2H), 1.79-1.59 (m, 3H).

### Preparation of 2-methoxy-N-((1-(tetrahydro-2H-pyran-2-yl)-4,6-dihydro-1H-furo[3,4-c]pyrazol-3-yl)methyl)pyridin-4-amine

A solution of 1-(tetrahydro-2H-pyran-2-yl)-4,6-dihydro-1H-furo[3,4-c]pyrazole-3-carbaldehyde (90 mg, 405 μmol, 1 eq), 2-methoxypyridin-4-amine (65 mg, 526 μmol, 1.3 eq) and TosOH (7 mg, 40 μmol, 0.1 eq) in toluene (10 mL) was stirred at 100° C. for 4 hr. The solution was cooled to 20° C. NaBH₃CN (76 mg, 1.21 mmol, 3 eq) was added to the solution, and the resulting mixture was stirred at 20° C. for 1 hr. The mixture was concentrated, and the residue was purified by reversed-phase HPLC (0.1% FA condition), followed by lyophilization to afford 2-methoxy-N-((1-(tetrahydro-2H-pyran-2-yl)-4,6-dihydro-1H-furo[3,4-c]pyrazol-3-yl) methyl)pyridin-4-amine (60 mg, 39% yield, FA salt) as a colorless gum.

### Preparation of 3-(3-chloro-4-fluorophenyl)-1-(2-methoxypyridin-4-yl)-1-((1-(tetrahydro-2H-pyran-2-yl)-4,6-dihydro-1H-furo[3,4-c]pyrazol-3-yl)methyl)urea

To a solution of 2-methoxy-N-((1-(tetrahydro-2H-pyran-2-yl)-4,6-dihydro-1H-furo[3,4-c]pyrazol-3-yl)methyl)pyridin-4-amine (60 mg, 159 μmol, 1 eq, FA salt) and DIPEA (62 mg, 478 μmol, 83 μL, 3 eq) in DCM (3 mL) was added 2-chloro-1-fluoro-4-isocyanatobenzene (41 mg, 239 μmol, 1.5 eq) at 0° C. The solution was stirred at 0° C. for 1 hr under a N₂ atmosphere. The solution was concentrated at 20° C. The crude product was purified by reversed-phase HPLC (0.1% NH₃—H₂O), followed by lyophilization to afford 3-(3-chloro-4-fluorophenyl)-1-(2-methoxypyridin-4-yl)-1-((1-(tetrahydro-2H-pyran-2-yl)-4,6-dihydro-1H-furo[3,4-c]pyrazol-3-yl)methyl)urea (20 mg, 25% yield) as a white solid.

### Preparation of 3-(3-chloro-4-fluorophenyl)-1-(4,6-dihydro-1H-furo[3,4-c]pyrazol-3-yl)methyl)-1-(2-methoxypyridin-4-yl))urea

To a solution of 3-(3-chloro-4-fluorophenyl)-1-(2-methoxypyridin-4-yl)-1-((1-(tetrahydro-2H-pyran-2-yl)-4,6-dihydro-1H-furo[3,4-c]pyrazol-3-yl)methyl)urea (20 mg, 40 μmol, 1 eq) in DCM (4 mL) was added TFA (1 mL) at 20° C. The solution was stirred at 20° C. for 1 hr. The mixture was concentrated, and the residue was purified by Prep-HPLC (column: Phenomenex Synergi C18 150 mm×25 mm×10 μm; mobile phase: [water (0.225% FA)-ACN]; B %: 27%-57%, 10 min), followed by lyophilization to afford 3-(3-chloro-4-fluorophenyl)-1-(4,6-dihydro-1H-furo[3,4-c]pyrazol-3-yl)methyl)-1-(2-methoxypyridin-4-yl))urea (3.3 mg, 18.6% yield, 94% purity) as a white solid.

[M+H⁺]=418.0,

¹H NMR (400 MHz, CD₃OD) δ 8.10 (d, J=5.6 Hz, 1H), 7.62 (dd, J=2.6 Hz, 6.8 Hz, 1H), 7.33-7.28 (m, 1H), 7.18-7.12 (m, 1H), 6.86 (dd, J=1.6 Hz, 5.6 Hz, 1H), 6.68 (d, J=1.2 Hz, 1H), 4.95 (s, 2H), 4.75 (s, 2H), 4.71 (s, 2H), 3.91 (s, 3H).

### Example 71. Preparation of 3-(3-chloro-4-fluorophenyl)-1-((5,6-dihydro-1H-[1,4]dioxino[2,3-c]pyrazol-3-yl)methyl)-1-(2-methoxypyridin-4-yl)urea

¹H NMR (400 MHz, Acetone) δ 8.11 (d, J=5.6 Hz, 1H), 7.83 (dd, J=6.7 Hz, 2.5 Hz, 1H), 7.43-7.36 (m, 1H), 7.19 (t, J=9.0 Hz, 1H), 6.96 (dd, J=5.6 Hz, 1.8 Hz, 1H), 6.77 (d, J=1.5 Hz, 1H), 4.87 (s, 2H), 4.26-4.20 (m, 2H), 4.14-4.07 (m, 2H), 3.88 (s, 3H).

### Example 72. Preparation of 3-(3-chloro-4-fluorophenyl)-1-((5-(difluoromethyl)-1H-pyrazol-3-yl)methyl)-1-(6-methoxypyridin-3-yl)urea

[M+H⁺]=426.1,

¹H NMR (400 MHz, DMSO-d₆) δ 13.14 (s, 1H), 8.28 (s, 1H), 8.06 (d, J=2.7 Hz, 1H), 7.74 (dd, J=6.9 Hz, 2.6 Hz, 1H), 7.59 (dd, J=8.8 Hz, 2.7 Hz, 1H), 7.43 (ddd, J=9.1 Hz, 4.3 Hz, 2.6 Hz, 1H), 7.28 (t, J=9.1 Hz, 1H), 7.11-6.71 (m, 2H), 6.36 (s, 1H), 4.86 (s, 2H), 3.87 (s, 3H).

### Example 73. Preparation of 3-(3-chloro-4-fluorophenyl)-1-(2-methoxypyridin-4-yl)-1-((1,4,6,7-tetrahydropyrano[4,3-c]pyrazol-3-yl)methyl)urea

[M+H⁺]=432.1,

¹H NMR (400 MHz, Methanol-d₄) δ 8.08 (d, J=5.7 Hz, 1H), 7.65 (dd, J=6.7 Hz, 2.6 Hz, 1H), 7.33 (ddd, J=9.0 Hz, 4.2 Hz, 2.6 Hz, 1H), 7.16 (t, J=9.0 Hz, 1H), 6.89 (dd, J=5.7 Hz, 2.0 Hz, 1H), 6.71 (d, J=1.9 Hz, 1H), 4.94 (s, 2H), 4.62 (s, 2H), 3.91 (s, 3H), 3.88 (t, J=5.6 Hz, 2H), 2.73 (t, J=5.6 Hz, 2H).

### Example 74. Preparation of 3-(3-chloro-4-fluorophenyl)-1-(2-methoxypyridin-4-yl)-1-((5-(trifluoromethyl)-1H-pyrazol-3-yl)methyl)urea

[M+H⁺]=445.3,

¹H NMR (300 MHz, CDCl₃) δ 8.27 (d, J=5.5 Hz, 1H), 7.47 (dd, J=6.4 Hz, 2.5 Hz, 1H), 7.10 (m, 2H), 6.73 (dd, J=5.5 Hz, 1.7 Hz, 1H), 6.61 (d, J=1.5 Hz, 1H), 6.43 (s, 1H), 6.37 (s, 1H), 4.81 (s, 2H), 3.99 (s, 3H).

### Example 75. Preparation of 3-(3-chloro-4-fluorophenyl)-1-(5-methoxypyridin-3-yl)-1-((1,4,6,7-tetrahydropyrano[4,3-c]pyrazol-3-yl)methyl)urea

[M+H⁺]=433.2,

¹H NMR (300 MHz, CDCl₃) δ 8.26 (d, J=2.7 Hz, 1H), 8.08 (d, J=2.0 Hz, 1H), 7.47 (dd, J=6.5 Hz, 2.6 Hz, 1H), 7.14-7.08 (m, 1H), 7.06-6.96 (m, 2H), 6.75 (s, 1H), 4.75 (s, 2H), 4.43 (s, 2H), 3.87 (t, J=5.6 Hz, 2H), 3.83 (s, 3H), 2.74 (t, J=5.6 Hz, 2H).

### Example 76. Preparation of 3-(3-chloro-4-fluorophenyl)-1-(5-methoxypyridin-3-yl)-1-((1,4,5,6-tetrahydropyrano[2,3-c]pyrazol-3-yl)methyl)urea

¹H NMR (400 MHz, DMSO-d₆) δ 11.56 (s, 1H), 8.42 (s, 1H), 8.23 (d, J=2.7 Hz, 1H), 8.02 (d, J=1.9 Hz, 1H), 7.73 (dd, J=6.9 Hz, 2.6 Hz, 1H), 7.43-7.39 (m, 1H), 7.31-7.27 (m, 2H), 4.70 (s, 2H), 4.05-4.02 (m, 2H), 3.81 (s, 3H), 2.28 (t, J=6.3 Hz, 2H), 1.80-1.70 (m, 2H).

### Example 77. Preparation of 3-(3-chloro-4-fluorophenyl)-1-((5-(difluoromethyl)-1H-pyrazol-3-yl)methyl)-1-(5-methoxypyridin-3-yl)urea

[M+H⁺]=427.9,

¹H NMR (400 MHz, CDCl₃) δ 11.27 (s, 1H), 8.39 (d, J=2.6 Hz, 1H), 8.15 (d, J=1.9 Hz, 1H), 7.55-7.39 (m, 1H), 7.07 (dd, J=8.9 Hz, 5.1 Hz, 2H), 6.93 (t, J=2.3 Hz, 1H), 6.70 (t, J=55.0 Hz, 1H), 6.31 (s, 1H), 6.18 (s, 1H), 4.78 (s, 2H), 3.86 (s, 3H).

### Example 78. Preparation of 3-(3-chloro-4-fluorophenyl)-1-(6-methoxypyridin-3-yl)-1-((4-methyl-5-(trifluoromethyl)-1H-pyrazol-3-yl)methyl)urea

[M+H⁺]=460.1,

¹H NMR (500 MHz, CDCl₃) δ 11.22 (s, 1H), 8.04 (d, J=2.7 Hz, 1H), 7.42 (dd, J=6.4 Hz, 2.6 Hz, 1H), 7.31 (dd, J=8.8 Hz, 2.7 Hz, 1H), 7.12-7.07 (m, 1H), 7.04 (t, J=8.7 Hz, 1H), 6.85 (d, J=8.8 Hz, 1H), 6.05 (s, 1H), 4.68 (s, 2H), 3.99 (s, 3H), 1.85 (s, 3H).

### Example 79. Preparation of 3-(3-chloro-4-fluorophenyl)-1-((1-(hydroxymethyl)-5-(trifluoromethyl)-1H-pyrazol-3-yl)methyl)-1-(6-methoxypyridin-3-yl)urea

[M+H⁺]=444.2,

¹H NMR (400 MHz, MeOD) δ 7.99-7.95 (m, 1H), 7.62-7.57 (m, 1H), 7.52-7.45 (m, 1H), 7.30-7.23 (m, 1H), 7.14-7.06 (m, 1H), 6.89-6.83 (m, 1H), 6.52-6.48 (m, 1H), 4.94-4.91 (m, 2H), 4.62-4.58 (m, 1H), 3.93 (s, 3H).

### Example 80. Preparation of 3-(3-chloro-4-fluorophenyl)-1-(2-methoxypyrimidin-5-yl)-1-((5-(trifluoromethyl)-1H-pyrazol-3-yl)methyl)urea

[M+H⁺]=445.03,

¹H NMR (400 MHz, Chloroform-d) δ 11.56 (s, 1H), 8.35 (s, 2H), 7.52 (dd, J=6.5 Hz, 2.7 Hz, 1H), 7.18 (ddd, J=8.9 Hz, 4.0 Hz, 2.7 Hz, 1H), 7.07 (t, J=8.7 Hz, 1H), 6.60 (s, 1H), 6.35 (s, 1H), 4.76 (s, 2H), 4.06 (s, 3H).

### Example 81. Preparation of 1-((4-bromo-5-(difluoromethyl)-1H-pyrazol-3-yl)methyl)-3-(3-chloro-4-fluorophenyl)-1-(2-methoxypyrimidin-5-yl)urea

[M+H⁺]=508.1,

¹H NMR (300 MHz, CDCl₃) δ 11.87 (s, 1H) 8.35 (s, 2H), 7.54 (dd, J=6.5 Hz, 2.6 Hz, 1H), 7.18 (ddd, J=8.9 Hz, 4.1 Hz, 2.7 Hz, 1H), 7.04 (dd, J=14.3 Hz, 5.5 Hz, 2H), 6.69 (t, J=53.5 Hz, 1H), 4.76 (s, 2H), 4.03 (s, 3H).

### Example 82. Preparation of 3-(3-chloro-4-fluorophenyl)-1-((5-(difluoromethyl)-4-(methoxymethyl)-1H-pyrazol-3-yl)methyl)-1-(6-methoxypyridin-3-yl)urea

[M+H⁺]=470.2,

¹H NMR (400 MHz, DMSO-d₆) δ 13.32-12.88 (m, 1H), 8.17 (s, 1H), 8.01 (d, J=2.6 Hz, 1H), 7.73 (dd, J=2.4 Hz, 6.9 Hz, 1H), 7.55 (dd, J=2.8H, 8.8 Hz, 1H), 7.43 (ddd, J=2.8 Hz, 4.3 Hz, 9.1 Hz, 1H), 7.27 (t, J=9.2 Hz, 1H), 7.12-6.74 (m, 2H), 4.90 (s, 2H), 4.24 (s, 2H), 3.85 (s, 3H), 3.09 (s, 3H).

### Example 83. Preparation of 3-(3-chloro-4-fluorophenyl)-1-((4-(2-ethoxyvinyl)-5-(trifluoromethyl)-1H-pyrazol-3-yl)methyl)-1-(6-methoxypyridin-3-yl)urea

[M+H⁺]=514.2,

¹H NMR (400 MHz, CDCl₃) δ 11.15 (br s, 1H), 7.98 (d, J=2.4 Hz, 1H), 7.36 (d, J=4.0 Hz), 7.27-7.25 (m, 1H), 7.09-6.97 (m, 2H), 6.78 (d, J=8.8 Hz, 1H), 6.06-5.99 (m, 2H), 5.23 (d, J=12.8 Hz, 1H), 4.65 (s, 3H), 3.91 (s, 3H), 3.59 (q, J=7.2 Hz, 2H), 1.15 (t, J=7.2 Hz, 3H).

### Example 84. Preparation of 3-(3-chloro-4-fluorophenyl)-1-((5-(difluoromethyl)-1H-pyrazol-3-yl)methyl)-1-(2-methoxypyrimidin-5-yl)urea

[M+H⁺]=427.0,

¹H NMR (300 MHz, CDCl₃) δ 8.40 (s, 2H), 7.51 (dd, J=6.5 Hz, 2.4 Hz, 1H), 7.16-7.03 (m, 2H), 6.72 (t, J=54.9 Hz, 1H), 6.34 (s, 1H), 6.07 (s, 1H), 4.76 (s, 2H), 4.11 (s, 3H).

### Example 85. Preparation of 3-(3-chloro-4-fluorophenyl)-1-((4-(2-ethoxyethyl)-5-(trifluoromethyl)-1H-pyrazol-3-yl)methyl)-1-(6-methoxypyridin-3-yl)urea

[M+H⁺]=516.2,

¹H NMR (400 MHz, CDCl₃) δ 8.03 (d, J=2.8 Hz, 1H), 7.43 (dd, J=2.5 Hz, 6.4 Hz, 1H), 7.32 (dd, J=2.8 Hz, 8.8 Hz, 1H), 7.14-7.08 (m, 1H), 7.07-7.02 (m, 1H), 6.84 (d, J=8.8 Hz, 1H), 6.04 (s, 1H), 4.76 (s, 2H), 3.99 (s, 3H), 3.31 (q, J=7.0 Hz, 2H), 3.26 (t, J=6.6 Hz, 2H), 2.55-2.44 (m, 2H), 1.10-1.07 (m, 3H).

### Example 86. Preparation of 3-(3-chloro-4-fluorophenyl)-1-((4-(2-hydroxyethyl)-5-(trifluoromethyl)-1H-pyrazol-3-yl)methyl)-1-(6-methoxypyridin-3-yl)urea

[M+H⁺]=487.9,

¹H NMR (400 MHz, CDCl₃) δ 8.06 (d, J=2.3 Hz, 1H), 7.42 (dd, J=2.6 Hz, 6.4 Hz, 1H), 7.38 (dd, J=2.8 Hz, 8.8 Hz, 1H), 7.11 (dd, J=2.7 Hz, 4.2 Hz, 1H), 7.09-7.03 (m, 1H), 6.87 (d, J=8.8 Hz, 1H), 6.09 (s, 1H), 4.81 (s, 2H), 4.00 (s, 3H), 3.61 (t, J=6.2 Hz, 2H), 2.55 (t, J=6.1 Hz, 2H).

### Example 87. Preparation of 3-(3-chloro-4-fluorophenyl)-1-(6-methoxypyridin-3-yl)-1-((4-((4,4,5,5-tetramethyl-1,3-dioxolan-2-yl)methyl)-5-(trifluoromethyl)-1H-pyrazol-3-yl)methyl)urea

¹H NMR (400 MHz, CDCl₃) δ 8.08 (d, J=2.6 Hz, 1H), 7.47-7.43 (m, 1H), 7.37-7.32 (m, 1H), 7.17-7.11 (m, 1H), 7.10-7.03 (m, 1H), 6.87 (d, J=8.8 Hz, 1H), 6.06 (s, 1H), 4.96-4.92 (m, 1H), 4.89-4.84 (m, 2H), 4.04-3.99 (m, 3H), 2.56 (d, J=4.5 Hz, 2H), 1.12 (s, 6H), 1.03-0.99 (m, 6H).

### Example 88. Preparation of 3-(3-chloro-4-fluorophenyl)-1-((5-(difluoromethyl)-4-(2-methoxyethyl)-1H-pyrazol-3-yl)methyl)-1-(6-methoxypyridin-3-yl)urea

¹H NMR (400 MHz, CDCl₃) δ 11.15 (s, 1H), 8.02 (d, J=2.6 Hz, 1H), 7.44 (dd, J=2.6 Hz, 6.4 Hz, 1H), 7.33 (dd, J=2.8 Hz, 8.8 Hz, 1H), 7.17-7.08 (m, 1H), 7.08-7.00 (m, 1H), 6.85 (s, 1H), 6.84-6.57 (m, 1H), 6.04 (s, 1H), 4.73 (s, 2H), 3.98 (s, 3H), 3.26 (t, J=6.6 Hz, 2H), 3.17 (s, 3H), 2.54 (t, J=6.5 Hz, 2H).

### Example 89. Preparation of 3-(4-chloro-5-fluoropyridin-2-yl)-1-(2-methoxypyrimidin-5-yl)-1-((5-(trifluoromethyl)-1H-pyrazol-3-yl)methyl)urea

[M+H⁺]=446.1,

¹H NMR (300 MHz, MeOD) δ 8.45 (s, 2H), 8.21 (d, J=5.6 Hz, 1H), 8.19 (d, J=1.0 Hz, 1H), 6.59 (s, 1H), 5.00 (s, 2H), 4.06 (s, 3H).

### Example 90. Preparation of 3-(3-chloro-4-fluorophenyl)-1-((5-methoxy-4-(trifluoromethyl)-1H-pyrazol-3-yl)methyl)-1-(6-methoxypyridin-3-yl)urea

### Preparation of 2-benzyl-5-(benzyloxymethyl)pyrazol-3-ol

To a solution of ethyl 4-benzyloxy-3-oxobutanoate (10.0 g, 42.3 mmol, 1 eq) in MeOH (80 mL) was added TsOH (729 mg, 4.23 mmol, 0.1 eq) and benzylhydrazine dihydrochloride salt (8.26 g, 42.3 mmol, 1 eq), and the resulting mixture was stirred at 80° C. for 16 hr. The reaction mixture was cooled to ambient temperature and concentrated to dryness under reduced pressure. The residue was dissolved in 2 N NaOH (25 mL) and the solution was extracted with EtOAc (1×30 mL). The aqueous phase was acidified with HCl until pH 3, and the precipitate was collected by filtration to afford 2-benzyl-5-(benzyloxymethyl)pyrazol-3-ol (3.98 g, 32%) as a yellow solid.

### Preparation of 1-benzyl-3-(benzyloxymethyl)-5-methoxy-1H-pyrazole

To a solution of methyl 4-methylbenzenesulfonate (1.39 g, 7.47 mmol, 1.1 eq) in DMF (10 mL) was added K₂CO₃ (2.35 g, 17.0 mmol, 2.5 eq) and 2-benzyl-5-(benzyloxymethyl)pyrazol-3-ol (2.0 g, 6.79 mmol, 1 eq). The mixture was stirred at 25° C. for 20 hr. The reaction mixture was filtered, and the filtrate was concentrated under reduced pressure. The crude product was purified by reversed-phase column chromatography (0.1% FA) to afford 1-benzyl-3-(benzyloxymethyl)-5-methoxy-1H-pyrazole (1.09 g, 52% yield) as a yellow oil.

### Preparation of 1-benzyl-3-(benzyloxymethyl)-4-iodo-5-methoxy-1H-pyrazole

To a mixture of 1-benzyl-3-(benzyloxymethyl)-5-methoxy-pyrazole (1.0 g, 3.24 mmol, 1 eq) in H₂O (5.5 mL) was added NaOAc (585 mg, 7.13 mmol, 2.20 eq), and the resulting mixture was heated to 100° C. before the addition of 2 (1.65 g, 6.48 mmol, 2 eq) and a solution consisting of KI (3.23 g, 19.4 mmol, 6 eq) and H₂O (7 mL). The reaction mixture was stirred at 100° C. for 10 min before being cooled to ambient temperature and partitioned between EtOAc (50 mL) and H₂O (20 mL). The organic phase was separated, washed with brine (30 mL), dried over anhydrous Na₂SO₄, filtered, and concentrated under reduced pressure. The residue was purified by column chromatography (petroleum ether:ethyl acetate=100:1 to 10:1) to afford 1-benzyl-3-(benzyloxymethyl)-4-iodo-5-methoxy-1H-pyrazole (1.14 g, 81% yield) as a white solid.

### Preparation of 1-benzyl-3-(benzyloxymethyl)-5-methoxy-4-(trifluoromethyl)-1H-pyrazole

To a solution of 1-benzyl-3-(benzyloxymethyl)-4-iodo-5-methoxy-pyrazole (533 mg, 1.23 mmol, 1 eq) in DMF (4 mL) was added CuI (584 mg, 3.07 mmol, 2.5 eq), KF (143 mg, 2.45 mmol, 2 eq), and trimethyl(trifluoromethyl)silane (872.27 mg, 6.13 mmol, 5 eq) under a N₂ atmosphere. The resulting mixture was stirred at 100° C. for 22 hr before being cooled to ambient temperature, filtered, and concentrated to dryness. The crude product was purified by reversed-phase HPLC (0.1% FA condition) to afford 1-benzyl-3-(benzyloxymethyl)-5-methoxy-4-(trifluoromethyl)-1H-pyrazole (180 mg, 39% yield) as a white solid.

### Preparation of (5-methoxy-4-(trifluoromethyl)-1H-pyrazol-3-yl)methanol

To a solution of 1-benzyl-3-(benzyloxymethyl)-5-methoxy-4-(trifluoromethyl)pyrazole (100 mg, 266 μmol, 1 eq) in MeOH (10 mL) was added 10% Pd/C (43 mg), and the resulting mixture was stirred at 50° C. for 16 hr under H₂ (15 psi). The mixture was filtered through a pad of Celite, and the filtrate was concentrated to dryness under reduced pressure to afford (5-methoxy-4-(trifluoromethyl)-1H-pyrazol-3-yl)methanol (67 mg, crude), which was used in the next step without further purification.

### Preparation of 3-(chloromethyl)-5-methoxy-4-(trifluoromethyl)-1H-pyrazole

A solution of [5-methoxy-4-(trifluoromethyl)-1H-pyrazol-3-yl]methanol (50 mg, 255 μmol, 1 eq) in SOCl₂ (2 mL) was stirred at 80° C. for 2 hr. The mixture was cooled to room temperature and concentrated to dryness under reduced pressure to afford 3-(chloromethyl)-5-methoxy-4-(trifluoromethyl)-1H-pyrazole (54 mg, crude) as a yellow oil.

### Preparation of 3-(chloromethyl)-5-methoxy-1-(tetrahydro-2H-pyran-2-yl)-4-(trifluoromethyl-1H-pyrazole

To a solution of 3-(chloromethyl)-5-methoxy-4-(trifluoromethyl)-1H-pyrazole (54 mg, 252 μmol, 1 eq) in DCM (5 mL) was added DHP (42 mg, 503 μmol, 46 μL, 2 eq), and the resulting mixture was stirred at 25° C. for 16 hr. The reaction mixture was concentrated to dryness under reduced pressure to afford 3-(chloromethyl)-5-methoxy-1-(tetrahydro-2H-pyran-2-yl)-4-(trifluoromethyl)-1H-pyrazole (70 mg, crude) as a yellow oil.

### Preparation of 6-methoxy-N-((5-methoxy-1-(tetrahydro-2H-pyran-2-yl)-4-(trifluoromethyl)-1H-pyrazol-3-yl)methyl)pyridine-3-amine

A mixture of 3-(chloromethyl)-5-methoxy-1-tetrahydropyran-2-yl-4-(trifluoromethyl)pyrazole (20 mg, 67 μmol, 1 eq), 6-methoxypyridin-3-amine (17 mg, 134 μmol, 2 eq) and K₂CO₃ (28 mg, 201 μmol, 3 eq) in CH₃CN (0.5 mL) was stirred at 70° C. for 1 hr. The reaction mixture was cooled to room temperature and concentrated. The residue was subjected to reversed-phase column chromatography (0.1% FA) to afford 6-methoxy-N-((5-methoxy-1-(tetrahydro-2H-pyran-2-yl)-4-(trifluoromethyl)-1H-pyrazol-3-yl)methy 1)pyridine-3-amine (30 mg, crude) as a colorless oil.

### Preparation of 3-(3-chloro-4-fluorophenyl)-1-((5-methoxy-1-(tetrahydro-2H-pyran-2-yl)-4-(trifluoromethyl)-1H-pyrazol-3-yl)methyl)-1-(6-methoxypyridin-3-yl)urea

A mixture of 6-methoxy-N-((5-methoxy-1-(tetrahydro-2H-pyran-2-yl)-4-(trifluoromethyl)-1H-pyrazol-3-yl)methyl)pyridine-3-amine (30 mg, 78 μmol, 1 eq), 2-chloro-1-fluoro-4-isocyanatobenzene (40 mg, 233 μmol, 3 eq) and DMAP (19 mg, 155 μmol, 2 eq) in CH₃CN (2 mL) was stirred at 60° C. for 30 min. The reaction mixture was cooled and concentrated. The residue was subjected to reversed-phase column chromatography (0.1% FA) to afford 3-(3-chloro-4-fluorophenyl)-1-((5-methoxy-1-(tetrahydro-2H-pyran-2-yl)-4-(trifluoro methyl)-1H-pyrazol-3-yl)methyl)-1-(6-methoxypyridin-3-yl)urea (20 mg, 46% yield) as a colorless oil.

### Preparation of 3-(3-chloro-4-fluorophenyl)-1-((5-methoxy-4-(trifluoromethyl)-1H-pyrazol-3-yl)methyl)-1-(6-methoxypyridin-3-yl)urea

A solution of 3-(3-chloro-4-fluorophenyl)-1-((5-methoxy-1-(tetrahydro-2H-pyran-2-yl)-4-(trifluoromethyl)-1H-pyrazol-3-yl)methyl)-1-(6-methoxypyridin-3-yl)urea (20 mg, 36 μmol, 1 eq) in TFA (1 mL) and DCM (2 mL) was stirred at 25° C. for 1 hr before being concentrated to dryness in vacuo. The residue was purified by Prep-HPLC (column: Phenomenex Gemini-NX C18 75 mm×30 mm×3 μm; mobile phase: [water (0.1% TFA)-ACN]; B %: 45%-75%, 7 min), followed by lyophilization to afford 3-(3-chloro-4-fluorophenyl)-1-((5-methoxy-4-(trifluoromethyl)-1H-pyrazol-3-yl)methy 1)-1-(6-methoxypyridin-3-yl)urea (5.5 mg, 32% yield, 99% purity) as a white solid.

[M+H⁺]=474.1,

¹H NMR (400 MHz, CDCl₃) δ 8.05 (d, J=2.4 Hz, 1H), 7.46 (dd, J=2.3 Hz, 6.4 Hz, 1H), 7.36 (dd, J=2.6 Hz, 8.7 Hz, 1H), 7.12-7.02 (m, 2H), 6.85 (d, J=8.7 Hz, 1H), 6.11 (s, 1H), 4.78 (s, 2H), 3.98 (d, J=8.3 Hz, 6H).

### Example 91. Preparation of 91. 3-(3-chloro-4-fluorophenyl)-1-((4-(2-hydroxyethyl)-5-(trifluoromethyl)-1H-pyrazol-3-yl)methyl)-1-(2-methoxypyrimidin-5-yl)urea

[M+H⁺]=489.1,

¹H NMR (400 MHz, CDCl₃) δ 8.37 (s, 1H), 7.46 (dd, J=2.8 Hz, 6.4 Hz 1H), 7.04-7.13 (m, 2H), 6.30 (s, 1H), 4.82 (s, 2H), 4.07 (s, 3H), 3.63 (t, J=6 Hz, 2H), 2.54 (t, J=6 Hz, 2H).

### Example 92. Preparation of 3-(3-chloro-4-fluorophenyl)-1-((5-(difluoromethyl)-4-(2-hydroxypropan-2-yl)-1H-pyrazol-3-yl)methyl)-1-(2-methoxypyrimidin-5-yl)urea

[M+H⁺]=485.1,

¹H NMR (400 MHz, CDCl₃) δ 8.48-8.40 (m, 2H), 7.54-7.49 (m, 1H), 7.18-7.13 (m, 1H), 7.11-7.03 (m, 1H), 6.99-6.68 (m, 1H), 6.37-6.25 (m, 1H), 5.24-5.09 (m, 2H), 3.99 (s, 3H), 1.52 (s, 6H).

### Example 93. Preparation of 3-(3-chloro-4-fluorophenyl)-1-((5-(difluoromethyl)-4-(prop-1-en-2-yl)-1H-pyrazol-3-yl)methyl)-1-(2-methoxypyrimidin-5-yl)urea

[M+H⁺]=467.1,

¹H NMR (400 MHz, CDCl₃) δ 11.61-10.76 (m, 1H), 8.39-8.32 (m, 2H), 7.52 (s, 1H), 7.16-7.11 (m, 1H), 7.10-7.05 (m, 1H), 6.84-6.54 (m, 1H), 6.24-6.12 (m, 1H), 5.12-5.04 (m, 1H), 4.80-4.71 (m, 2H), 4.60-4.50 (m, 1H), 4.10-4.02 (m, 3H), 1.86-1.81 (m, 3H).

### Example 94. Preparation of 3-(3-chloro-4-fluorophenyl)-1-(6-methoxypyridazin-4-yl)-1-((5-(trifluoromethyl)-1H-pyrazol-3-yl)methyl)urea

[M+H⁺]=445.4,

¹H NMR (500 MHz, Methanol-d₄) δ 8.03 (s, 1H), 7.71 (dd, J=6.7 Hz, 2.7 Hz, 1H), 7.37 (ddd, J=8.8 Hz, 4.2 Hz, 2.3 Hz, 1H), 7.21 (td, J=9.0 Hz, 1.7 Hz, 1H), 6.76 (s, 1H), 6.62 (s, 1H), 5.10 (s, 2H), 3.75 (s, 3H).

### Example 95. Preparation of 3-(3-chloro-4-fluorophenyl)-1-((4-(2-hydroxypropyl)-5-(trifluoromethyl)-1H-pyrazol-3-yl)methyl)-1-(2-methoxypyrimidin-5-yl)urea

[M+H⁺]=503.1,

¹H NMR (400 MHz, DMSO-d₆) δ 13.29 (s, 1H), 8.48 (s, 2H), 8.34 (s, 1H), 7.72 (dd, J=6.8 Hz, 2.4 Hz, 1H), 7.39-7.33 (m, 1H), 7.33-7.28 (m, 1H), 4.94 (s, 2H), 4.63 (s, 1H), 3.94 (s, 3H), 3.65-3.55 (m, 1H), 2.48-2.40 (m, 1H), 0.97 (d, J=6.0 Hz, 3H).

### Example 96. Preparation of 3-(3-chloro-4-fluorophenyl)-1-(6-methoxypyridazin-3-yl)-1-((5-(trifluoromethyl)-1H-pyrazol-3-yl)methyl)urea

[M+H⁺]=445. 1,

¹H NMR (400 MHz, DMSO-d₆) δ 13.92 (s, 1H), 9.40 (s, 1H), 8.52 (d, J=10.0 Hz, 1H), 7.98 (d, J=5.0 Hz, 1H), 7.48 (d, J=2.9 Hz, 1H), 7.31 (t, J=9.0 Hz, 1H), 7.26 (d, J=10.0 Hz, 1H), 6.81 (s, 1H), 5.45 (s, 2H), 3.83 (s, 3H).

### Example 97. Preparation of 3-(3-chloro-4-fluorophenyl)-1-((5-(difluoromethyl)-1H-pyrazol-3-yl)methyl)-1-(6-methoxypyridazin-3-yl)urea

[M+H⁺]=427.1,

¹H NMR (400 MHz, DMSO-d₆) δ 3.83 (s, 3H), 5.43 (s, 2H), 6.61 (s, 1H), 6.84 (s, 1H), 6.98 (s, 1H), 7.11 (s, 1H), 7.21-7.35 (m, 2H), 7.50 (br d, J=7.95 Hz, 1H), 7.98 (br d, J=5.50 Hz, 1H), 8.51 (s, 1H), 8.54 (s, 1H), 9.42 (br s, 1H), 13.38 (s, 1H).

### Example 98. Preparation of 1-((4-bromo-5-(difluoromethyl)-1H-pyrazol-3-yl)methyl)-3-(3-chloro-4-fluorophenyl)-1-(2-methoxypyrimidin-5-yl)urea

[M+H⁺]=507.0,

¹H NMR (400 MHz, DMSO-d₆) δ 13.73 (br s, 1H), 8.51-8.47 (m, 2H), 8.40 (s, 1H), 7.71 (dd, J=2.6 Hz, 6.8 Hz, 1H), 7.43-7.36 (m, 1H), 7.33-7.25 (m, 1H), 7.12-6.81 (m, 1H), 4.88 (s, 2H), 3.94 (s, 3H).

### Example 99. Preparation of 3-(3-chloro-4-fluorophenyl)-1-((5-(difluoromethyl)-1H-pyrazol-3-yl)methyl)-1-(6-methoxypyridazin-4-yl)urea

[M+H⁺]=427.3,

¹H NMR (500 MHz, Methanol-d₄) δ 8.05 (s, 1H), 7.71 (dd, J=6.7 Hz, 2.4 Hz, 1H), 7.44-7.33 (m, 1H), 7.21 (t, J=9.0 Hz, 1H), 6.94-6.63 (m, 2H), 6.53 (s, 1H), 5.08 (s, 2H), 3.74 (s, 3H).

### Example 100. Preparation of 3-(3-chloro-4-fluorophenyl)-1-((5-(difluoromethyl)-4-methoxy-1H-pyrazol-3-yl)methyl)-1-(6-methoxypyridin-3-yl)urea

¹H NMR (400 MHz, CDCl₃) δ 10.88 (s, 1H), 8.09 (d, J=2.2 Hz, 1H), 7.44 (dd, J=2.6 Hz, 6.5 Hz, 1H), 7.41 (dd, J=2.8 Hz, 8.7 Hz, 1H), 7.13-7.07 (m, 1H), 7.06-7.00 (m, 1H), 6.89-6.84 (m, 1H), 6.84-6.55 (m, 1H), 6.09 (s, 1H), 4.67 (s, 2H), 3.98 (s, 3H), 3.64 (s, 3H).

### Example 101. Preparation of 3-(3-chloro-4-fluorophenyl)-1-((4-(2-hydroxy-2-methylpropyl)-5-(trifluoromethyl)-1H-pyrazol-3-yl)methyl)-1-(2-methoxypyrimidin-5-yl)urea

[M+H⁺]=517.0,

¹H NMR (400 MHz, CDCl₃) δ 8.39-8.33 (m, 2H), 7.51-7.47 (m, 1H), 7.18-7.12 (m, 1H), 7.11-7.06 (m, 1H), 6.26-6.17 (m, 1H), 4.94 (s, 2H), 4.09 (s, 3H), 2.52-2.46 (m, 2H), 1.16 (s, 6H).

### Example 102. Preparation of 3-(3-chloro-4-fluorophenyl)-1-((4-(2-hydroxypropan-2-yl)-5-(trifluoromethyl)-1H-pyrazol-3-yl)methyl)-1-(2-methoxypyrimidin-5-yl)urea

[M+H⁺]=503.2,

¹H NMR (400 MHz, CDCl₃) δ 8.37 (s, 2H), 7.47 (dd, J=2.6 Hz, 6.4 Hz, 1H), 7.16-7.10 (m, 1H), 7.09-6.98 (m, 1H), 6.27 (s, 1H), 5.19 (s, 2H), 4.19-3.95 (m, 3H), 1.49 (s, 1H), 1.47 (s, 6H).

### Example 103. Preparation of 3-(3-chloro-4-fluorophenyl)-1-((5-(difluoromethyl)-4-(2-hydroxy-2-methylpropyl)-1H-pyrazol-3-yl)methyl)-1-(2-methoxypyrimidin-5-yl)urea

[M+H⁺]=499.0,

¹H NMR (400 MHz, CDCl₃) δ 8.37 (s, 2H), 7.52-7.48 (m, 1H), 7.18-7.12 (m, 1H), 7.11-7.05 (m, 1H), 6.92-6.63 (m, 1H), 6.21 (s, 1H), 4.96-4.90 (m, 2H), 4.11-4.04 (m, 3H), 2.56-2.50 (m, 2H), 1.31-1.26 (m, 1H), 1.17 (s, 6H).

### Example 104. Preparation of 3-(3-chloro-4-fluorophenyl)-1-((5-cyano-1H-pyrazol-3-yl)methyl)-1-(2-methoxypyrimidin-5-yl)urea

[M+H⁺]=402.2,

¹H NMR (400 MHz, DMSO-d₆) δ 13.9 (s, 1H), 8.55 (s, 2H), 8.44 (s, 1H), 7.82-7.62 (m, 1H), 7.49-7.35 (m, 1H), 7.30 (td, J=9.1 Hz, 1.4 Hz, 1H), 6.90 (s, 1H), 4.90 (s, 2H), 3.96 (s, 3H).

### Example 105. Preparation of 3-(3-chloro-4-fluorophenyl)-1-(3-methoxybicyclo[1.1.1]pentan-1-yl)-1-((5-(trifluoromethyl)-1H-pyrazol-3-yl)methyl)urea

[M+H⁺]=432.9,

¹H NMR (400 MHz, CDCl₃) δ 11.43-10.70 (m, 1H), 7.45 (dd, J=2.6 Hz, 6.4 Hz, 1H), 7.15-7.10 (m, 1H), 7.07-6.98 (m, 1H), 6.65-6.51 (m, 1H), 6.35 (s, 1H), 4.44-4.38 (m, 2H), 3.32-3.23 (m, 3H), 2.28-2.22 (m, 6H).

### Example 106. Preparation of 3-(6-chloro-5-fluoropyridin-2-yl)-1-(2-methoxypyrimidin-5-yl)-1-((5-(trifluoromethyl)-1H-pyrazol-3-yl)methyl)urea

[M+H⁺]=446.1,

¹H NMR (400 MHz, DMSO-d₆) δ 13.55 (br s, 1H), 9.52 (br s, 1H), 8.51 (s, 2H), 7.96-7.88 (m, 2H), 6.63 (s, 1H), 4.93 (s, 2H), 3.94 (s, 3H).

### Example 107. Preparation of 3-(5-chloro-6-fluoropyridin-3-yl)-1-(2-methoxypyrimidin-5-yl)-1-((5-(trifluoromethyl)-1H-pyrazol-3-yl)methyl)urea

[M+H⁺]=446.1,

¹H NMR (400 MHz, DMSO-d₆) δ 8.57 (s, 2H), 8.25 (dd, J=2.4 Hz, 8.4 Hz, 1H), 8.19 (t, J=2.0 Hz, 1H), 6.65 (s, 1H), 4.91 (s, 2H), 3.96 (s, 3H).

### Example 108. Preparation of 3-(3-chloro-4-fluorophenyl)-1-((1,3-dimethyl-1H-pyrazol-5-yl)methyl)-1-(4-methoxyphenyl)urea

[M+H⁺]=404.86,

¹H NMR (300 MHz, CDCl₃) δ 7.44 (dd, J=6.5 Hz, 2.6 Hz, 1H), 7.09 (ddd, J=8.9 Hz, 4.1 Hz, 2.7 Hz, 1H), 7.07-6.86 (m, 5H), 6.09 (s, 1H), 5.79 (s, 1H), 4.86 (s, 2H), 3.85 (s, 3H), 3.69 (s, 3H), 2.18 (s, 3H).

### Example 109. Preparation of 3-(3-chloro-4-fluorophenyl)-1-(3-(2-hydroxypropan-2-yl)isoxazol-5-yl)-1-((1,4,5,6-tetrahydropyrano[2,3-c]pyrazol-3-yl)methyl)urea

[M+H⁺]=450.2,

¹H NMR (300 MHz, MeOD) δ 7.65 (d, J=7.1 Hz, 1H), 7.30 (s, 1H), 7.14 (t, J=8.9 Hz, 1H), 5.11 (s, 2H), 4.74 (s, 1H), 3.91 (t, J=5.6 Hz, 2H), 3.63 (s, 2H), 2.76 (d, J=5.6 Hz, 2H), 1.29 (s, 6H).

### Example 110. Preparation of 3-(3-chloro-4-fluorophenyl)-1-(6-(2-hydroxypropan-2-yl)pyridin-3-yl)-1-((1,4,5,6-tetrahydropyrano[2,3-c]pyrazol-3-yl)methyl)urea

[M+H⁺]=460.2,

¹H NMR (400 MHz, CDCl₃) δ 8.43 (s, 1H), 7.56-7.48 (m, 2H), 7.44 (dd, J=2.6 Hz, 6.4 Hz, 1H), 7.17-7.09 (m, 1H), 7.07-7.01 (m, 1H), 6.17 (s, 1H), 4.66 (s, 2H), 4.21-4.14 (m, 2H), 2.13 (t, J=6.3 Hz, 2H), 1.87-1.77 (m, 2H), 1.59 (s, 6H).

### Example 111. Preparation of 3-(3-chloro-4-fluorophenyl)-1-(6-(2-hydroxypropan-2-yl)pyridin-3-yl)-1-((1,4,5,7-tetrahydropyrano[3,4-c]pyrazol-3-yl)methyl)urea

[M+H⁺]=460.3,

¹H NMR (400 MHz, DMSO) δ 12.45 (s, 1H), 8.52 (s, 1H), 8.31 (s, 1H), 7.72-7.70 (m, 1H), 7.65-7.60 (m, 2H), 7.38-7.32 (m, 1H), 7.29-7.27 (m, 1H), 5.24 (s, 1H), 4.84 (s, 2H) 4.60-4.56 (m, 2H), 3.68-3.64 (m, 2H), 2.33-2.29 (m, 2H), 1.44 (s, 6H).

### Example 112. Preparation of 3-(3-chloro-4-fluorophenyl)-1-(2-(2-hydroxypropan-2-yl)pyridin-4-yl)-1-((1,4,5,7-tetrahydropyrano[3,4-c]pyrazol-3-yl)methyl)urea

[M+H⁺]=460.2,

¹H NMR (400 MHz, CDCl₃) δ 8.60-8.54 (m, 1H), 7.52 (dd, J=2.6 Hz, 6.4 Hz, 1H), 7.34 (d, J=1.8 Hz, 1H), 7.18-7.13 (m, 1H), 7.12-7.09 (m, 1H), 7.09-7.02 (m, 2H), 4.86 (s, 2H), 4.71 (s, 2H), 3.82-3.77 (m, 2H), 2.35 (t, J=5.4 Hz, 2H), 1.55 (s, 6H).

### Example 113. Preparation of 3-(3-chloro-4-fluorophenyl)-1-(2-(2-hydroxypropan-2-yl)pyridin-4-yl)-1-((1,4,5,6-tetrahydropyrano[2,3-c]pyrazol-3-yl)methyl)urea

[M+H⁺]=460.3,

¹H NMR (400 MHz, MeOD) δ 8.44 (d, J=5.5 Hz, 1H), 7.64 (dd, J=2.6 Hz, 6.6 Hz, 1H), 7.57 (d, J=1.8 Hz, 1H), 7.31 (ddd, J=2.6 Hz, 4.2 Hz, 9.0 Hz, 1H), 7.17 (t, J=2.8 Hz, 1H), 7.16-7.11 (m, 1H), 4.94 (s, 2H), 4.16-4.08 (m, 2H), 2.39 (t, J=6.3 Hz, 2H), 1.89-1.78 (m, 2H), 1.52 (s, 6H).

### Example 114. Preparation of 3-(3-chloro-4-fluorophenyl)-1-(4-hydroxyphenyl)-1-((1,4,5,6-tetrahydrocyclopenta[c]pyrazol-3-yl)methyl)urea

[M+H⁺]=401.3,

¹H NMR (400 MHz, DMSO-d₆) δ 11.92 (s, 1H), 9.55 (s, 1H), 7.94 (s, 1H), 7.71 (dd, J=6.9 Hz, 2.6 Hz, 1H), 7.41 (td, J=4.5 Hz, 2.7 Hz, 1H), 7.24 (t, J=9.1 Hz, 1H), 6.97 (d, J=8.7 Hz, 2H), 6.74 (d, J=8.7 Hz, 2H), 4.67 (s, 2H), 2.51 (s, 2H), 2.33 (s, 4H).

### Example 115. Preparation of 3-(3-chloro-4-fluorophenyl)-1-(3-(2-hydroxypropan-2-yl)isoxazol-5-yl)-1-((1,4,5,7-tetrahydropyrano[3,4-c]pyrazol-3-yl)methyl)urea

[M+H⁺]=450.2,

¹H NMR (400 MHz, CDCl₃) δ 9.00 (s, 1H), 7.61 (dd, J=2.7 Hz, 6.5 Hz, 1H), 7.35-7.28 (m, 1H), 7.09 (t, J=8.7 Hz, 1H), 6.17 (s, 1H), 4.97 (s, 2H), 4.74 (s, 2H), 3.89 (t, J=5.6 Hz, 2H), 2.73 (t, J=5.5 Hz, 2H), 1.60 (s, 6H).

### Example 116. Preparation of 3-(3-chloro-4-fluorophenyl)-1-(3,4-dimethoxyphenyl)-1-((4,5,6,7-tetrahydro-1H-indazol-3-yl)methyl)urea

[M+H⁺]=460.1,

¹H NMR (300 MHz, CDCl₃) δ 7.46 (dd, J=6.5 Hz, 2.6 Hz, 1H), 7.18-7.06 (m, 1H), 7.00 (t, J=8.7 Hz, 1H), 6.89 (d, J=8.5 Hz, 1H), 6.77 (dd, J=8.4 Hz, 2.3 Hz, 1H), 6.60 (d, J=2.3 Hz, 1H), 6.33 (s, 1H), 4.71 (s, 2H), 3.92 (s, 3H), 3.80 (s, 3H), 3.49 (s, 1H), 2.65 (t, J=6.2 Hz, 2H), 2.22 (t, J=6.0 Hz, 2H), 1.84-1.57 (m, 4H).

### Example 117. Preparation of 3-(3-chloro-4-fluorophenyl)-1-(3,5-dimethoxyphenyl)-1-((4,5,6,7-tetrahydro-1H-indazol-3-yl)methyl)urea

[M+H⁺]=459.14,

¹H NMR (300 MHz, CDCl₃) δ 7.47 (dd, J=6.4 Hz, 2.6 Hz, 1H), 7.16-7.09 (m, 1H), 7.01 (t, J=8.7 Hz, 1H), 6.48 (d, J=8.0 Hz, 2H), 6.32 (d, J=2.1 Hz, 2H), 4.72 (s, 2H), 3.76 (s, 6H), 2.64 (t, J=6.1 Hz, 2H), 2.26 (t, J=6.0 Hz, 2H), 1.73 (ddt, J=15.7 Hz, 9.3 Hz, 4.8 Hz, 4H).

### Example 118. Preparation of 3-(3-chloro-4-fluorophenyl)-1-(6-cyano-5-methoxypyridin-3-yl)-1-((5-(trifluoromethyl)-1H-pyrazol-3-yl)methyl)urea

[M+H⁺]=467.24,

¹H NMR (400 MHz, DMSO-d₆) δ 13.54 (s, 1H), 8.84 (s, 1H), 8.31 (d, J=1.9 Hz, 1H), 7.89 (d, J=1.9 Hz, 1H), 7.75 (dd, J=6.9 Hz, 2.4 Hz, 1H), 7.46-7.20 (m, 2H), 6.63 (s, 1H), 5.07 (s, 2H), 3.96 (s, 3H).

### Example 119. Preparation of 3-(3-chloro-4-fluorophenyl)-1-(5-cyano-6-methoxypyridin-3-yl)-1-((5-(trifluoromethyl)-1H-pyrazol-3-yl)methyl)urea

[M+H⁺]=469.1,

¹H NMR (500 MHz, Chloroform-d) δ 11.43 (s, 1H), 8.22 (s, 1H), 7.79 (s, 1H), 7.47 (d, J=6.6 Hz, 1H), 7.19-7.05 (m, 2H), 6.37 (s, 1H), 6.06 (s, 1H), 4.76 (s, 2H), 4.15 (s, 3H).

### Example 120. Preparation of 3-(3-chloro-4-fluorophenyl)-1-(5-cyanopyridin-3-yl)-1-((1,4,5,6-tetrahydropyrano[2,3-c]pyrazol-3-yl)methyl)urea

[M+H⁺]=427.0,

¹H NMR (400 MHz, DMSO) δ 11.55 (s, 1H), 8.94 (s, 1H), 8.70 (s, 1H), 8.59 (s, 1H), 8.34-8.32 (m, 1H), 7.70-7.68 (m, 1H), 7.36-7.32 (m, 2H), 4.85 (s, 1H), 4.06-4.01 (m, 2H), 2.34-2.32 (m, 2H), 1.74 (s, 2H).

### Example 121. Preparation of 3-(3-chloro-4-fluorophenyl)-1-(6-cyanopyridin-3-yl)-1-((1,4,6,7-tetrahydropyrano[4,3-c]pyrazol-3-yl)methyl)urea

[M+H⁺]=427.1,

¹H NMR (400 MHz, DMSO-d₆) δ 11.55 (s, 1H), 8.94 (d, J=1.8 Hz, 1H), 8.70 (d, J=2.5 Hz, 1H), 8.59 (s, 1H), 8.33 (t, J=2.2 Hz, 1H), 7.69 (dd, J=6.8 Hz, 2.3 Hz, 1H), 7.44-7.25 (m, 2H), 4.84 (s, 2H), 4.17-3.94 (m, 2H), 2.32 (t, J=6.4 Hz, 2H), 1.87-1.66 (m, 2H).

### Example 122. Preparation of 3-(3-chloro-4-fluorophenyl)-1-(2-cyanopyridin-4-yl)-1-((1,4,5,6-tetrahydropyrano[2,3-c]pyrazol-3-yl)methyl)urea

[M+H⁺]=427.2,

¹H NMR (400 MHz, CDCl₃) δ 8.69 (br s, 1H), 8.61 (d, J=5.8 Hz, 1H), 7.69 (d, J=2.1 Hz, 1H), 7.57 (dd, J=2.8 Hz, 6.5 Hz, 1H), 7.52-7.45 (m, 1H), 7.34-7.27 (m, 1H), 7.09 (t, J=8.8 Hz, 1H), 4.81 (s, 2H), 4.28-4.21 (m, 2H), 2.33 (t, J=6.2 Hz, 2H), 1.94 (s, 2H).

### Example 123. Preparation of 3-(3-chloro-4-fluorophenyl)-1-(2-cyanopyridin-4-yl)-1-((1,4,6,7-tetrahydropyrano[4,3-c]pyrazol-3-yl)methyl)urea

[M+H⁺]=427.3,

¹H NMR (400 MHz, Methanol-d₄) δ 8.54 (d, J=5.7 Hz, 1H), 7.88 (d, J=2.3 Hz, 1H), 7.66 (dd, J=6.7 Hz, 2.7 Hz, 1H), 7.56 (dd, J=5.8 Hz, 2.3 Hz, 1H), 7.33 (ddd, J=9.0 Hz, 4.1 Hz, 2.7 Hz, 1H), 7.17 (t, J=8.9 Hz, 1H), 5.00 (s, 2H), 4.62 (s, 2H), 3.85 (t, J=5.6 Hz, 2H), 2.71 (t, J=5.6 Hz, 2H).

### Example 124. Preparation of 3-(3-chloro-4-fluorophenyl)-1-(6-cyanopyridin-3-yl)-1-((1,4,5,6-tetrahydropyrano[2,3-c]pyrazol-3-yl)methyl)urea

[M+H⁺]=427. 1,

¹H NMR (400 MHz, DMSO-d₆) δ 11.59 (s, 1H), 8.85 (s, 1H), 8.70 (s, 1H), 8.08 (d, J=8.2 Hz, 1H), 7.93 (d, J=8.6 Hz, 1H), 7.71 (dd, J=2.4 Hz, 6.8 Hz, 1H), 7.40-7.30 (m, 2H), 4.91 (s, 2H), 4.03 (s, 2H), 2.34-2.30 (m, 2H), 1.74 (s, 2H).

### Example 125. Preparation of 3-(3-chloro-4-fluorophenyl)-1-(6-cyanopyridin-3-yl)-1-((1,4,6,7-tetrahydropyrano[4,3-c]pyrazol-3-yl)methyl)urea

[M+H⁺]=427.2,

¹H NMR (500 MHz, Methanol-d₄) δ 8.63 (t, J=1.7 Hz, 1H), 7.90 (s, 2H), 7.62 (dd, J=6.7 Hz, 2.6 Hz, 1H), 7.30 (ddd, J=9.0 Hz, 4.2 Hz, 2.6 Hz, 1H), 7.17 (t, J=9.0 Hz, 1H), 4.97 (s, 2H), 4.66 (s, 2H), 3.89 (t, J=5.6 Hz, 2H), 2.74 (s, 2H).

### Example 126. Preparation of 3-(3-chloro-4-fluorophenyl)-1-(6-cyanopyridin-3-yl)-1-((5-(difluoromethyl)-1H-pyrazol-3-yl)methyl)urea

[M+H⁺]=421.2,

¹H NMR (500 MHz, Methanol-d₄) δ 8.68 (t, J=1.7 Hz, 1H), 7.97-7.85 (m, 2H), 7.65 (dd, J=6.7 Hz, 2.6 Hz, 1H), 7.31 (ddd, J=9.0 Hz, 4.1 Hz, 2.6 Hz, 1H), 7.17 (t, J=9.0 Hz, 1H), 6.71 (t, J=55.2 Hz, 1H), 6.46 (s, 1H), 5.09 (s, 2H).

### Example 127. Preparation of 3-(3-chloro-4-fluorophenyl)-1-(6-cyanopyridin-3-yl)-1-((1,4,5,7-tetrahydropyrano[3,4-c]pyrazol-3-yl)methyl)urea

[M+H⁺]=427.2,

¹H NMR (400 MHz, DMSO-d₆) δ 12.36 (s, 1H), 8.98 (s, 1H), 8.71 (d, J=2.0 Hz, 1H), 8.12-8.01 (m, 1H), 7.96 (d, J=8.1 Hz, 1H), 7.69 (dd, J=2.4 Hz, 6.8 Hz, 1H), 7.41-7.35 (m, 1H), 7.35-7.29 (m, 1H), 4.97 (s, 2H), 4.56 (br s, 2H), 3.70 (t, J=5.5 Hz, 2H), 2.43 (s, 2H).

### Example 128. Preparation of 3-(3-chloro-4-fluorophenyl)-1-(2-cyanopyridin-4-yl)-1-((1,4,5,7-tetrahydropyrano[3,4-c]pyrazol-3-yl)methyl)urea

### Preparation of 3-(azidomethyl)-1-(tetrahydro-2H-pyran-2-yl)-1,4,5,7-tetrahydropyrano[3,4-c]pyrazole

To a solution of (1-tetrahydropyran-2-yl-1,4,5,7-tetrahydropyrano[3,4-c]pyrazol-3-yl)methanol (300 mg, 1.26 mmol, 1 eq) in THF (5 mL) was added DPPA (520 mg, 1.89 mmol, 409 μL, 1.5 eq) and DBU (288 mg, 1.89 mmol, 285 μL, 1.5 eq) under a N₂ atmosphere. The reaction mixture was stirred at 20° C. for 48 hr before being quenched with H₂O (20 mL). The resulting mixture was extracted with EtOAc (10 mL×3). The combined organic layer was dried over anhydrous Na₂SO₄, filtered, and concentrated under reduced pressure. The residue was purified using flash silica gel chromatography (petroleum ether:ethyl acetate=100:1 to 1:1) to afford 3-(azidomethyl)-1-(tetrahydro-2H-pyran-2-yl)-1,4,5,7-tetrahydropyrano[3,4-c]pyrazole (200 mg, 56% yield, 93% purity) as a white oil.

### Preparation of (1-(tetrahydro-2H-pyran-2-yl)-1,4,5,7-tetrahydropyrano[3,4-c]pyrazol-3-yl)methanamine

To a solution of 3-(azidomethyl)-1-tetrahydropyran-2-yl-5,7-dihydro-4H-pyrano[3,4-c]pyrazole (200 mg, 760 μmol, 1 eq) in THF (4 mL) was added 10% Pd/C (800 mg). The resulting mixture was stirred at 20° C. for 1 hr under a H₂ atmosphere (15 psi). The reaction mixture was filtered through a pad of Celite, and the filtrate was concentrated to afford (1-(tetrahydro-2H-pyran-2-yl)-1,4,5,7-tetrahydropyrano[3,4-c]pyrazol-3-yl)methanamine (200 mg, 80% yield, 72% purity) as a colorless oil.

### Preparation of 4-((1-(tetrahydro-2H-pyran-2-yl)-1,4,5,7-tetrahydropyrano[3,4-c]pyrazol-3-yl)methylamino)picolinonitrile

To a solution of (1-(tetrahydro-2H-pyran-2-yl)-1,4,5,7-tetrahydropyrano[3,4-c]pyrazol-3-yl)methanamine (195 mg, 822 μmol, 1 eq) and 4-fluoropyridin-2-carbonitrile (100 mg, 822 μmol, 1 eq) in DMF (4 mL) was added K₂CO₃ (341 mg, 2.47 mmol, 3 eq). The resulting mixture was stirred at 100° C. for 16 hr. The mixture was poured into a saturated NH₄Cl solution (30 mL), followed by extraction with ethyl acetate (30 mL×3). The combined organic layer was dried over Na₂SO₄, filtered, and concentrated in vacuo. The crude product was purified by reversed-phase column chromatography (0.1% FA) to afford 4-((1-(tetrahydro-2H-pyran-2-yl)-1,4,5,7-tetrahydropyrano[3,4-c]pyrazol-3-yl)methylamino)picolinonitrile (80 mg, 27% yield, 95% purity) as a yellow solid.

### Preparation of 3-(3-chloro-4-fluorophenyl)-1-(2-cyanopyridin-4-yl)-1-((1-(tetrahydro-2H-pyran-2-yl)-1,4,5,7-tetrahydropyrano[3,4-c]pyrazol-3-yl)methyl)urea

To a solution of 4-((1-(tetrahydro-2H-pyran-2-yl)-1,4,5,7-tetrahydropyrano[3,4-c]pyrazol-3-yl)methylamino)picolinonitrile (60 mg, 177 μmol, 1 eq) and 2-chloro-1-fluoro-4-isocyanatobenzene (91 mg, 530 μmol, 3 eq) in CH₃CN (1.5 mL) was added DMAP (65 mg, 530 μmol, 3 eq). The mixture was stirred at 80° C. for 0.2 hr before being concentrated to dryness in vacuo. The residue was purified by reversed-phase chromatography (0.1% FA) to afford 3-(3-chloro-4-fluorophenyl)-1-(2-cyanopyridin-4-yl)-1-((1-(tetrahydro-2H-pyran-2-yl)-1,4,5,7-tetrahydropyrano[3,4-c]pyrazol-3-yl)methyl)urea (16 mg, 16% yield, 90% purity) as a white solid.

### Preparation of 3-(3-chloro-4-fluorophenyl)-1-(2-cyanopyridin-4-yl)-1-((1,4,5,7-tetrahydropyrano[3,4-c]pyrazol-3-yl)methyl)urea

To a solution of 3-(3-chloro-4-fluorophenyl)-1-(2-cyanopyridin-4-yl)-1-((1-(tetrahydro-2H-pyran-2-yl)-1,4,5,7-tetrahydropyrano[3,4-c]pyrazol-3-yl)methyl)urea (15 mg, 29 μmol, 1 eq) in DCM (2 mL) was added TFA (1 mL), and the resulting mixture was stirred at 20° C. for 2 hr. The mixture was concentrated, and the residue was purified by prep-HPLC (column: Phenomenex Synergi C18 150 mm×25 mm×10 μm; mobile phase: [water (0.225% FA)-ACN]; 30%-60%, 10 min) to afford 3-(3-chloro-4-fluorophenyl)-1-(2-cyanopyridin-4-yl)-1-((1,4,5,7-tetrahydropyrano[3,4-c]pyrazol-3-yl)methyl)urea (10.8 mg, 85% yield, 99% purity) as a white solid.

[M+H⁺]=427.2,

¹H NMR (400 MHz, DMSO-d₆) δ 12.40 (s, 1H), 9.50 (s, 1H), 8.59 (d, J=5.6 Hz, 1H), 8.09 (d, J=2.1 Hz, 1H), 7.75 (dd, J=2.3 Hz, 6.8 Hz, 1H), 7.64 (d, J=3.8 Hz, 1H), 7.42-7.40 (m, 2H), 5.03 (s, 2H), 4.58 (s, 2H), 3.72 (t, J=5.6 Hz, 2H), 2.49-2.46 (m, 2H).

### Example 129. Preparation of 3-(3-chloro-4-fluorophenyl)-1-(5-cyanopyridin-3-yl)-1-((1,4,6,7-tetrahydropyrano[4,3-c]pyrazol-3-yl)methyl)urea

[M+H⁺]=425.25,

¹H NMR (400 MHz, Chloroform-d) δ 10.52 (s, 1H), 8.92-8.61 (m, 2H), 7.97 (t, J=2.3 Hz, 1H), 7.84 (s, 1H), 7.50 (dd, J=6.5 Hz, 2.7 Hz, 1H), 7.21 (dt, J=8.9 Hz, 3.4 Hz, 1H), 7.07 (t, J=8.7 Hz, 1H), 4.82 (s, 2H), 4.45 (s, 2H), 3.91 (t, J=5.6 Hz, 2H), 2.77 (t, J=5.6 Hz, 2H).

### Example 130. Preparation of 3-(3-chloro-4-fluorophenyl)-1-(6-cyanopyridin-3-yl)-1-((5-(trifluoromethyl)-1H-pyrazol-3-yl)methyl)urea

[M+H⁺]=437.26,

¹H NMR (400 MHz, DMSO-d₆) δ 13.59 (s, 1H), 8.79 (d, J=2.4 Hz, 2H), 8.10 (d, J=8.4 Hz, 1H), 8.02 (dd, J=8.4 Hz, 2.6 Hz, 1H), 7.72 (dd, J=6.9 Hz, 2.4 Hz, 1H), 7.41-7.28 (m, 2H), 6.60 (s, 1H), 5.07 (s, 2H).

### Example 131. Preparation of 4-(3-(3-chloro-4-fluorophenyl)-1-((4,5,6,7-tetrahydro-1H-indazol-3-yl)methyl)ureido)benzoic acid

[M+H⁺]=443.3,

¹H NMR (500 MHz, DMSO-d₆) δ 8.73 (s, 1H), 7.92 (d, J=8.6 Hz, 2H), 7.71 (dd, J=6.8 Hz, 2.6 Hz, 1H), 7.47-7.37 (m, 3H), 7.30 (t, J=9.1 Hz, 1H), 4.90 (s, 2H), 2.55 (t, J=6.1 Hz, 2H), 2.29 (t, J=6.1 Hz, 2H), 1.71-1.58 (m, 4H).

### Example 132. Preparation of 3-(3-chloro-4-fluorophenyl)-1-(isoxazol-3-yl)-1-((4,5,6,7-tetrahydro-1H-indazol-3-yl)methyl)urea

[M+H⁺]=392.2,

¹H NMR (300 MHz, CDCl₃) δ 10.27 (s, 1H), 8.27 (d, J=1.7 Hz, 1H), 7.74 (dd, J=6.5 Hz, 2.6 Hz, 1H), 7.49-7.34 (m, 1H), 7.11 (t, J=8.8 Hz, 1H), 6.71 (d, J=1.6 Hz, 1H), 5.09 (s, 2H), 2.62 (t, J=5.6 Hz, 2H), 2.51 (t, J=5.6 Hz, 2H), 1.86-1.64 (m, 4H).

### Example 133. Preparation of 3-(3-chloro-4-fluorophenyl)-1-((4,5,6,7-tetrahydro-1H-indazol-3-yl)methyl)-1-(1,3,4-thiadiazol-2-yl)urea

[M+H⁺]=409.05,

¹H NMR (500 MHz, DMSO) δ 12.45 (s, 1H), 10.15 (s, 1H), 9.14 (s, 1H), 7.76 (d, J=4.9 Hz, 1H), 7.44 (dd, J=19.5 Hz, 10.5 Hz, 2H), 5.47 (s, 2H), 2.53 (s, 2H), 2.44 (s, 2H), 1.66 (d, J=5.4 Hz, 4H).

### Example 134. Preparation of 3-(3-chloro-4-fluorophenyl)-1-(isoxazol-3-yl)-1-((1,4,5,6-tetrahydrocyclopenta[c]pyrazol-3-yl)methyl)urea

[M+H⁺]=376.17,

¹H NMR (300 MHz, CDCl₃) δ 10.20 (s, 1H), 8.30 (d, J=1.8 Hz, 1H), 7.73 (dd, J=6.5 Hz, 2.6 Hz, 1H), 7.37 (ddd, J=8.9 Hz, 4.1 Hz, 2.7 Hz, 1H), 7.10 (t, J=8.8 Hz, 1H), 6.49 (d, J=1.7 Hz, 1H), 4.97 (s, 2H), 2.74-2.67 (m, 2H), 2.63-2.56 (m, 2H), 2.53-2.43 (m, 2H).

### Example 135. Preparation of 3-(3-chloro-4-fluorophenyl)-1-(1H-pyrazol-3-yl)-1-((1,4,5,6-tetrahydropyrano[2,3-c]pyrazol-3-yl)methyl)urea

[M+H⁺]=391.2,

¹H NMR (300 MHz, DMSO-d₆) δ 12.76 (s, 1H), 11.57 (s, 1H), 10.55 (s, 1H), 7.86 (dd, J=6.6 Hz, 2.5 Hz, 1H), 7.78 (d, J=2.5 Hz, 1H), 7.44-7.31 (m, 2H), 6.10 (d, J=2.4 Hz, 1H), 4.90 (s, 2H), 4.01 (t, J=4.65 Hz, 2H), 2.32 (t, J=6.4 Hz, 2H), 1.83-1.63 (m, 2H).

### Example 136. Preparation of 3-(3-chloro-4-fluorophenyl)-1-(isoxazol-3-yl)-1-((1,4,5,6-tetrahydropyrano[2,3-c]pyrazol-3-yl)methyl)urea

[M+H⁺]=392.3,

¹H NMR (400 MHz, CDCl₃) δ 10.18 (s, 1H), 8.32 (d, J=1.6 Hz, 1H), 7.72 (dd, J=2.6 Hz, 6.6 Hz, 1H), 7.40-7.34 (m, 1H), 7.11 (t, J=8.8 Hz, 1H), 6.44 (d, J=1.5 Hz, 1H), 4.90 (s, 2H), 4.26-4.19 (m, 2H), 2.62-2.56 (m, 2H), 2.00-1.92 (m, 2H).

### Example 137. Preparation of 1-(bicyclo[1.1.1]pentan-1-yl)-3-(3-chloro-4-fluorophenyl)-1-((5-(trifluoromethyl)-1H-pyrazol-3-yl)methyl)urea

[M+H⁺]=402.9,

¹H NMR (400 MHz, CDCl₃) δ 7.53 (dd, J=2.7 Hz, 6.4 Hz, 1H), 7.21 (ddd, J=2.7 Hz, 4.0 Hz, 8.9 Hz, 1H), 7.14-7.07 (m, 1H), 6.75 (s, 1H), 6.42 (s, 1H), 4.47 (s, 2H), 2.64 (s, 1H), 2.23 (s, 6H).

### Example 138. Preparation of 3-(6-chloro-4-(dimethylamino)-5-fluoropyridin-2-yl)-1-(2-methoxypyrimidin-5-yl)-1-((5-(trifluoromethyl)-1H-pyrazol-3-yl)methyl)urea

[M+H⁺]=489.2,

¹H NMR (400 MHz, CDCl₃) δ 11.69-10.42 (m, 1H), 8.33 (s, 2H), 7.41 (d, J=6.1 Hz, 1H), 6.57 (s, 1H), 6.34 (s, 1H), 4.72 (s, 2H), 4.09 (s, 3H), 3.14 (d, J=2.1 Hz, 6H).

### Example 139. Preparation of 3-(3-chloro-4-fluoro-5-methylphenyl)-1-((5-(difluoromethyl)-1H-pyrazol-3-yl)methyl)-1-(2-methoxypyrimidin-5-yl)urea

[M+H⁺]=441.1,

¹H NMR (400 MHz, CDCl₃) δ 8.37 (s, 2H), 7.32-7.27 (m, 1H), 7.01-6.96 (m, 1H), 6.87-6.51 (m, 1H), 6.31 (s, 1H), 5.96 (s, 1H), 4.73 (s, 2H), 4.08 (s, 3H), 2.27 (d, J=2.3 Hz, 3H).

### Example 140. Preparation of 3-(3-chloro-4-fluoro-5-methylphenyl)-1-(2-methoxypyrimidin-5-yl)-1-((5-(trifluoromethyl)-1H-pyrazol-3-yl)methyl)urea

[M+H⁺]=459.2,

¹H NMR (400 MHz, CDCl₃) δ 11.58-10.91 (m, 1H), 8.38 (s, 2H), 7.24 (d, J=2.8 Hz, 1H), 6.98 (dd, J=2.4 Hz, 5.8 Hz, 1H), 6.34 (s, 1H), 5.94 (s, 1H), 4.73 (s, 2H), 4.09 (s, 3H), 2.27 (d, J=2.3 Hz, 3H).

### Example 141. Preparation of 3-(6-chloro-3-fluoro-4-methylpyridin-2-yl)-1-(2-methoxypyrimidin-5-yl)-1-((5-(trifluoromethyl)-1H-pyrazol-3-yl)methyl)urea

[M+H⁺]=460.2,

¹H NMR (400 MHz, CDCl₃) δ 8.39 (s, 2H), 7.01 (d, J=3.8 Hz, 1H), 6.55 (s, 1H), 6.35 (s, 1H), 4.78 (s, 2H), 4.05 (s, 3H), 2.32 (d, J=1.3 Hz, 3H).

### Example 142. Preparation of 3-(6-chloro-5-fluoro-4-methylpyridin-2-yl)-1-(2-methoxypyrimidin-5-yl)-1-((5-(trifluoromethyl)-1H-pyrazol-3-yl)methyl)urea

[M+H⁺]=460.1,

¹H NMR (400 MHz, CDCl₃) δ 11.78-10.53 (m, 1H), 8.40-8.28 (m, 2H), 7.91 (d, J=4.5 Hz, 1H), 6.70 (s, 1H), 6.35 (s, 1H), 4.74 (s, 2H), 4.10 (s, 3H), 2.39 (d, J=1.5 Hz, 3H).

### Example 143. Preparation of 3-(2-chloropyrimidin-5-yl)-1-(2-methoxypyrimidin-5-yl)-1-((5-(trifluoromethyl)-1H-pyrazol-3-yl)methyl)urea

[M+H⁺]=429.1,

¹H NMR (400 MHz, CDCl₃) δ 8.81 (s, 2H), 8.32 (s, 2H), 7.28 (br s, 1H), 6.36 (s, 1H), 4.75 (s, 2H), 4.00 (s, 3H), 2.01 (s, 1H).

### Example 144. Preparation of 3-(3-cyanophenyl)-1-(2-methoxypyrimidin-5-yl)-1-((5-(trifluoromethyl)-1H-pyrazol-3-yl)methyl)urea

[M+H⁺]=418.2,

¹H NMR (400 MHz, CDCl₃) δ 11.51 (s, 1H), 8.35 (s, 2H), 7.75 (s, 1H), 7.60 (d, J=7.6 Hz, 1H), 7.41-7.37 (m, 2H), 6.73 (s, 1H), 6.36 (s, 1H), 4.77 (s, 2H), 4.05 (s, 3H).

### Example 145. Preparation of 3-(6-cyanopyridin-2-yl)-1-(2-methoxypyrimidin-5-yl)-1-((5-(trifluoromethyl)-1H-pyrazol-3-yl)methyl)urea

[M+H⁺]=419. 1,

¹H NMR (300 MHz, MeOD) δ 8.43 (s, 2H), 8.27 (d, J=8.6 Hz, 1H), 7.90 (dd, J=8.6 Hz, 7.5 Hz, 1H), 7.56-7.40 (m, 1H), 6.58 (s, 1H), 4.98 (s, 2H), 4.04 (s, 3H).

### Example 146. Preparation of 3-(2-cyanopyridin-4-yl)-1-(2-methoxypyrimidin-5-yl)-1-((5-(trifluoromethyl)-1H-pyrazol-3-yl)methyl)urea

[M+H⁺]=419.6,

¹H NMR (500 MHz, DMSO-d₆) δ 13.56 (s, 1H), 9.01 (s, 1H), 8.58 (s, 2H), 8.50 (d, J=5.7 Hz, 1H), 8.07 (s, 1H), 7.75 (d, J=5.6 Hz, 1H), 6.67 (s, 1H), 4.93 (s, 2H), 3.96 (s, 3H).

### Example 147. Preparation of 3-(3-cyanophenyl)-1-((5-(difluoromethyl)-1H-pyrazol-3-yl)methyl)-1-(2-methoxypyrimidin-5-yl)urea

[M+H⁺]=400. 1,

¹H NMR (400 MHz, CDCl₃) δ 11.77-10.63 (m, 1H), 8.36 (s, 2H), 7.77 (d, J=1.5 Hz, 1H), 7.55 (td, J=2.1 Hz, 7.5 Hz, 1H), 7.44-7.33 (m, 2H), 6.87-6.53 (m, 2H), 6.32 (s, 1H), 4.76 (s, 2H), 4.06 (s, 3H).

### Example 148. Preparation of 3-(6-cyanopyridin-2-yl)-1-(2-methoxypyrimidin-5-yl)-1-((5-(trifluoromethyl)-1H-pyrazol-3-yl)methyl)urea

[M+H⁺]=418.9,

¹H NMR (400 MHz, CDCl₃) δ 8.42-8.38 (m, 3H), 7.87 (t, J=7.9 Hz, 1H), 7.43 (d, J=7.6 Hz, 1H), 7.01 (s, 1H), 6.40 (s, 1H), 4.80 (s, 2H), 4.13 (s, 3H).

### Example 149. Preparation of 3-(3-cyano-4,5-difluorophenyl)-1-(2-methoxypyrimidin-5-yl)-1-((5-(trifluoromethyl)-1H-pyrazol-3-yl)methyl)urea

[M+H⁺]=454.1,

¹H NMR (400 MHz, DMSO-d₆) δ 8.52 (s, 2H), 7.80 (q, J=9.5 Hz, 1H), 7.26 (br dd, J=2.8 Hz, 9.0 Hz, 1H), 6.69 (s, 1H), 4.91 (s, 2H), 3.95 (s, 3H).

### Example 150. Preparation of 3-(3-cyano-4,5-difluorophenyl)-1-((5-(difluoromethyl)-1H-pyrazol-3-yl)methyl)-1-(2-methoxypyrimidin-5-yl)urea

[M+H⁺]=436.1,

¹H NMR (400 MHz, CDCl₃) δ 8.42 (s, 2H), 7.84 (ddd, J=1.9 Hz, 3.8 Hz, 9.3 Hz, 1H), 7.43 (q, J=9.4 Hz, 1H), 6.87-6.55 (m, 2H), 6.36 (s, 1H), 4.78 (s, 2H), 4.08 (s, 3H).

### Example 151. Preparation of 3-(6-cyano-4,5-difluoropyridin-2-yl)-1-(2-methoxypyrimidin-5-yl)-1-((5-(trifluoromethyl)-1H-pyrazol-3-yl)methyl)urea

[M+H⁺]=455.1,

¹H NMR (400 MHz, CDCl₃) δ 11.33-10.86 (m, 1H), 8.41-8.36 (m, 3H), 7.01-6.93 (m, 1H), 6.43-6.37 (m, 1H), 4.78 (s, 2H), 4.13 (s, 3H).

### Example 152. Preparation of 3-(4-cyano-3-(difluoromethyl)phenyl)-1-(2-methoxypyrimidin-5-yl)-1-((5-(trifluoromethyl)-1H-pyrazol-3-yl)methyl)urea

[M+H⁺]=468.05,

¹H NMR (400 MHz, DMSO-d₆) δ 13.57 (s, 1H) δ 8.87 (s, 1H), 8.58 (s, 2H), 7.94 (s, 1H), 7.87 (q, J=8.7 Hz, 2H), 7.20 (t, J=54.3 Hz, 1H), 6.64 (s, 1H), 4.93 (s, 2H), 3.95 (s, 3H).

### Example 153. Preparation of 1-((5-cyano-1H-pyrazol-3-yl)methyl)-3-(4-cyano-3-(difluoromethyl)phenyl)-1-(2-methoxypyrimidin-5-yl)urea

[M+H⁺]=425.2,

¹H NMR (500 MHz, DMSO-d₆) δ 13.89 (s, 1H), 8.91 (s, 1H), 8.59 (s, 2H), 7.94 (s, 1H), 7.90 (d, J=8.6 Hz, 1H), 7.84 (d, J=8.7 Hz, 1H), 7.21 (t, J=54.4 Hz, 1H), 6.91 (s, 1H), 4.93 (s, 2H), 3.96 (s, 3H).

### Example 154. Preparation of 3-(3-cyano-4-fluorophenyl)-1-(2-methoxypyrimidin-5-yl)-1-((5-(trifluoromethyl)-1H-pyrazol-3-yl)methyl)urea

[M+H⁺]=436.3,

¹H NMR (500 MHz, Methanol-d₄) δ 8.45 (s, 2H), 7.87 (dd, J=6.0 Hz, 2.7 Hz, 1H), 7.69 (ddd, J=9.0 Hz, 4.7 Hz, 2.6 Hz, 1H), 7.27 (t, J=9.0 Hz, 1H), 6.58 (s, 1H), 4.98 (s, 2H), 4.07 (s, 3H).

### Example 155. Preparation of 3-(3-cyano-4-fluorophenyl)-1-((5-(difluoromethyl)-1H-pyrazol-3-yl)methyl)-1-(2-methoxypyrimidin-5-yl)urea

[M+H⁺]=418. 1,

¹H NMR (300 MHz, DMSO) δ 13.19 (s, 1H), 8.58 (s, 1H), 8.52 (s, 2H), 7.94 (dd, J=5.8 Hz, 2.7 Hz, 1H), 7.77 (ddd, J=9.2, 4.9 Hz, 2.8 Hz, 1H), 7.44 (t, J=9.1 Hz, 1H), 6.93 (t, J=54.5 Hz, 1H), 6.45 (s, 1H), 4.90 (s, 2H), 3.95 (s, 3H).

### Example 156. Preparation of 3-(4-cyano-3-fluorophenyl)-1-(2-methoxypyrimidin-5-yl)-1-((5-(trifluoromethyl)-1H-pyrazol-3-yl)methyl)urea

[M+H⁺]=436.1,

¹H NMR (400 MHz, DMSO) δ 8.56 (s, 1H), 7.81-7.66 (m, 1H), 7.41 (d, J=9.3 Hz, 1H), 6.64 (s, 1H), 4.92 (s, 1H), 3.95 (s, 1H).

### Example 157. Preparation of 1-((5-cyano-1H-pyrazol-3-yl)methyl)-3-(3-cyano-4-fluorophenyl)-1-(2-methoxypyrimidin-5-yl)urea

[M+H⁺]=393.2,

¹H NMR (500 MHz, DMSO-d₆) δ 13.87 (s, 1H), 8.59 (s, 1H), 8.55 (s, 2H), 7.93 (dd, J=6.2 Hz, 2.8 Hz, 1H), 7.75 (dt, J=8.2 Hz, 3.5 Hz, 1H), 7.44 (t, J=9.1 Hz, 1H), 6.91 (s, 1H), 4.90 (s, 2H), 3.96 (s, 3H).

### Example 158. Preparation of 3-(2-cyano-3-fluoropyridin-4-yl)-1-(2-methoxypyrimidin-5-yl)-1-((5-(trifluoromethyl)-1H-pyrazol-3-yl)methyl)urea

[M+H⁺]=437.1,

¹H NMR (500 MHz, DMSO-d₆) δ 13.57 (s, 1H), 9.02 (s, 1H), 8.58 (s, 2H), 8.46 (d, J=5.4 Hz, 1H), 8.05 (t, J=5.9 Hz, 1H), 6.65 (s, 1H), 4.95 (s, 2H), 3.95 (s, 3H).

### Example 159. Preparation of 3-(3-chloro-4-fluorophenyl)-1-((1,3-dimethyl-1H-pyrazol-4-yl)methyl)-1-(4-methoxyphenyl)urea

¹H NMR (300 MHz, DMSO) δ 7.90 (br, 1H), 7.73 (dd, J=6.90 Hz, 2.61 Hz, 1H), 7.40 (m, 2H), 7.27 (t, J=9.15 Hz, 1H), 7.07 (d, J=8.91 Hz, 2H), 6.96 (d, J=8.94 Hz, 2H), 4.56 (s, 2H), 3.76 (s, 3H), 3.66 (s, 3H), 1.80 (s, 3H).

### Example 160. Preparation of 3-(6-cyano-5-fluoropyridin-2-yl)-1-(2-methoxypyrimidin-5-yl)-1-((5-(trifluoromethyl)-1H-pyrazol-3-yl)methyl)urea

¹H NMR (300 MHz, DMSO-d₆) δ 13.59 (s, 1H), 9.50 (s, 1H), 8.49 (s, 2H), 8.12-7.83 (m, 2H), 6.62 (s, 1H), 4.92 (s, 2H), 3.93 (s, 3H).

### Example 161. Preparation of 3-(4-cyano-5-fluoropyridin-2-yl)-1-(2-methoxypyrimidin-5-yl)-1-((5-(trifluoromethyl)-1H-pyrazol-3-yl)methyl)urea

[M+H⁺]=437.2,

¹H NMR (400 MHz, CDCl₃) δ 8.44 (d, J=4.5 Hz, 1H), 8.41-8.35 (m, 2H), 8.22 (s, 1H), 6.94 (s, 1H), 6.38 (s, 1H), 4.78 (s, 2H), 4.14-4.06 (m, 3H).

### Example 162. Preparation of 3-(3-cyano-4-fluorophenyl)-1-(3-methoxybicyclo[1.1.1]pentan-1-yl)-1-((5-(trifluoromethyl)-1H-pyrazol-3-yl)methyl)urea

[M+H⁺]=424.2,

¹H NMR (400 MHz, CDCl₃) δ 7.74 (dd, J=2.8 Hz, 5.3 Hz, 1H), 7.61 (ddd, J=2.8 Hz, 4.4 Hz, 9.0 Hz, 1H), 7.21 (t, J=8.6 Hz, 1H), 6.81 (s, 1H), 6.45 (s, 1H), 4.52 (s, 2H), 3.37 (s, 3H), 2.36 (s, 6H).

### Example 163. Preparation of 3-(3-cyano-4-fluorophenyl)-1-(6-methoxypyridazin-4-yl)-1-((5-(trifluoromethyl)-1H-pyrazol-3-yl)methyl)urea

### Preparation of phenyl 3-cyano-4-fluorophenylcarbamate

To a mixture of 5-amino-2-fluorobenzonitrile (1 g, 7.35 mmol, 1 eq) in DCM (20 mL) was added Py (697 mg, 8.82 mmol, 712 μL, 1.2 eq) and phenyl carbonochloridate (1.27 g, 8.08 mmol, 1.01 mL, 1.1 eq). The mixture was stirred at 25° C. for 1 hr. The mixture was poured into citric acid aqueous solution (5%, 20 mL), and the resulting mixture was extracted with DCM (20 mL×3). The combined organic layer was dried over Na₂SO₄, filtered and concentrated in vacuo to afford phenyl 3-cyano-4-fluorophenylcarbamate (1.7 g, 6.63 mmol, 90.31% yield) as a colorless oil. The residue was used in the next step without further purification.

### Preparation of 3-(3-cyano-4-fluorophenyl)-1-(6-methoxypyridazin-4-yl)-1-((1-tetrahydro-2H-pyran-2-yl-5-(trifluoromethyl)-1H-pyrazol-3-yl)methyl)urea

To a mixture of 6-methoxy-N-((1-tetrahydro-2H-pyran-2-yl-5-(trifluoromethyl)-1H-pyrazol-3-yl) methyl)pyridazin-4-amine (25 mg, 70 μmol, 1 eq) in DMF (1 mL) was added NaH (4 mg, 84 μmol, 60% purity, 1.2 eq). The mixture was stirred at 30° C. for 30 min. Then phenyl N-(3-cyano-4-fluorophenyl)carbamate (27 mg, 104.94 μmol, 1.5 eq) was added to the mixture, the resulting mixture was stirred at 30° C. for 5 min. The mixture was poured into a saturated NH₄Cl solution (10 mL), and the resulting mixture was extracted with EtOAc (10 mL×3). The combined organic layer was dried over Na₂SO₄, filtered and concentrated in vacuo. The residue was purified by prep-TLC (petroleum ether:ethyl acetate=1:1), followed by filtration and concentration to afford 3-(3-cyano-4-fluorophenyl)-1-(6-methoxypyridazin-4-yl)-1-((1-tetrahydro-2H-pyran-2-yl-5-(trifluoromethyl)-1H-pyrazol-3-yl)methyl)urea (15 mg, 29 μmol, 41.27% yield) as a yellow oil.

### Preparation of 3-(3-cyano-4-fluorophenyl)-1-(6-methoxypyridazin-4-yl)-1-((5-(trifluoromethyl)-1H-pyrazol-3-yl)methyl)urea

To a mixture of 3-(3-cyano-4-fluorophenyl)-1-(6-methoxypyridazin-4-yl)-1-((1-tetrahydro-2H-pyran-2-yl-5-(trifluoromethyl)-1H-pyrazol-3-yl)methyl)urea (15 mg, 29 μmol, 1 eq) in DCM (3 mL) was added TFA (1.54 g, 13.51 mmol, 1 mL, 467.72 eq). The mixture was stirred at 30° C. for 1 hr and concentrated. The residue was purified by prep-HPLC (column: Unisil 3-100 C18 Ultra 150 mm×50 mm×3 um; mobile phase: [water (0.225% FA)-ACN]; B %: 35%-55%, 10 min), followed by lyophilization to afford 3-(3-cyano-4-fluorophenyl)-1-(6-methoxypyridazin-4-yl)-1-((5-(trifluoromethyl)-1H-pyrazol-3-yl)methyl)urea (6.3 mg, 14.33 μmol, 49.61% yield, 99% purity) as an off-white solid.

[M+H⁺]=436.1,

¹H NMR (400 MHz, CDCl₃) δ 8.79-8.69 (m, 1H), 7.85-7.76 (m, 1H), 7.75-7.67 (m, 1H), 7.58-7.42 (m, 1H), 7.21-7.16 (m, 1H), 6.90-6.73 (m, 1H), 6.37 (s, 1H), 4.98-4.79 (m, 2H), 4.14-4.00 (m, 3H).

### Example 164. Preparation of 3-(6-cyano-4-(dimethylamino)-5-fluoropyridin-2-yl)-1-(2-methoxypyrimidin-5-yl)-1-((5-(trifluoromethyl)-1H-pyrazol-3-yl)methyl)urea

[M+H⁺]=480.2,

¹H NMR (400 MHz, CDCl₃) δ 11.69-10.79 (m, 1H), 8.36 (s, 2H), 7.64 (d, J=7.0 Hz, 1H), 6.75 (s, 1H), 6.37 (s, 1H), 4.74 (s, 2H), 4.10 (s, 3H), 3.18 (d, J=2.1 Hz, 6H).

### Example 165. Preparation of 3-(2,6-dichloropyridin-3-yl)-1-(2-methoxypyrimidin-5-yl)-1-((5-(trifluoromethyl)-1H-pyrazol-3-yl)methyl)urea

[M+H⁺]=462. 1,

¹H NMR (400 MHz, CDCl₃) δ 11.14 (br s, 1H), 8.54 (d, J=8.4 Hz, 1H), 8.44 (s, 2H), 7.31 (d, J=8.6 Hz, 1H), 6.62 (s, 1H), 6.39 (s, 1H), 4.77 (s, 2H), 4.11 (s, 3H).

### Example 166. Preparation of 3-(2,6-dichloropyridin-3-yl)-1-((5-(difluoromethyl)-1H-pyrazol-3-yl)methyl)-1-(2-methoxypyrimidin-5-yl)urea

[M+H⁺]=444.1,

¹H NMR (400 MHz, CDCl₃) δ 8.56 (d, J=8.6 Hz, 1H), 8.44 (s, 2H), 7.31 (d, J=8.6 Hz, 1H), 6.86-6.54 (m, 2H), 6.36 (s, 1H), 4.77 (s, 2H), 4.10 (s, 3H).

### Example 167. Preparation of 3-(3,4-dicyanophenyl)-1-(2-methoxypyrimidin-5-yl)-1-((5-(trifluoromethyl)-1H-pyrazol-3-yl)methyl)urea

[M+H⁺]=443.1,

¹H NMR (500 MHz, Methanol-d₄) δ 8.45 (s, 2H), 8.18 (d, J=2.2 Hz, 1H), 7.88 (dd, J=8.8 Hz, 2.2 Hz, 1H), 7.82 (d, J=8.7 Hz, 1H), 6.60 (s, 1H), 5.00 (s, 2H), 4.07 (s, 3H).

### Example 168. Preparation of 3-(3,4-difluorophenyl)-1-(6-methoxypyridin-3-yl)-1-((5-(trifluoromethyl)-1H-pyrazol-3-yl)methyl)urea

[M+H⁺]=428.2,

¹H NMR (500 MHz, CDCl₃) δ 11.40 (s, 1H), 8.06 (d, J=2.7 Hz, 1H), 7.43-7.30 (m, 2H), 7.05 (dd, J=18.6 Hz, 8.9 Hz, 1H), 6.92-6.78 (m, 2H), 6.33 (s, 1H), 6.09 (s, 1H), 4.74 (s, 2H), 4.00 (s, 3H).

### Example 169. Preparation of 3-(3,6-difluoropyridin-2-yl)-1-(2-methoxypyrimidin-5-yl)-1-((5-(trifluoromethyl)-1H-pyrazol-3-yl)methyl)urea

[M+H⁺]=430.2,

¹H NMR (400 MHz, CDCl₃) δ 11.47-10.54 (m, 1H), 8.41 (s, 2H), 7.67-7.47 (m, 1H), 6.80-6.71 (m, 1H), 6.36 (s, 1H), 6.32 (s, 1H), 4.78 (s, 2H), 4.07 (s, 3H).

### Example 170. Preparation of 3-(5,6-difluoropyridin-2-yl)-1-(2-methoxypyrimidin-5-yl)-1-((5-(trifluoromethyl)-1H-pyrazol-3-yl)methyl)urea

### Preparation of 3-(5,6-difluoropyridin-2-yl)-1-(2-methoxypyrimidin-5-yl)-1-((1-(tetrahydro-2H-pyran-2-yl)-5-(trifluoromethyl-1H-pyrazol-3-yl)methyl)urea

To a mixture of 5,6-difluoropyridin-2-amine (55 mg, 423 μmol, 1 eq) and DIPEA (164 mg, 1.27 mmol, 221 μL, 3 eq) in DCM (2 mL) was added CDI (89 mg, 550 μmol, 1.3 eq), and the resulting solution was stirred at 30° C. for 16 hr. Then 2-methoxy-N-((1-(tetrahydro-2H-pyran-2-yl)-5-(trifluoromethyl)-1H-pyrazol-3-yl)methyl)pyrimidin-5-amine (100 mg, 280 μmol, 0.66 eq) was added, and the solution was stirred at 30° C. for 20 hr before being concentrated to afford 3-(5,6-difluoropyridin-2-yl)-1-(2-methoxypyrimidin-5-yl)-1-((1-(tetrahydro-2H-pyran-2-yl)-5-(trifluoromethyl)-1H-pyrazol-3-yl)methyl)urea (0.22 g, crude) as a gray solid. The crude product obtained was used in the next step without further purification.

### Preparation of 3-(5,6-difluoropyridin-2-yl)-1-(2-methoxypyrimidin-5-yl)-1-((5-(trifluoromethyl)-1H-pyrazole-3-yl)methyl)urea

A solution of 3-(5,6-difluoropyridin-2-yl)-1-(2-methoxypyrimidin-5-yl)-1-((1-(tetrahydro-2H-pyran-2-yl)-5-(trifluoromethyl)-1H-pyrazol-3-yl)methyl)urea obtained in the previous step (0.22 g, 429 μmol, 1 eq) in 4 M HCl (1 mL) and CH₃CN (1 mL) was stirred at 30° C. for 2 hr and at 40° C. for 1 hr. The reaction mixture was poured into a saturated NaHCO₃ aqueous solution (30 mL), and the resulting mixture was extracted with ethyl acetate (15 mL×3). The combined organic layer was washed with brine (10 mL), dried over anhydrous Na₂SO₄, and filtered. The filtrate was concentrated in vacuo. The crude product was purified by Prep-HPLC (column: Shim-pack C18 150 mm×25 mm×10 μm; mobile phase: [water (0.225% FA)-ACN]; B %: 42%-62%, 10 min), followed by lyophilization to afford 3-(5,6-difluoropyridin-2-yl)-1-(2-methoxypyrimidin-5-yl)-1-((5-(trifluoromethyl)-1H-pyrazole-3-yl)methyl)urea (12.3 mg, 6.6% yield, 99% purity) as a white solid.

[M+H⁺]=430.0,

¹H NMR (400 MHz, CDCl₃) δ 11.46-11.15 (m, 1H), 8.37 (s, 2H), 7.96 (dd, J=2.6 Hz, 8.7 Hz, 1H), 7.61 (q, J=8.7 Hz, 1H), 6.68 (s, 1H), 6.37 (s, 1H), 4.76 (s, 2H), 4.11 (s, 3H).

### Example 171. Preparation of 3-(5,6-difluoropyridin-3-yl)-1-(2-methoxypyrimidin-5-yl)-1-((5-(trifluoromethyl)-1H-pyrazol-3-yl)methyl)urea

[M+H⁺]=430. 1,

¹H NMR (400 MHz, CDCl₃) δ 12.40-10.27 (m, 1H), 8.33 (s, 2H), 8.08 (ddd, J=2.3 Hz, 8.3 Hz, 10.6 Hz, 1H), 7.80 (br s, 1H), 6.92 (br s, 1H), 6.36 (s, 1H), 4.74 (s, 2H), 4.03 (s, 3H), 2.17 (s, 1H), 2.00 (s, 1H).

### Example 172. Preparation of 3-(4,5-difluoropyridin-2-yl)-1-(2-methoxypyrimidin-5-yl)-1-((5-(trifluoromethyl)-1H-pyrazol-3-yl)methyl)urea

[M+H⁺]=429.9,

¹H NMR (400 MHz, CDCl₃) δ 11.66-11.03 (m, 1H), 8.39 (s, 2H), 8.12-8.01 (m, 2H), 6.97-6.87 (m, 1H), 6.43-6.30 (m, 1H), 4.82-4.61 (m, 2H), 4.18-4.05 (m, 3H).

### Example 173. Preparation of 3-(3,4-difluoropyridin-2-yl)-1-(2-methoxypyrimidin-5-yl)-1-((5-(trifluoromethyl)-1H-pyrazol-3-yl)methyl)urea

[M+H]+=429.9,

¹H NMR (400 MHz, CDCl₃) 511.88-11.27 (m, 1H), 8.43 (s, 2H), 8.08 (t, J=6.0 Hz, 1H), 7.07-7.02 (m, 1H), 6.84-6.77 (m, 1H), 6.40-6.35 (m, 1H), 4.85-4.74 (m, 2H), 4.10-4.01 (m, 3H).

### Example 174. Preparation of 3-(5-(difluoromethyl)pyridin-3-yl)-1-(2-methoxypyrimidin-5-yl)-1-((5-(trifluoromethyl)-1H-pyrazol-3-yl)methyl)urea

[M+H⁺]=444.2,

¹H NMR (500 MHz, DMSO-d₆) δ 13.56 (s, 1H), 8.80 (s, 1H), 8.66 (s, 1H), 8.59 (s, 2H), 8.39 (s, 1H), 8.12 (s, 1H), 7.14 (t, J=55.3 Hz, 1H), 6.65 (s, 1H), 4.93 (s, 2H), 3.96 (s, 3H).

### Example 175. Preparation of 3-(2-(difluoromethyl)pyridin-4-yl)-1-(2-methoxypyrimidin-5-yl)-1-((5-(trifluoromethyl)-1H-pyrazol-3-yl)methyl)urea

[M+H⁺]=444.2,

¹H NMR (500 MHz, DMSO-d₆) δ 13.57 (s, 1H), 8.88 (s, 1H), 8.59 (s, 2H), 8.44 (d, J=5.6 Hz, 1H), 7.84 (s, 1H), 7.69 (d, J=5.6 Hz, 1H), 6.86 (t, J=55.2 Hz, 1H), 6.65 (s, 1H), 4.94 (s, 2H), 3.96 (s, 3H).

### Example 176. Preparation of 3-(3-(difluoromethyl)phenyl)-1-(2-methoxypyrimidin-5-yl)-1-((5-(trifluoromethyl)-1H-pyrazol-3-yl)methyl)urea

[M+H⁺]=443,

¹H NMR (400 MHz, Chloroform-d) δ 11.36 (s, 1H), 8.42 (s, 2H), 7.51 (s, 1H), 7.43 (d, J=6.2 Hz, 2H), 6.64 (t, J=56.4 Hz, 1H), 6.37 (s, 1H), 6.18 (s, 1H), 4.78 (s, 2H), 4.12 (s, 3H).

### Example 177. Preparation of 3-(6-(difluoromethyl)pyridin-2-yl)-1-(2-methoxypyrimidin-5-yl)-1-((5-(trifluoromethyl)-1H-pyrazol-3-yl)methyl)urea

[M+H⁺]=444.2,

¹H NMR (500 MHz, DMSO-d₆) δ 13.56 (s, 1H), 9.41 (s, 1H), 8.53 (d, J=1.4 Hz, 2H), 8.06 (d, J=8.5 Hz, 1H), 7.93 (t, J=8.0 Hz, 1H), 7.31 (d, J=7.4 Hz, 1H), 6.79 (t, J=55.0 Hz, 1H), 6.65 (s, 1H), 4.95 (s, 2H), 3.94 (s, 3H).

### Example 178. Preparation of 1-((5-(difluoromethyl)-1H-pyrazol-3-yl)methyl)-3-(3-(difluoromethyl)phenyl)-1-(2-methoxypyrimidin-5-yl)urea

[M+H⁺]=425.2,

¹H NMR (400 MHz, DMSO-d₆) δ 13.25 (br s, 1H), 8.53-8.47 (m, 3H), 7.69 (s, 1H), 7.63 (d, J=8.6 Hz, 1H), 7.38 (t, J=7.9 Hz, 1H), 7.20-6.75 (m, 3H), 6.43 (s, 1H), 4.91 (s, 2H), 3.95 (s, 3H).

### Example 179. Preparation of 1-((5-(difluoromethyl)-1H-pyrazol-3-yl)methyl)-3-(6-(difluoromethyl)pyridin-2-yl)-1-(2-methoxypyrimidin-5-yl)urea

[M+H⁺]=426.1,

¹H NMR (400 MHz, CDCl₃) δ 8.43-8.33 (m, 2H), 8.20 (d, J=7.9 Hz, 1H), 7.85 (t, J=8.0 Hz, 1H), 7.31 (d, J=7.5 Hz, 1H), 6.88-6.55 (m, 2H), 6.53-6.23 (m, 2H), 4.77 (s, 2H), 4.10 (s, 3H).

### Example 180. Preparation of 1-((5-cyano-1H-pyrazol-3-yl)methyl)-3-(3-(difluoromethyl)phenyl)-1-(2-methoxypyrimidin-5-yl)urea

[M+H⁺]=400.0,

¹H NMR (400 MHz, DMSO-d₆) δ 13.86 (br s, 1H), 8.55 (s, 2H), 8.47 (s, 1H), 7.68 (s, 1H), 7.62 (br d, J=8.2 Hz, 1H), 7.38 (t, J=7.9 Hz, 1H), 7.17 (d, J=7.6 Hz, 1H), 7.12-6.81 (m, 2H), 4.91 (s, 2H), 3.96 (s, 3H).

### Example 181. Preparation of 3-(3-(difluoromethyl)phenyl)-1-(3-methoxybicyclo[1.1.1]pentan-1-yl)-1-((5-(trifluoromethyl)-1H-pyrazol-3-yl)methyl)urea

[M+H⁺]=431.2,

¹H NMR (400 MHz, CDCl₃) δ 11.65-10.87 (m, 1H), 7.54 (s, 1H), 7.51-7.47 (m, 1H), 7.45-7.39 (m, 1H), 7.24 (s, 1H), 6.80 (s, 1H), 6.78-6.49 (m, 1H), 6.42 (s, 1H), 4.49 (s, 2H), 3.41-3.31 (m, 3H), 2.34 (s, 6H).

### Example 182. Preparation of 1-((4-(2-ethoxyethyl)-5-(trifluoromethyl)-1H-pyrazol-3-yl)methyl)-3-(4-fluorophenyl)-1-(6-methoxypyridin-3-yl)urea

[M+H⁺]=482.2,

¹H NMR (400 MHz, CDCl₃) δ 11.36 (s, 1H), 8.04 (d, J=2.6 Hz, 1H), 7.33 (dd, J=2.8 Hz, 8.8 Hz, 1H), 7.24-7.18 (m, 2H), 7.02-6.95 (m, 2H), 6.84 (d, J=8.8 Hz, 1H), 6.04 (s, 1H), 4.75 (s, 2H), 3.98 (s, 3H), 3.31 (q, J=7.0 Hz, 2H), 3.25 (t, J=6.6 Hz, 2H), 2.50 (t, J=6.7 Hz, 2H), 1.09 (t, J=7.0 Hz, 3H).

### Example 183. Preparation of 3-(6-fluoropyridin-2-yl)-1-(2-methoxypyrimidin-5-yl)-1-((5-(trifluoromethyl)-1H-pyrazol-3-yl)methyl)urea

[M+H⁺]=412.1,

¹H NMR (400 MHz, CDCl₃) δ 11.24 (br s, 1H), 8.72 (s, 1H), 8.36 (s, 2H), 7.97 (dd, J=1.8 Hz, 8.1 Hz, 1H), 7.81 (q, J=8.2 Hz, 1H), 6.68 (s, 1H), 6.63 (dd, J=2.1 Hz, 7.9 Hz, 1H), 6.36 (s, 1H), 4.75 (s, 2H), 4.12-4.09 (m, 3H).

### Example 184. Preparation of 3-(6-fluoropyridin-3-yl)-1-(2-methoxypyrimidin-5-yl)-1-((5-(trifluoromethyl)-1H-pyrazol-3-yl)methyl)urea

[M+H⁺]=446.1,

¹H NMR (400 MHz, DMSO-d₆) δ 8.64-8.46 (m, 3H), 8.25-8.20 (m, 1H), 8.00 (ddd, J=2.8 Hz, 7.4 Hz, 8.9 Hz, 1H), 7.11 (dd, J=3.2 Hz, 8.9 Hz, 1H), 6.64 (s, 1H), 4.91 (s, 2H), 3.95 (s, 3H).

### Example 185. Preparation of 3-(3-fluorobicyclo[1.1.1]pentan-1-yl)-1-(2-methoxypyrimidin-5-yl)-1-((5-(trifluoromethyl)-1H-pyrazol-3-yl)methyl)urea

[M+H⁺]=401.1,

¹H NMR (400 MHz, CDCl₃) δ 11.36 (br s, 1H), 8.27 (s, 2H), 6.30 (s, 1H), 4.66 (s, 1H), 4.63 (s, 2H), 4.05 (s, 3H), 2.35 (d, J=2.0 Hz, 6H), 2.17 (s, 1H).

### Example 186. Preparation of 3-(2-fluoropyrimidin-5-yl)-1-(2-methoxypyrimidin-5-yl)-1-((5-(trifluoromethyl)-1H-pyrazol-3-yl)methyl)urea

[M+H⁺]=413.1,

¹H NMR (400 MHz, CDCl₃) δ 12.13-10.28 (m, 1H), 8.74 (s, 2H), 8.36 (s, 2H), 6.77 (br s, 1H), 6.37 (s, 1H), 4.76 (s, 2H), 4.05 (s, 3H).

### Example 187. Preparation of 3-(4-fluoropyridin-2-yl)-1-(2-methoxypyrimidin-5-yl)-1-((5-(trifluoromethyl)-1H-pyrazol-3-yl)methyl)urea

[M+H⁺]=412.0,

¹H NMR (400 MHz, CDCl₃) δ 11.66-11.15 (m, 1H), 8.43-8.33 (m, 2H), 8.17-8.09 (m, 1H), 7.97-7.85 (m, 1H), 7.18-6.96 (m, 1H), 6.84-6.74 (m, 1H), 6.38 (s, 1H), 4.82-4.74 (m, 2H), 4.14-4.08 (m, 3H).

### Example 188. Preparation of 3-(3-(difluoromethyl)-4-fluorophenyl)-1-(6-methoxypyridin-3-yl)-1-((5-(trifluoromethyl)-1H-pyrazol-3-yl)methyl)urea

[M+H⁺]=461.3,

¹H NMR (500 MHz, CDCl₃) δ 11.56 (s, 1H), 8.06 (d, J=2.7 Hz, 1H), 7.52-7.43 (m, 1H), 7.41-7.39 (m, 1H), 7.35 (dd, J=8.8 Hz, 2.8 Hz, 1H), 7.06 (t, J=9.2 Hz, 1H), 6.98-6.70 (m, 2H), 6.32 (s, 1H), 6.18 (s, 1H), 4.75 (s, 2H), 3.99 (s, 3H).

### Example 189. Preparation of 3-(3-(difluoromethyl)-4-fluorophenyl)-1-(2-methoxypyrimidin-5-yl)-1-((5-(trifluoromethyl)-1H-pyrazol-3-yl)methyl)urea

[M+H⁺]=461.1,

¹H NMR (300 MHz, CDCl₃) δ 11.34 (s, 1H), 8.41 (s, 2H), 7.53-7.48 (m, 2H), 7.12 (t, J=9.1 Hz, 1H), 6.88 (t, J=54.9 Hz, 1H), 6.37 (s, 1H), 6.22 (s, 1H), 4.77 (s, 2H), 4.11 (s, 3H).

### Example 190. Preparation of 1-((5-(difluoromethyl)-1H-pyrazol-3-yl)methyl)-3-(3-(difluoromethyl)-4-fluorophenyl)-1-(2-methoxypyrimidin-5-yl)urea

[M+H⁺]=442.3,

¹H NMR (300 MHz, CDCl₃) δ 11.19 (s, 1H), 8.41 (s, 2H), 7.56-7.41 (m, 2H), 7.11 (t, J=9.3 Hz, 1H), 6.80 (td, J=54.9 Hz, 46.4 Hz, 2H), 6.34 (s, 1H), 6.21 (s, 1H), 4.77 (s, 2H), 4.11 (s, 3H).

### Example 191. Preparation of 1-((5-(difluoromethyl)-1H-pyrazol-3-yl)methyl)-3-(3-(difluoromethyl)-4-fluorophenyl)-1-(6-methoxypyridin-3-yl)urea

[M+H⁺]=443.2,

¹H NMR (400 MHz, CDCl₃) δ 11.18 (s, 1H), 8.06 (d, J=2.1 Hz, 1H), 7.54-7.45 (m, 1H), 7.41 (d, J=5.2 Hz, 1H), 7.35 (dd, J=8.7 Hz, 2.4 Hz, 1H), 7.07 (t, J=9.1 Hz, 1H), 6.97-6.55 (m, 3H), 6.29 (s, 1H), 6.14 (s, 1H), 4.73 (s, 2H), 4.00 (s, 2H).

### Example 192. Preparation of 3-(4-(difluoromethyl)-5-fluoropyridin-2-yl)-1-(2-methoxypyrimidin-5-yl)-1-((5-(trifluoromethyl)-1H-pyrazol-3-yl)methyl)urea

[M+H⁺]=463.3,

¹H NMR (500 MHz, DMSO-d₆) δ 13.56 (s, 1H), 9.51 (s, 1H), 8.54 (s, 2H), 8.41 (s, 1H), 8.17 (d, J=5.1 Hz, 1H), 7.32 (t, J=53.6 Hz, 1H), 6.65 (s, 1H), 4.95 (s, 2H), 3.96 (s, 3H).

### Example 193. Preparation of 3-(3-(difluoromethyl)-4-fluorophenyl)-1-((4-(2-hydroxyethyl)-5-(trifluoromethyl)-1H-pyrazol-3-yl)methyl)-1-(2-methoxypyrimidin-5-yl)urea

[M+H⁺]=505.2,

¹H NMR (400 MHz, CDCl₃) δ 11.42-11.22 (br, 1H), 8.38 (s, 2H), 7.59-7.44 (m, 2H), 7.08 (t, J=6.8 Hz, 1H), 6.84 (t, J=54.5 Hz, 1H), 6.25 (s, 1H), 4.81 (s, 2H), 4.07 (s, 3H), 3.62 (t, J=8.0 Hz, 2H), 2.53 (t, J=8.0 Hz, 2H).

### Example 194. Preparation of 1-((4-bromo-5-(trifluoromethyl)-1H-pyrazol-3-yl)methyl)-3-(3-(difluoromethyl)-4-fluorophenyl)-1-(2-methoxypyrimidin-5-yl)urea

[M+H⁺]=539.1, 541.1,

¹H NMR (400 MHz, CDCl₃) δ 11.57 (br s, 1H), 8.44 (s, 2H), 7.52-7.41 (m, 2H), 7.09 (t, J=9.1 Hz, 1H), 6.99-6.70 (m, 1H), 6.18 (s, 1H), 4.77 (s, 2H), 4.10 (s, 3H).

### Example 195. Preparation of 1-((5-(difluoromethyl)-1H-pyrazol-3-yl)methyl)-3-(3-(difluoromethyl)-4-fluorophenyl)-1-(6-methoxypyridazin-3-yl)urea

[M+H⁺]=443.1,

¹H NMR (400 MHz, DMSO-d₆) δ 13.34 (s, 1H), 9.43 (s, 1H), 8.53 (d, J=10.0 Hz, 1H), 7.99 (s, 1H), 7.74 (s, 1H), 7.32-6.82 (m, 4H), 6.61 (s, 1H), 5.42 (s, 2H), 3.83 (s, 3H).

### Example 196. Preparation of 1-((5-(difluoromethyl)-1H-pyrazol-3-yl)methyl)-3-(6-(difluoromethyl)-5-fluoropyridin-2-yl)-1-(2-methoxypyrimidin-5-yl)urea

[M+H⁺]=444.1,

¹H NMR (400 MHz, CDCl₃) δ 11.66-10.69 (m, 1H), 8.41-8.36 (m, 2H), 8.31-8.24 (m, 1H), 7.57 (t, J=8.9 Hz, 1H), 7.02-6.95 (m, 1H), 6.86-6.50 (m, 2H), 6.34 (s, 1H), 4.78 (s, 2H), 4.14-4.07 (m, 3H).

### Example 197. Preparation of 1-((4-bromo-5-(difluoromethyl)-1H-pyrazol-3-yl)methyl)-3-(3-(difluoromethyl)-4-fluorophenyl)-1-(2-methoxypyrimidin-5-yl)urea

[M+H⁺]=521.0, 523.0,

¹H NMR (400 MHz, DMSO-d₆) δ 13.77 (s, 1H), 8.50 (s, 2H), 8.46 (s, 1H), 7.70 (d, J=6.0 Hz, 1H), 7.67-7.59 (m, 1H), 7.35-6.78 (m, 3H), 4.89 (s, 2H), 3.98-3.88 (m, 3H).

### Example 198. Preparation of 1-((5-(difluoromethyl)-4-(2-hydroxypropan-2-yl)-1H-pyrazol-3-yl)methyl)-3-(3-(difluoromethyl)-4-fluorophenyl)-1-(2-methoxypyrimidin-5-yl)urea

[M+H⁺]=501.2,

¹H NMR (400 MHz, CDCl₃) δ 11.47-10.67 (m, 1H), 8.34 (s, 2H), 7.49-7.37 (m, 2H), 7.01 (t, J=9.2 Hz, 1H), 6.94-6.59 (m, 2H), 6.33 (s, 1H), 5.07 (s, 2H), 3.99 (s, 3H), 1.64 (s, 1H), 1.43 (s, 6H).

### Example 199. Preparation of 3-(3-(difluoromethyl)-4-fluorophenyl)-1-((4-(2-hydroxypropyl)-5-(trifluoromethyl)-1H-pyrazol-3-yl)methyl)-1-(2-methoxypyrimidin-5-yl)urea

[M+H⁺]=519.2,

¹H NMR (400 MHz, CDCl₃) δ 11.89-11.03 (m, 1H), 8.38 (s, 2H), 7.47 (s, 2H), 7.08 (s, 1H), 7.01-6.65 (m, 1H), 6.34 (s, 1H), 5.07-4.58 (m, 2H), 4.07 (s, 3H), 3.81 (s, 1H), 2.57 (d, J=14.9 Hz, 1H), 2.24 (s, 1H), 1.14 (s, 3H).

### Example 200. Preparation of 3-(3-(difluoromethyl)-4-fluorophenyl)-1-(6-methoxypyridazin-3-yl)-1-((5-(trifluoromethyl)-1H-pyrazol-3-yl)methyl)urea

[M+H⁺]=461.2,

¹H NMR (400 MHz, DMSO-d₆) δ 13.68 (s, 1H), 9.42 (s, 1H), 8.54 (d, J=10.0 Hz, 1H), 7.99 (s, 1H), 7.74 (s, 1H), 7.35-7.17 (m, 3H), 6.83 (s, 1H), 5.45 (s, 2H), 3.84 (s, 3H).

### Example 201. Preparation of 1-((5-(difluoromethyl)-1H-pyrazol-3-yl)methyl)-3-(3-(difluoromethyl)-4-fluorophenyl)-1-(6-methoxypyridazin-4-yl)urea

[M+H⁺]=443.0,

¹H NMR (400 MHz, DMSO-d₆) δ 13.46-13.15 (m, 1H), 9.68-9.25 (m, 1H), 8.96 (d, J=2.3 Hz, 1H), 7.77 (dd, J=2.2 Hz, 6.4 Hz, 1H), 7.63 (dd, J=3.7 Hz, 8.4 Hz, 1H), 7.33 (d, J=9.2 Hz, 1H), 7.30-6.75 (m, 3H), 6.41 (s, 1H), 5.09 (s, 2H), 4.01 (s, 3H).

### Example 202. Preparation of 3-(6-(difluoromethyl)-5-fluoropyridin-2-yl)-1-(2-methoxypyrimidin-5-yl)-1-((5-(trifluoromethyl)-1H-pyrazol-3-yl)methyl)urea

[M+H⁺]=462.1,

¹H NMR (400 MHz, CDCl₃) δ 11.32 (s, 1H), 8.41 (s, 2H), 8.28 (dd, J=9.2 Hz, 2.8 Hz, 1H), 7.60 (t, J=8.9 Hz, 1H), 7.00 (s, 1H), 6.68 (t, J=53.7 Hz, 1H), 6.39 (s, 1H), 4.79 (s, 2H), 4.13 (s, 3H).

### Example 203. Preparation of 3-(3-(difluoromethyl)-4-fluorophenyl)-1-(6-methoxypyridazin-4-yl)-1-((5-(trifluoromethyl)-1H-pyrazol-3-yl)methyl)urea

¹H NMR (400 MHz, CDCl₃) δ 8.76 (d, J=2.0 Hz, 1H), 7.66-7.59 (m, 2H), 7.41-7.30 (m, 1H), 7.14 (t, J=9.0 Hz, 1H), 7.03-6.75 (m, 2H), 6.44 (s, 1H), 4.91 (s, 2H), 4.13 (s, 3H).

### Example 204. Preparation of 3-(6-(difluoromethyl)-5-fluoropyridin-2-yl)-1-(2-methoxypyrimidin-5-yl)-1-((5-(trifluoromethyl)-1H-pyrazol-3-yl)methyl)urea

[M+H⁺]=462.0,

¹H NMR (400 MHz, Acetonitrile-d₃) δ 11.68-11.54 (m, 1H), 8.39 (s, 2H), 8.21 (dd, J=3.4 Hz, 9.2 Hz, 1H), 7.67 (t, J=9.2 Hz, 1H), 7.59 (s, 1H), 7.53 (s, 1H), 6.88-6.57 (m, 1H), 6.51 (s, 1H), 4.88 (s, 2H), 4.00 (s, 3H).

### Example 205. Preparation of 3-(3-(difluoromethyl)-4-fluorophenyl)-1-((4-(2-hydroxy-2-methylpropyl)-5-(trifluoromethyl)-1H-pyrazol-3-yl)methyl)-1-(2-methoxypyrimidin-5-yl)urea

[M+H⁺]=533.0,

¹H NMR (400 MHz, CDCl₃) δ 8.39-8.31 (m, 2H), 7.55-7.49 (m, 1H), 7.49-7.44 (m, 1H), 7.10 (t, J=9.2 Hz, 1H), 7.01-6.71 (m, 1H), 6.35-6.27 (m, 1H), 4.96-4.91 (m, 2H), 4.08 (s, 3H), 2.50-2.46 (m, 2H), 1.16-1.12 (m, 6H).

### Example 206. Preparation of 3-(4-(difluoromethyl)-3-fluorophenyl)-1-(2-methoxypyrimidin-5-yl)-1-((5-(trifluoromethyl)-1H-pyrazol-3-yl)methyl)urea

[M+H⁺]=461.1,

¹H NMR (400 MHz, Chloroform-d) δ 11.29 (s, 1H), 8.41 (s, 2H), 7.59-7.43 (m, 2H), 7.15-6.65 (m, 2H), 6.36 (d, J=10.8 Hz, 2H), 4.78 (s, 2H), 4.11 (s, 3H).

### Example 207. Preparation of 3-(3-(difluoromethyl)-4-fluorophenyl)-1-((4-(2-hydroxypropan-2-yl)-5-(trifluoromethyl)-1H-pyrazol-3-yl)methyl)-1-(2-methoxypyrimidin-5-yl)urea

[M+H⁺]=519.2,

¹H NMR (400 MHz, CDCl₃) δ 11.37 (s, 1H), 8.38 (s, 2H), 7.55-7.49 (m, 1H), 7.46 (dd, J=2.6 Hz, 5.8 Hz, 1H), 7.08 (t, J=9.1 Hz, 1H), 7.00-6.68 (m, 1H), 6.32 (s, 1H), 5.20 (s, 2H), 4.06 (s, 3H), 1.50 (s, 1H), 1.47 (s, 6H).

### Example 208. Preparation of 1-((5-(difluoromethyl)-4-(2-hydroxy-2-methylpropyl)-1H-pyrazol-3-yl)methyl)-3-(3-(difluoromethyl)-4-fluorophenyl)-1-(2-methoxypyrimidin-5-yl)urea

[M+H⁺]=515.0,

¹H NMR (400 MHz, CDCl₃) δ 8.39-8.35 (m, 2H), 7.58-7.47 (m, 2H), 7.10 (t, J=9.2 Hz, 1H), 7.03-6.63 (m, 2H), 6.48-6.38 (m, 1H), 4.96-4.91 (m, 2H), 4.10-4.07 (m, 3H), 2.56-2.51 (m, 2H), 1.21-1.13 (m, 6H).

### Example 209. Preparation of 1-((5-cyano-1H-pyrazol-3-yl)methyl)-3-(3-(difluoromethyl)-4-fluorophenyl)-1-(2-methoxypyrimidin-5-yl)urea

[M+H⁺]=418.2,

¹H NMR (500 MHz, DMSO-d₆) δ 13.87 (s, 1H), 8.57 (s, 2H), 8.48 (s, 1H), 7.78-7.69 (m, 1H), 7.65 (dd, J=8.7 Hz, 4.1 Hz, 1H), 7.30-7.08 (m, 2H), 6.90 (s, 1H), 4.91 (s, 2H), 3.96 (s, 3H).

### Example 210. Preparation of 1-((5-(difluoromethyl)-1H-pyrazol-3-yl)methyl)-3-(6-(difluoromethyl)-5-fluoropyridin-2-yl)-1-(2-methoxypyrimidin-5-yl)urea

[M+H⁺]=444.2,

¹H NMR (500 MHz, DMSO-d₆) δ 13.18 (s, 1H), 9.46 (s, 1H), 8.49 (s, 2H), 8.10 (dd, J=9.4 Hz, 3.1 Hz, 1H), 7.91 (t, J=9.4 Hz, 1H), 6.96 (td, J=54.8 Hz, 54.0 Hz, 45.8 Hz, 2H), 6.43 (s, 1H), 4.93 (s, 2H), 3.94 (s, 3H).

### Example 211. Preparation of 3-(2-(difluoromethyl)-3-fluoropyridin-4-yl)-1-(2-methoxypyrimidin-5-yl)-1-((5-(trifluoromethyl)-1H-pyrazol-3-yl)methyl)urea

[M+H⁺]=462.04,

¹H NMR (500 MHz, Methanol-d₄) δ 8.50 (s, 2H), 8.34 (d, J=5.4 Hz, 1H), 8.14 (t, J=5.7 Hz, 1H), 6.88 (t, J=53.4 Hz, 1H), 5.02 (s, 2H), 4.07 (s, 3H).

### Example 212. Preparation of 1-((5-cyano-1H-pyrazol-3-yl)methyl)-3-(6-(difluoromethyl)-5-fluoropyridin-2-yl)-1-(2-methoxypyrimidin-5-yl)urea

[M+H⁺]=418.9,

¹H NMR (400 MHz, DMSO-d₆) δ 13.81 (br s, 1H), 9.50 (br s, 1H), 8.52 (s, 2H), 8.08 (dd, J=3.5 Hz, 9.2 Hz, 1H), 7.91 (t, J=9.4 Hz, 1H), 7.00 (br t, J=53.2 Hz, 1H), 6.89 (s, 1H), 4.93 (s, 2H), 3.94 (s, 3H).

### Example 213. Preparation of 3-(3-(difluoromethyl)-4-fluorophenyl)-1-(3-methoxybicyclo[1.1.1]pentan-1-yl)-1-((5-(trifluoromethyl)-1H-pyrazol-3-yl)methyl)urea

[M+H⁺]=449.2,

¹H NMR (400 MHz, CDCl₃) δ 11.31 (br s, 1H), 7.62-7.53 (m, 1H), 7.50 (dd, J=2.6 Hz, 5.9 Hz, 1H), 7.12 (t, J=9.1 Hz, 1H), 7.04-6.72 (m, 2H), 6.42 (s, 1H), 4.49 (s, 2H), 3.38-3.29 (m, 3H), 2.38-2.32 (m, 6H).

### Example 214. Preparation of 3-(6-(difluoromethyl)-5-fluoropyridin-2-yl)-1-(3-methoxybicyclo[1.1.1]pentan-1-yl)-1-((5-(trifluoromethyl)-1H-pyrazol-3-yl)methyl)urea

[M+H⁺]=450.2,

¹H NMR (400 MHz, CDCl₃) δ 11.28 (s, 1H), 8.18 (dd, J=3.4 Hz, 9.2 Hz, 1H), 7.62-7.49 (m, 2H), 6.87-6.52 (m, 1H), 6.44 (s, 1H), 4.51 (s, 2H), 3.35 (s, 3H), 2.38 (s, 6H).

### Example 215. Preparation of 1-((5-cyano-1H-pyrazol-3-yl)methyl)-3-(4-fluoro-3-(trifluoromethyl)phenyl)-1-(2-methoxypyrimidin-5-yl)urea

[M+H⁺]=436.1,

¹H NMR (400 MHz, CDCl₃) δ 8.40-8.35 (m, 2H), 7.63-7.57 (m, 1H), 7.55-7.50 (m, 1H), 7.16 (t, J=9.3 Hz, 1H), 6.52-6.47 (m, 1H), 6.42-6.38 (m, 1H), 4.77-4.72 (m, 2H), 4.11-4.06 (m, 3H).

### Example 216. Preparation of 1,3-bis(2-methoxypyrimidin-5-yl)-1-((5-(trifluoromethyl)-1H-pyrazol-3-yl)methyl)urea

[M+H⁺]=425.2,

¹H NMR (400 MHz, DMSO-d₆) δ 13.61 (br s, 1H), 8.58 (d, J=4.8 Hz, 4H), 6.63 (s, 1H), 4.92 (s, 2H), 3.95 (s, 3H), 3.87 (s, 3H).

### Example 217. Preparation of 1-(2-methoxypyrimidin-5-yl)-3-(3-methylbicyclo[1.1.1]pentan-1-yl)-1-((5-(trifluoromethyl)-1H-pyrazol-3-yl)methyl)urea

[M+H⁺]=397.2,

¹H NMR (400 MHz, CDCl₃) δ 11.91-10.92 (m, 1H), 8.27 (s, 2H), 6.28 (s, 1H), 4.61 (s, 2H), 4.58 (s, 1H), 4.06 (s, 3H), 2.17 (s, 1H), 1.87 (s, 6H), 1.22 (s, 3H).

### Example 218. Preparation of 1-(2-methoxypyrimidin-5-yl)-3-(2-(methylthio)pyrimidin-5-yl)-1-((5-(trifluoromethyl)-1H-pyrazol-3-yl)methyl)urea

[M+H⁺]=441.1,

¹H NMR (400 MHz, CDCl₃) δ 8.56 (s, 2H), 8.26 (s, 2H), 7.19 (s, 1H), 6.90 (s, 1H), 6.27 (s, 1H), 4.68 (s, 2H), 3.93 (s, 3H), 2.48 (s, 3H).

### Example 219. Preparation of 1-(2-methoxypyrimidin-5-yl)-1-((5-(trifluoromethyl)-1H-pyrazol-3-yl)methyl)-3-(3,4,5-trifluorophenyl)urea

[M+H⁺]=447. 1,

¹H NMR (400 MHz, CDCl₃) δ 8.27 (s, 2H), 7.07-6.97 (m, 2H), 6.33 (s, 1H), 6.28 (s, 1H), 4.66 (s, 2H), 4.00 (s, 3H).

### Example 220. Preparation of 1-((5-(difluoromethyl)-1H-pyrazol-3-yl)methyl)-1-(2-methoxypyrimidin-5-yl)-3-(3,4,5-trifluorophenyl)urea

### Preparation of 1,2,3-trifluoro-5-isocyanatobenzene

A mixture of bis(trichloromethyl) carbonate (81 mg, 272 μmol, 0.4 eq) in toluene (2 mL) was stirred at 25° C. for 10 min, then a mixture of 3,4,5-trifluoroaniline (100 mg, 680 μmol, 1 eq) in toluene (1 mL) was added, and the resulting mixture was stirred at 120° C. for 1 hr to afford a solution of 1,2,3-trifluoro-5-isocyanatobenzene in toluene (3 mL), which was used in the next step without any purification.

### Preparation of 1-((5-(difluoromethyl)-1-(tetrahydro-2H-pyran-2-yl)-1H-pyrazol-3-yl)methyl)-1-(2-methoxypyrimidine-5-yl)-3-(3,4,5-trifluorophenyl)urea

To a mixture of N-((5-(difluoromethyl)-1-(tetrahydro-2H-pyran-2-yl)-1H-pyrazol-3-yl)methyl)-2-methoxypyrimidin-5-amine (100 mg, 295 μmol, 1 eq) in ACN (5 mL) was added the solution of 1,2,3-trifluoro-5-isocyanatobenzene in toluene obtained in the previous step. DMAP (108 mg, 884 μmol, 3 eq) was added to the mixture, and the resulting mixture was stirred at 25° C. for 16 hr. After the reaction mixture was concentrated, the residue was purified using silica gel chromatography (petroleum ether:ethyl acetate=10:1 to 1:1) to afford 1-((5-(difluoromethyl)-1-(tetrahydro-2H-pyran-2-yl)-1H-pyrazol-3-yl)methyl)-1-(2-methoxypyrimidine-5-yl)-3-(3,4,5-trifluorophenyl)urea (91 mg, 60% yield) as a yellow oil.

### Preparation of 1-((5-(difluoromethyl)-1H-pyrazol-3-yl)methyl)-1-(2-methoxypyrimidin-5-yl)-3-(3,4,5-trifluorophenyl)urea

To a mixture of 1-((5-(difluoromethyl)-1-(tetrahydro-2H-pyran-2-yl)-1H-pyrazol-3-yl)methyl)-1-(2-methoxypyrimidine-5-yl)-3-(3,4,5-trifluorophenyl)urea (91 mg, 178 μmol, 1 eq) in DCM (6 mL) was added TFA (2 mL), and the mixture was stirred at 25° C. for 1 hr. After the reaction mixture was concentrated, the residue was purified by prep-HPLC (column: Unisil 3-100 C18 Ultra 150 mm×50 mm×3 μm; mobile phase: [water (0.225% FA)-ACN]; B %: 43%-63%, 10 min), followed by lyophilization to afford 1-((5-(difluoromethyl)-1H-pyrazol-3-yl)methyl)-1-(2-methoxypyrimidin-5-yl)-3-(3,4,5-trifluorophenyl)urea (29.5 mg, 38.4% yield, 99% purity) as an off-white solid.

[M+H⁺]=429.1,

¹H NMR (400 MHz, CDCl₃) δ 11.65-10.38 (m, 1H), 8.31 (s, 2H), 7.18-7.05 (m, 2H), 6.88-6.50 (m, 2H), 6.31 (s, 1H), 4.73 (s, 2H), 4.04 (s, 3H).

### Example 221. Preparation of 1-(2-methoxypyrimidin-5-yl)-1-((5-(trifluoromethyl)-1H-pyrazol-3-yl)methyl)-3-(3,5,6-trifluoropyridin-2-yl)urea

[M+H⁺]=448.2,

¹H NMR (400 MHz, DMSO-d₆) δ 13.63 (s, 1H), 9.14 (s, 1H), 8.48 (s, 2H), 8.37-8.28 (m, 1H), 6.61 (s, 1H), 4.90 (s, 2H), 3.93 (s, 3H).

### Example 222. Preparation of 1-(3-methoxybicyclo[1.1.1]pentan-1-yl)-1-((5-(trifluoromethyl)-1H-pyrazol-3-yl)methyl)-3-(3,4,5-trifluorophenyl)urea

[M+H⁺]=435.2,

¹H NMR (400 MHz, CDCl₃) δ 11.70-10.70 (m, 1H), 7.14-7.05 (m, 2H), 6.71 (s, 1H), 6.42 (s, 1H), 4.48 (s, 2H), 3.34 (s, 3H), 2.37-2.28 (m, 6H).

### Example 223. Preparation of 1-((5-cyano-1H-pyrazol-3-yl)methyl)-1-(2-methoxypyrimidin-5-yl)-3-(3,4,5-trifluorophenyl)urea

[M+H⁺]=404.0,

¹H NMR (400 MHz, DMSO-d₆) δ 13.84 (s, 1H), 8.54 (s, 3H), 7.45-7.37 (m, 2H), 6.89 (s, 1H), 4.89 (s, 2H), 3.96 (s, 3H).

### Example 224. Preparation of 1-(2-methoxypyrimidin-5-yl)-1-((5-(trifluoromethyl)-1H-pyrazol-3-yl)methyl)-3-(4,5,6-trifluoropyridin-2-yl)urea

### Preparation of 2-methoxypyrimidin-5-yl((1-(tetrahydro-2H-pyran-2-yl)-5-(trifluoromethyl)-1H-pyrazol-3-yl)methyl)carbamoyl chloride

To a solution of triphosgene (145 mg, 490 μmol, 0.35 eq) in DCM (10 mL) was added 2-methoxy-N-((1-(tetrahydro-2H-pyran-2-yl)-5-(trifluoromethyl)-1H-pyrazol-3-yl)methyl)pyrimidin-5-amine (0.5 g, 1.40 mmol, 1 eq) in portions at 0° C. Then DIPEA (543 mg, 4.20 mmol, 731 μL, 3 eq) was added dropwise, and the resulting mixture was stirred at 15° C. for 1 hr. The mixture was poured into a HCl (0.5 M, 10 mL) aqueous solution, followed by extraction with DCM (30 mL×3). The combined organic layer was washed with a saturated NaHCO₃ solution (20 mL), dried over Na₂SO₄, and filtered. The filtrate was concentrated in vacuo to afford 2-methoxypyrimidin-5-yl((1-(tetrahydro-2H-pyran-2-yl)-5-(trifluoromethyl)-1H-pyrazol-3-yl)methyl)carbamoyl chloride (0.62 g, crude) as a yellow gum.

### Preparation of 1-(2-methoxypyrimidin-5-yl)-1-((1-(tetrahydro-2H-pyran-2-yl)-5-(trifluoromethyl)-1H-pyrazole-3-yl)methyl)-3-(4,5,6-trifluoropyridin-2-yl)urea

To a solution of 4,5,6-trifluoropyridin-2-amine (65 mg, 439 μmol, 1 eq) in THF (2 mL) was added NaH (35 mg, 878 μmol, 60% in oil, 2 eq) batchwise at 0° C. The resulting mixture was stirred at 20° C. for 30 min before the addition of 2-methoxypyrimidin-5-yl((1-(tetrahydro-2H-pyran-2-yl)-5-(trifluoromethyl)-1H-pyrazol-3-yl)methyl)carbamoyl chloride obtained in the previous step (129 mg, 307 μmol, 0.7 eq). The mixture was stirred at 20° C. for 1 hr before being poured into an NH₄Cl aqueous solution (15 mL), followed by extraction with ethyl acetate (15 mL×3). The combined organic layer was washed with brine (10 mL), dried over anhydrous Na₂SO₄, and filtered. The filtrate was concentrated in vacuo. The crude product was purified by reversed-phase MPLC (0.1% FA condition) to afford 1-(2-methoxypyrimidin-5-yl)-1-((1-(tetrahydro-2H-pyran-2-yl)-5-(trifluoromethyl)-1H-pyrazole-3-yl)methyl)-3-(4,5,6-trifluoropyridin-2-yl)urea (90 mg, 39% yield) as a yellow solid.

### Preparation of 1-(2-methoxypyrimidin-5-yl)-1-((5-(trifluoromethyl)-1H-pyrazol-3-yl)methyl)-3-(4,5,6-trifluoropyridin-2-yl)urea

A solution of 1-(2-methoxypyrimidin-5-yl)-1-((1-(tetrahydro-2H-pyran-2-yl)-5-(trifluoromethyl)-1H-pyrazole-3-yl)methyl)-3-(4,5,6-trifluoropyridin-2-yl)urea (90 mg, 169 μmol, 1 eq) in TFA (0.5 mL) and DCM (2 mL) was stirred at 20° C. for 1 hr. The solution was concentrated to dryness. The crude product was purified by prep-HPLC (column: Shim-pack C18 150×25×10 μm; mobile phase: [water (0.225% FA)-ACN]; B %: 48%-68%, 10 min), followed by lyophilization to afford 1-(2-methoxypyrimidin-5-yl)-1-((5-(trifluoromethyl)-1H-pyrazol-3-yl)methyl)-3-(4,5,6-trifluoropyridin-2-yl)urea (54.2 mg, 71% yield, 99% purity) as a white solid.

[M+H⁺]=448.2,

¹H NMR (400 MHz, CDCl₃) δ 11.22 (s, 1H), 8.37 (s, 2H), 7.94 (dd, J=4.2 Hz, 10.7 Hz, 1H), 6.69 (s, 1H), 6.38 (s, 1H), 4.76 (s, 2H), 4.11 (s, 3H).

### Example 225. Preparation of 1-(2-methoxypyrimidin-5-yl)-1-((5-(trifluoromethyl)-1H-pyrazol-3-yl)methyl)-3-(6-(trifluoromethyl)pyridin-2-yl)urea

[M+H⁺]=462.1,

¹H NMR (500 MHz, CDCl₃) δ 11.32 (s, 1H), 8.39 (s, 2H), 8.30 (d, J=8.5 Hz, 1H), 7.89 (t, J=8.0 Hz, 1H), 7.39 (d, J=7.5 Hz, 1H), 6.98 (s, 1H), 6.37 (s, 1H), 4.77 (s, 2H), 4.11 (s, 4H).

### Example 226. Preparation of 3-(3-cyano-4-fluorophenyl)-1-(2-methoxypyrimidin-5-yl)-1-((1-propionyl-5-(trifluoromethyl)-1H-pyrazol-3-yl)methyl)urea

¹H NMR (300 MHz, DMSO) δ 7.90 (br, 1H), 7.73 (dd, J=6.90 Hz, 2.61 Hz, 1H), 7.40 (m, 2H), 7.27 (t, J=9.15 Hz, 1H), 7.07 (d, J=8.91 Hz, 2H), 6.96 (d, J=8.94 Hz, 2H), 4.56 (s, 2H), 3.76 (s, 3H), 3.66 (s, 3H), 1.80 (s, 3H).

### Experimental Example 1: Culturing of Cell and Treatment with Compound

HepAD38 cells were cultured in DMEM medium (Welgene) supplemented with 200 unit/mL penicillin, 200 μg/mL streptomycin, and 10% FBS. During maintenance and passage, the cells were cultured together with 0.4 μg/mL tetracycline. Two days before the treatment with compounds for inducing HBV replication was started, the medium was replaced with a tetracycline-free complete growth medium. The HepAD38 cells were seeded in a 48-well culture plate at a density of 1×10⁵ cells/well and treated with DMSO (0.2%, control) or a test compound (final concentration ranging from 1.5 nM to 0.37 μM).

### Experimental Example 2: Real-Time PCR for Intracellular HBV DNA

HepAD38 cells were treated with a compound for 65 hours and then recovered, and intracellular HBV DNA was extracted therefrom according to the protocol in the DNeasy Blood & Tissue Kit (Qiagen). The primers and probes used to quantify HBV DNA were 5′-CTCGTGGTGGACTTCTCTC-3′, 5′-CTGCAGGATGAAGAGGAA-3′, and 5′-/56-FAM/TGT CCT GGT/ZEN/TAT CGC TGG ATG TGT CT/3IABkFQ/-3′. The HBV DNA was amplified by real-time PCR assay using LightCycler 480 (Roche) (J. Virol., 2018, 92(16):e00339-18). All of the processes were confirmed in duplicate.

### Experimental Example 3: Cell Viability Assay

HepAD38 cells were cultured for 2 days in Dulbecco's Modified Eagle's medium (Welgene, LM001-05) supplemented with 10% FBS without tetracycline. The cells were seeded in 48-well culture plates (1×10⁵ cells/well) and treated with each compound at five concentrations (0 μM (0.8% DMSO as a control), 33 μM, 50 μM, 66 μM, and 100 μM). After 65 hours of treatment, viability was measured using EZ-Cytox cell viability assay kit (Daeil Lab Service) according to the manufacturer's instructions. The absorbance was measured at 450 nm using a spectrophotometer (Spark, Tecan). The IC₅₀, protein inhibition rate, cytotoxicity, and C Log P values of these compounds are presented in Table 1 below. Here, NVR-3-778 (denoted as NVR) was used as a positive control.

TABLE 1

| | | Protein | | | | |
|---|---|---|---|---|---|---|
| | | % inhi- | | Cytotoxicity | | |
| | IC₅₀ | bition | EC₅₀ | % Liv- | | |
| | (μM) | rate | (μM) | ability | | |
| Exam- | (NVR: | (@30 | (NVR: | (@100 | CC₅₀ | |
| ple | 0.34) | μM) | 3.6) | μM) | (μM) | CLogP |
| 1 | >1.1 | | | | | |
| 2 | >1.1 | | | | | |
| 3 | >1.1 | | | 74.2 | >100 | |
| 4 | 0.021 | | | | | |
| 5 | 0.38 | | | | | |
| 6 | >1.1 | | | 100.5 | >100 | |
| 7 | 0.019 | | | 59.3 | >100 | |
| 8 | >1.1 | | | | | |
| 9 | 0.31 | | | 105.0 | >100 | |
| 10 | >1.1 | | | | | |
| 11 | 0.010 | | | | | |
| 12 | 0.045 | | | | | |
| 13 | 0.21 | | | 100.0 | >100 | |
| 14 | 0.11 | 14 | 7.3 | 95.4 | >100 | |
| 15 | 0.31 | 64.7 | 1.4 | 84.0 | >100 | |
| 16 | >1.1 | | | | 97.6 | 3.04 |
| 17 | >1.1 | | | | >100 | 1.68 |
| 18 | >1.1 | | | | >100 | 3.23 |
| 19 | >1.1 | | | | >100 | 3.4 |
| 20 | >1.1 | | | | >100 | 4.03 |
| 21 | >1.1 | | | | >100 | 2.47 |
| 22 | >1.1 | | | | | |
| 23 | >1.1 | | | | >100 | 1.93 |
| 24 | 0.16 | | | 53.4 | >100 | |
| 25 | >1.1 | | | 100 | >100 | 2.82 |
| 26 | >1.1 | | | 93.0 | >100 | 3.43 |
| 27 | 0.16 | | | | | |
| 28 | 0.06 | | | 7.0 | 47.2 | |
| 29 | >1.1 | | | 6.9 | 12.2 | |
| 30 | >1.1 | | | 52.4 | >100 | |
| 31 | 0.41 | 90.4 | 3.3 | 91.2 | >100 | |
| 32 | 0.0099 | 5 | 9.4 | 4.1 | 81.4 | |
| 33 | >1.1 | 77.8 | | 96.0 | | |
| 34 | >1.1 | 55.2 | | 92.9 | | |
| 35 | >1.1 | 33.7 | | 90.5 | | |
| 36 | 0.036 | 91.6 | 9.2 | 100.2 | >100 | |
| 37 | >1.1 | 28.3 | | 22.4 | | |
| 38 | 0.018 | 80 | 1.6 | 104.0 | >100 | |
| 39 | 0.33 | | | 96.0 | >100 | |
| 40 | 0.016 | 99.5 | 3.8 | 4.6 | 82.5 | |
| 41 | 0.018 | 59* | 2.6 | 97.8 | >100 | |
| 42 | 0.037 | 13* | 4.3 | 99.5 | >100 | |
| 43 | 0.043 | 96.7 | 6.06 | 92.0 | >100 | |
| 44 | >1.1 | 58 | | 100.0 | >100 | |
| 45 | 0.012 | 95.6 | 3.3 | 3.3 | >50 | |
| 46 | 0.0094 | 92.8 | 4.3 | 100.1 | >100 | |
| 47 | 0.99 | 95.3 | 9.6 | 50.7 | >100 | |
| 48 | 1.1 | 90 | 6.4 | | >100 | 3.0 |
| 49 | >1.1 | 72 | | | >100 | 1.9 |
| 50 | 0.26 | 94.3 | 3.8 | | >100 | 4.1 |
| 51 | 0.035 | 87.1 | 3.4 | 36.7 | >50 | |
| 52 | >1.1 | 4.0 | | 100.0 | >100 | |
| 53 | >1.1 | 20.1 | | 58.9 | >100 | |
| 54 | 0.013 | 84.0 | 3.5 | 17.4 | 40.6 | |
| 55 | 0.017 | | | 102.4 | >100 | |
| 56 | 0.40 | 86.4 | 6.6 | 98.2 | >100 | |
| 57 | 0.023 | 96.2 | 2.0 | 93.2 | >100 | |
| 58 | 0.16 | 93.1 | 5.0 | 92.7 | >100 | |
| 59 | 0.013 | 94.9 | 4.0 | 91.2 | >100 | |
| 60 | 0.11 | 94.7 | 3.0 | 76.0 | >100 | |
| 61 | 0.502 | 81.8 | 4.4 | 40.8 | 98.8 | |
| 62 | 0.20 | 100.4 | 6.9 | 6.5 | 86.68 | |
| 63 | 0.014 | 90.5 | 2.6 | 94.5 | >100 | |
| 64 | 0.18 | 91.1 | 5.8 | 100.0 | >100 | |
| 65 | >1.1 | 53.5 | >30 | 112.2 | >100 | |
| 66 | >0.3 | | | | 81.9 | 5.06 |
| 67 | >0.3 | | | | >100 | 3.68 |
| 68 | 0.0093 | | | 48.9 | 98.6 | 4.46 |
| 69 | 0.021 | | | 94.5 | >100 | 3.28 |
| 70 | 0.009 | | | 98.0 | >100 | 3.28 |
| 71 | 0.076 | | | | >100 | 4.3 |
| 72 | 0.048 | | | 48.5 | >66.6 | 3.99 |
| 73 | 0.019 | | | 96.6 | >100 | 3.04 |
| 74 | 0.022 | | | 55.6 | >100 | 5.07 |
| 75 | 0.036 | | | 92.1 | >100 | 2.64 |
| 76 | | | | | | 4.19 |
| 77 | 0.036 | | | 86.7 | >100 | 3.59 |
| 78 | 0.0303 | | | 94.0 | >100 | 5.29 |
| 79 | 0.019 | | | 18.5 | | |
| 80 | 0.019 | | | 21.4 | 88.4 | 4.51 |
| 81 | 0.0145 | | | 76.0 | >100 | 4.52 |
| 82 | >1.1 | | | 85.8 | >100 | 3.69 |
| 83 | >1.1 | | | 95.1 | >100 | 5.59 |
| 84 | 0.017 | | | | | 3.43 |
| 85 | 0.23 | | | | | |
| 86 | 0.016 | | | | | |
| 87 | 0.034 | | | | | |
| 88 | >1.1 | | | 74.5 | >100 | |
| 89 | 0.05 | | | 96.0 | >100 | |
| 90 | | | | | | |
| 91 | | | | | | |
| 92 | >1.1 | | | | >100 | |
| 93 | 0.32 | | | | >100 | |
| 94 | 0.26 | | | 4.8 | 64.4 | |
| 95 | 0.83 | | | 29.9 | 62.2 | |
| 96 | >1.1 | | | 93.4 | >100 | |
| 97 | >1.1 | | | 86.2 | >100 | |
| 98 | 0.018 | | | 30.1 | >66 | |
| 99 | 0.89 | | | 83.4 | >100 | |
| 100 | 0.036 | | | | | |
| 101 | >1.1 | | | | | |
| 102 | >1.1 | | | 3.8 | >66 | |
| 103 | >1.1 | | | ND | ND | |
| 104 | 0.027 | | | | | |
| 105 | 0.034 | | | 3.5 | 55.8 | |
| 106 | 0.12 | | | | | |
| 107 | 0.062 | | | | | |
| 108 | 0.36 | 89.9 | 5.3 | | | |
| 109 | 0.15 | | | 82 | >100 | 2.83 |
| 110 | >1.1 | | | | >100 | 3.47 |
| 111 | >1.1 | | | | >100 | 1.92 |
| 112 | >1.1 | | | | >66 | 1.92 |
| 113 | >1.1 | | | 102.0 | >100 | 3.47 |
| 114 | 0.014 | 74.6 | 1.6 | 7.9 | 54.5 | |
| 115 | >1.1 | | | 84.1 | >100 | 1.28 |
| 116 | >1.1 | 69.5 | | 103.9 | >100 | |
| 117 | >1.1 | 7 | | 3.2 | >33 | |
| 118 | >1.1 | | | 31.3 | <50 | 4.50 |
| 119 | 0.32 | | | 18.8 | <50 | 4.70 |
| 120 | 0.095 | | | 92.0 | >100 | 3.45 |
| 121 | 0.013 | | | 92.8 | >100 | 3.96 |
| 122 | 0.35 | | | | >100 | 3.64 |
| 123 | 0.249 | | | | | 2.09 |
| 124 | 0.047 | | | | | |
| 125 | 0.23 | | | 109.0 | >100 | 2.1 |
| 126 | 0.54 | | | | | 3.05 |
| 127 | 0.51 | | | 95.0 | >100 | 2.09 |
| 128 | 0.34 | | | 65.9 | >100 | 2.09 |
| 129 | 0.06 | | | 93.1 | >100 | 1.89 |
| 130 | 0.065 | | | 68.0 | >100 | 4.13 |
| 131 | >1.1 | 94.7 | 3.8 | 100.3 | >100 | |
| 132 | 0.055 | 86.9 | 5.3 | 81.9 | >100 | |
| 133 | 0.11 | 5 | NA | 4.3 | 74.2 | 3.7 |
| 134 | 0.069 | 84.5 | 3.5 | 6.5 | 70.3 | 2.9 |
| 135 | 0.2 | | | | >100 | 3.34 |
| 136 | 0.048 | | | 7.3 | 64 | 3.16 |
| 137 | 0.37 | | | | | |
| 138 | >1.1 | | | 71.9 | >100 | |
| 139 | 0.04 | | | | | |
| 140 | 0.037 | | | | | |
| 141 | 0.42 | | | | | |
| 142 | 0.1 | | | 21.0 | 46.9 | |
| 143 | >1.1 | | | | | |
| 144 | 0.22 | | | 6.3 | 59.9 | |
| 145 | >1.1 | | | 99.4 | >100 | |
| 146 | 0.12 | | | 112.5 | >100 | |
| 147 | 0.08 | | | | | |
| 148 | >1.1 | | | | | |
| 149 | 0.6 | | | | | |
| 150 | >1.1 | | | | | |
| 151 | >1.1 | | | 87.4 | >100 | |
| 152 | 0.026 | | | 82.7 | >100 | |
| 153 | 0.21 | | | 94.2 | >100 | |
| 154 | 0.013 | | | 73 | >100 | |
| 155 | 0.035 | | | 97.0 | >100 | |
| 156 | 0.083 | | | 106.9 | >100 | |
| 157 | 0.066 | | | | | |
| 158 | 0.23 | | | | | |
| 159 | >3.3 | 4 | | | | |
| 160 | 0.43 | | | | | |
| 161 | 0.074 | | | | | |
| 162 | 0.036 | | | | | |
| 163 | 0.007 | | | 99.0 | >100 | |
| 164 | >1.1 | | | 100.0 | >100 | |
| 165 | >1.1 | | | | | |
| 166 | >1.1 | | | | | |
| 167 | 0.19 | | | ND | ND | |
| 168 | 0.073 | | | 59.8 | >100 | 4.43 |
| 169 | >1.1 | | | | | |
| 170 | 0.3 | | | | | |
| 171 | 0.28 | | | | | |
| 172 | 0.24 | | | | >100 | 2.5 |
| 173 | >1.1 | | | | >100 | 2.0 |
| 174 | 0.48 | | | 96.2 | >100 | 2.42 |
| 175 | 0.041 | | | | | |
| 176 | 0.036 | | | 53.5 | >100 | |
| 177 | 0.1 | | | | | |
| 178 | 0.022 | | | 68.5 | >100 | |
| 179 | 0.7 | | | 79.3 | >100 | |
| 180 | 0.064 | | | 88.1 | >100 | |
| 181 | 0.13 | | | 64.9 | >100 | |
| 182 | >1.1 | | | 103.9 | >100 | |
| 183 | >1.1 | | | | | |
| 184 | 0.25 | | | | | |
| 185 | >1.1 | | | | | |
| 186 | >1.1 | | | 86.4 | >100 | |
| 187 | >1.1 | | | | >100 | 2.4 |
| 188 | 0.016 | | | 97.9 | >100 | 4.53 |
| 189 | 0.0065 | | | 58.0 | >100 | 3.98 |
| 190 | 0.02 | | | | | 2.90 |
| 191 | 0.024 | | | | | 3.45 |
| 192 | 0.059 | | | 104.0 | >100 | |
| 193 | 0.18 | | | | >100 | |
| 194 | 0.021 | | | | >100 | |
| 195 | >1.1 | | | 100.5 | >100 | |
| 196 | 0.16 | | | 29.2 | 76.4 | |
| 197 | 0.019 | | | 11.5 | 79.1 | |
| 198 | >1.1 | | | 102.2 | >100 | |
| 199 | 0.75 | | | 95.7 | >100 | |
| 200 | >1.1 | | | | | |
| 201 | 0.028 | | | 107.2 | >100 | |
| 202 | 0.046 | | | 82.3 | >100 | |
| 203 | 0.013 | | | | | |
| 204 | 0.036 | | | | | |
| 205 | >1.1 | | | | | |
| 206 | 0.18 | | | 89.6 | >100 | |
| 207 | 0.74 | | | 87.7 | >100 | |
| 208 | >1.1 | | | ND | ND | |
| 209 | 0.016 | | | | | |
| 210 | 0.13 | | | | | |
| 211 | 0.031 | | | | | |
| 212 | 0.21 | | | 99.4 | >100 | |
| 213 | 0.016 | | | 63.1 | >100 | |
| 214 | 0.22 | | | 76.8 | >100 | |
| 215 | 0.02 | | | 67.0 | >100 | |
| 216 | >1.1 | | | | | |
| 217 | >1.1 | | | | | |
| 218 | >1.1 | | | | | |
| 219 | 0.012 | | | 53.7 | >100 | |
| 220 | 0.048 | | | | | |
| 221 | >1.1 | | | | | |
| 222 | 0.12 | | | 2.5 | 31.7 | |
| 223 | 0.07 | | | 87.5 | >100 | |
| 224 | 0.21 | | | 100.0 | >100 | |
| 225 | 0.21 | | | 104.7 | >100 | |

*% inhibition rate (@3 μM)

Based on the above description, it will be understood by those skilled in the art that the present disclosure may be implemented in a different specific form without changing the technical spirit or essential characteristics thereof. Therefore, it should be understood that the above embodiments are not limitative, but illustrative in all aspects. The scope of the disclosure is defined by the appended claims rather than by the description preceding them, and therefore all changes and modifications that fall within metes and bounds of the claims or equivalents of such metes and bounds are intended to be embraced by the claims.

1. A compound represented by the following Chemical Formula 1 or a pharmaceutically acceptable salt of the compound: 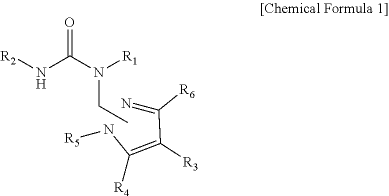
wherein:
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently a bond, hydrogen, cyano, halogen, C₁₋₄ alkyl, C₁₋₄ alkenyl, C₁₋₄ haloalkyl, C₁₋₄ hydroxyalkyl, C₁₋₄ alkoxy, C₁₋₃ acyl, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including carbon to which R₃ and R₄ are bonded;
Chemical Formula 1 is connected to a urea backbone through either of R₃ or R₆; and
a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, and 5- to 10-membered heteroaryl.

2. The compound according to claim 1 or a pharmaceutically acceptable salt of the compound, wherein R₁ is phenyl, bicyclo[1.1.1]pentyl, dihydropyridinyl, isoxazolyl, pyrazolyl, pyridazinyl, pyridinyl, pyrimidinyl, or thiadiazolyl.

3. The compound according to claim 1 or a pharmaceutically acceptable salt of the compound, wherein R₂ is phenyl, bicyclo[1.1.1]pentyl, imidazolyl, pyridinyl, or pyrimidinyl.

4. The compound according to claim 1 or a pharmaceutically acceptable salt of the compound, wherein R₃ is hydrogen, bromo, methyl, propenyl, trifluoromethyl, hydroxymethyl, hydroxyethyl, hydroxyisopropyl, hydroxypropyl, hydroxyisobutyl, methoxy, methoxyethyl, methoxymethyl, ethoxyethyl, ethoxyethenyl, or (4,4,5,5-tetramethyl-1,3-dioxolanyl)methyl.

5. The compound according to claim 1 or a pharmaceutically acceptable salt of the compound, wherein R₄ is a bond, hydrogen, cyano, methyl, ethyl, isopropyl, difluoromethyl, trifluoromethyl, or methoxy.

6. The compound according to claim 1 or a pharmaceutically acceptable salt of the compound, wherein R₃ and R₄ are connected to each other to form tetrahydrothiopyranyl, tetrahydropyranyl, tetrahydrofuranyl, piperidinyl, phenyl, dioxanyl, cyclopentyl, cyclohexyl, cycloheptyl, or bicyclo[2.2.1]heptyl including carbon to which R₃ and R₄ are bonded.

7. The compound according to claim 1 or a pharmaceutically acceptable salt of the compound, wherein R₅ is hydrogen, methyl, hydroxymethyl, hydroxyethyl, ethylcarbonyl, or tetrahydropyranyloxyethyl.

8. The compound according to claim 1 or a pharmaceutically acceptable salt of the compound, wherein R₆ is a bond or methyl.

9. The compound according to claim 1 or a pharmaceutically acceptable salt of the compound, wherein a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, bromo, fluoro, chloro, methyl, methylthio, methoxy, methoxymethyl, methylcarbonyl, methoxycarbonyl, methylsulfonylamino, methylaminosulfonyl, hydroxymethyl, hydroxyisopropyl, difluoromethyl, trifluoromethyl, methylamino, and dimethylamino.

10. The compound according to claim 1 or a pharmaceutically acceptable salt of the compound, wherein the compound is:
1. 3-(bicyclo[1.1.1]pentan-1-yl)-1-(2-methoxypyrimidin-5-yl)-1-((5-(trifluoromethyl)-1H-pyrazol-3-yl)methyl)urea,
2. 1-(2-methoxypyrimidin-5-yl)-3-(pyrimidin-5-yl)-1-((5-(trifluoromethyl)-1H-pyrazol-3-yl)methyl)urea,
3. 3-(3-fluoro-4-(1H-imidazol-1-yl)pyridin-2-yl)-1-(2-methoxypyrimidin-5-yl)-1-((5-(trifluoromethyl)-1H-pyrazol-3-yl)methyl)urea,
4. 3-(2-bromo-3-fluoropyridin-4-yl)-1-(2-methoxypyrimidin-5-yl)-1-((5-(trifluoromethyl)-1H-pyrazol-3-yl)methyl)urea,
5. 3-(6-chloropyridin-2-yl)-1-(2-methoxypyrimidin-5-yl)-1-((5-(trifluoromethyl)-1H-pyrazol-3-yl)methyl)urea,
6. 3-(3-chlorobicyclo[1.1.1]pentan-1-yl)-1-(2-methoxypyrimidin-5-yl)-1-((5-(trifluoromethyl)-1H-pyrazol-3-yl)methyl)urea,
7. 3-(3-chloro-4-cyanophenyl)-1-(2-methoxypyrimidin-5-yl)-1-((5-(trifluoromethyl)-1H-pyrazol-3-yl)methyl)urea,
8. 3-(5-chloro-6-cyanopyridin-2-yl)-1-(2-methoxypyrimidin-5-yl)-1-((5-(trifluoromethyl)-1H-pyrazol-3-yl)methyl)urea,
9. 3-(3-chloro-4-isocyanophenyl)-1-((5-cyano-1H-pyrazol-3-yl)methyl)-1-(2-methoxypyrimidin-5-yl)urea,
10. 3-(3-cyano-4,5-difluorophenyl)-1-((5-(difluoromethyl)-1H-pyrazol-3-yl)methyl)-1-(2-methoxypyrimidin-5-yl)urea,
11. 3-(3-chloro-4,5-difluorophenyl)-1-(2-methoxypyrimidin-5-yl)-1-((5-(trifluoromethyl)-1H-pyrazol-3-yl)methyl)urea,
12. 3-(3-chloro-4,5-difluorophenyl)-1-((5-(difluoromethyl)-1H-pyrazol-3-yl)methyl)-1-(2-methoxypyrimidin-5-yl)urea,
13. 3-(6-chloro-4,5-difluoropyridin-2-yl)-1-(2-methoxypyrimidin-5-yl)-1-((5-(trifluoromethyl)-1H-pyrazol-3-yl)methyl)urea,
14. 3-(3-chloro-4-fluorophenyl)-1-(6-oxo-1,6-dihydropyridin-3-yl)-1-((4,5,6,7-tetrahydro-1H-indazol-3-yl)methyl)urea,
15. 3-(3-chloro-4-fluorophenyl)-1-(2-oxo-1,2-dihydropyridin-4-yl)-1-((4,5,6,7-tetrahydro-1H-indazol-3-yl)methyl)urea,
16. N-(4-(3-(3-chloro-4-fluorophenyl)-1-((1,4,5,7-tetrahydropyrano[3,4-c]pyrazol-3-yl)methyl)ureido)phenyl)methanesulfonamide,
17. N-(5-(3-(3-chloro-4-fluorophenyl)-1-((1,4,5,7-tetrahydropyrano[3,4-c]pyrazol-3-yl)methyl)ureido)pyridin-2-yl)methanesulfonamide,
18. N-(5-(3-(3-chloro-4-fluorophenyl)-1-((1,4,5,6-tetrahydropyrano[2,3-c]pyrazol-3-yl)methyl)ureido)pyridin-2-yl)methanesulfonamide,
19. N-(4-(3-(3-chloro-4-fluorophenyl)-1-((1,4,5,6-tetrahydropyrano[2,3-c]pyrazol-3-yl)methyl)ureido)phenyl)methanesulfonamide,
20. 1-(6-acetylpyridin-3-yl)-3-(3-chloro-4-fluorophenyl)-1-((1,4,5,6-tetrahydropyrano[2,3-c]pyrazol-3-yl)methyl)urea,
21. 1-(6-acetylpyridin-3-yl)-3-(3-chloro-4-fluorophenyl)-1-((1,4,5,7-tetrahydropyrano[3,4-c]pyrazol-3-yl)methyl)urea,
22. 1-(2-acetylpyridin-4-yl)-3-(3-chloro-4-fluorophenyl)-1-((1,4,5,7-tetrahydropyrano[3,4-c]pyrazol-3-yl)methyl)urea,
23. 4-(3-(3-chloro-4-fluorophenyl)-1-((1,4,5,7-tetrahydropyrano[3,4-c]pyrazol-3-yl)methyl)ureido)-N-methylbenzenesulfonamide,
24. 3-(3-chloro-4-fluorophenyl)-1-(1-methyl-1H-pyrazol-4-yl)-1-((4,5,6,7-tetrahydro-1H-indazol-3-yl)methyl)urea,
25. 3-(3-chloro-4-fluorophenyl)-1-(1-methyl-1H-pyrazol-3-yl)-1-((1,4,5,6-tetrahydropyrano[2,3-c]pyrazol-3-yl)methyl)urea,
26. 3-(3-chloro-4-fluorophenyl)-1-(5-methylisoxazol-3-yl)-1-((1,4,5,6-tetrahydropyrano[2,3-c]pyrazol-3-yl)methyl)urea,
27. 3-(3-chloro-4-fluorophenyl)-1-(3-methylbicyclo[1.1.1]pentan-1-yl)-1-((5-(trifluoromethyl)-1H-pyrazol-3-yl)methyl)urea,
28. 3-(3-chloro-4-fluorophenyl)-1-(2-methoxy-4-methylpyrimidin-5-yl)-1-((5-(trifluoromethyl)-1H-pyrazol-3-yl)methyl)urea,
29. 3-(3-chloro-4-fluorophenyl)-1-(4-methoxy-2,6-dimethylphenyl)-1-((5-(trifluoromethyl)-1H-pyrazol-3-yl)methyl)urea,
30. 3-(3-chloro-4-fluorophenyl)-1-(2-methoxy-4,6-dimethylpyrimidin-5-yl)-1-((5-(trifluoromethyl)-1H-pyrazol-3-yl)methyl)urea,
31. methyl 4-(3-(3-chloro-4-fluorophenyl)-1-((4,5,6,7-tetrahydro-1H-indazol-3-yl)methyl)ureido)benzoate,
32. 3-(3-chloro-4-fluorophenyl)-1-(4-methoxyphenyl)-1-((4,5,6,7-tetrahydro-1H-indazol-3-yl)methyl)urea,
33. 1-((5-acetyl-4,5,6,7-tetrahydro-1H-pyrazolo[4,3-c]pyridin-3-yl)methyl)-3-(3-chloro-4-fluorophenyl)-1-(4-methoxyphenyl)urea,
34. 1-((1H-pyrazol-3-yl)methyl)-3-(3-chloro-4-fluorophenyl)-1-(4-methoxyphenyl)urea,
35. 3-(3-chloro-4-fluorophenyl)-1-(4-methoxyphenyl)-1-((1-methyl-1H-pyrazol-3-yl)methyl)urea,
36. 3-(3-chloro-4-fluorophenyl)-1-(4-methoxyphenyl)-1-((1-methyl-4,5,6,7-tetrahydro-1H-indazol-3-yl)methyl)urea,
37. 3-(3-chloro-4-fluorophenyl)-1-(4-methoxyphenyl)-1-((5-methyl-4,5,6,7-tetrahydro-1H-pyrazolo[4,3-c]pyridin-3-yl)methyl)urea,
38. 3-(3-chloro-4-fluorophenyl)-1-(4-methoxyphenyl)-1-((1,4,6,7-tetrahydropyrano[4,3-c]pyrazol-3-yl)methyl)urea,
39. 3-(3-chloro-4-fluorophenyl)-1-(4-methoxyphenyl)-1-((1-methyl-1,4,6,7-tetrahydropyrano[4,3-c]pyrazol-3-yl)methyl)urea,
40. 3-(3-chloro-4-fluorophenyl)-1-((5,5-difluoro-4,5,6,7-tetrahydro-1H-indazol-3-yl)methyl)-1-(6-methoxypyridin-3-yl)urea,
41. 3-(3-chloro-4-fluorophenyl)-1-(6-methoxypyridin-3-yl)-1-((1,4,5,6-tetrahydrocyclopenta[c]pyrazol-3-yl)methyl)urea,
42. 3-(3-chloro-4-fluorophenyl)-1-(6-methoxypyridin-3-yl)-1-((4,5,6,7-tetrahydro-1H-indazol-3-yl)methyl)urea,
43. 1-((1H-indazol-3-yl)methyl)-3-(3-chloro-4-fluorophenyl)-1-(6-methoxypyridin-3-yl)urea,
44. 3-(3-chloro-4-fluorophenyl)-1-((5-(hydroxymethyl)-4,5,6,7-tetrahydro-1H-indazol-3-yl)methyl)-1-(6-methoxypyridin-3-yl)urea,
45. 3-(3-chloro-4-fluorophenyl)-1-(6-methoxypyridin-3-yl)-1-((1,4,6,7-tetrahydrothiopyrano[4,3-c]pyrazol-3-yl)methyl)urea,
46. 3-(3-chloro-4-fluorophenyl)-1-(4-methoxyphenyl)-1-((1,4,5,6-tetrahydrocyclopenta[c]pyrazol-3-yl)methyl)urea,
47. 3-(3-chloro-4-fluorophenyl)-1-((5,5-dimethyl-4,5,6,7-tetrahydro-1H-indazol-3-yl)methyl)-1-(6-methoxypyridin-3-yl)urea,
48. 3-(3-chloro-4-fluorophenyl)-1-((5-hydroxy-4,5,6,7-tetrahydro-1H-indazol-3-yl)methyl)-1-(6-methoxypyridin-3-yl)urea,
49. 3-(3-chloro-4-fluorophenyl)-1-((5,5-dioxido-1,4,6,7-tetrahydrothiopyrano[4,3-c]pyrazol-3-yl)methyl)-1-(6-methoxypyridin-3-yl)urea,
50. 3-(3-chloro-4-fluorophenyl)-1-(6-methoxypyridin-3-yl)-1-((5-methyl-1H-pyrazol-3-yl)methyl)urea,
51. 3-(3-chloro-4-fluorophenyl)-1-(6-methoxypyridin-3-yl)-1-((4,5,6,7-tetrahydro-1H-4,7-methanoindazol-3-yl)methyl)urea,
52. 3-((3-(3-chloro-4-fluorophenyl)-1-(6-methoxypyridin-3-yl)ureido)methyl)-4,5,6,7-tetrahydro-1H-indazole-5-carboxylic acid,
53. methyl 3-((3-(3-chloro-4-fluorophenyl)-1-(6-methoxypyridin-3-yl)ureido)methyl)-4,5,6,7-tetrahydro-1H-indazole-5-carboxylate,
54. 3-(3-chloro-4-fluorophenyl)-1-(2-methoxypyridin-4-yl)-1-((4,5,6,7-tetrahydro-1H-indazol-3-yl)methyl)urea,
55. 3-(3-chloro-4-fluorophenyl)-1-(6-methoxypyridin-3-yl)-1-((1,4,6,7-tetrahydropyrano[4,3-c]pyrazol-3-yl)methyl)urea,
56. 3-(3-chloro-4-fluorophenyl)-1-(6-methoxypyridin-3-yl)-1-((4-methyl-1H-pyrazol-3-yl)methyl)urea,
57. 3-(3-chloro-4-fluorophenyl)-1-((4,5-dimethyl-1H-pyrazol-3-yl)methyl)-1-(6-methoxypyridin-3-yl)urea,
58. 3-(3-chloro-4-fluorophenyl)-1-((5-isopropyl-1H-pyrazol-3-yl)methyl)-1-(6-methoxypyridin-3-yl)urea,
59. 3-(3-chloro-4-fluorophenyl)-1-(6-methoxypyridin-3-yl)-1-((6-methyl-1,4,5,6-tetrahydrocyclopenta[c]pyrazol-3-yl)methyl)urea,
60. 3-(3-chloro-4-fluorophenyl)-1-((5-ethyl-1H-pyrazol-3-yl)methyl)-1-(6-methoxypyridin-3-yl)urea,
61. 1-((4-bromo-1H-pyrazol-3-yl)methyl)-3-(3-chloro-4-fluorophenyl)-1-(4-methoxyphenyl)urea,
62. 3-(3-chloro-4-fluorophenyl)-1-(6-methoxypyridin-3-yl)-1-((5-(trifluoromethyl)-4,5,6,7-tetrahydro-1H-indazol-3-yl)methyl)urea,
63. 3-(3-chloro-4-fluorophenyl)-1-(6-methoxypyridin-3-yl)-1-((1,4,5,6-tetrahydropyrano[2,3-c]pyrazol-3-yl)methyl)urea,
64. 3-(3-chloro-4-fluorophenyl)-1-((5-methoxy-1H-pyrazol-3-yl)methyl)-1-(6-methoxypyridin-3-yl)urea,
65. 3-(3-chloro-4-fluorophenyl)-1-(1-methyl-6-oxo-1,6-dihydropyridin-3-yl)-1-((1,4,5,6-tetrahydrocyclopenta[c]pyrazol-3-yl)methyl)urea,
66. 3-(3-chloro-4-fluorophenyl)-1-(6-methoxypyridin-3-yl)-1-((1-(2-(tetrahydro-2H-pyran-2-yloxy)ethyl)-1,4,5,6-tetrahydrocyclopenta[c]pyrazol-3-yl)methyl)urea,
67. 3-(3-chloro-4-fluorophenyl)-1-((1-(2-hydroxyethyl)-1,4,5,6-tetrahydrocyclopenta[c]pyrazol-3-yl)methyl)-1-(6-methoxypyridin-3-yl)urea,
68. 3-(3-chloro-4-fluorophenyl)-1-((1,3a,4,5,6,7a-hexahydropyrano[2,3-c]pyrazol-3-yl)methyl)-1-(2-methoxypyridin-4-yl)urea,
69. 3-(3-chloro-4-fluorophenyl)-1-((4,6-dihydro-1H-furo[3,4-c]pyrazol-3-yl)methyl)-1-(6-methoxypyridin-3-yl)urea,
70. 3-(3-chloro-4-fluorophenyl)-1-((4,6-dihydro-1H-furo[3,4-c]pyrazol-3-yl)methyl)-1-(2-methoxypyridin-4-yl)urea,
71. 3-(3-chloro-4-fluorophenyl)-1-((5,6-dihydro-1H-[1,4]dioxino[2,3-c]pyrazol-3-yl)methyl)-1-(2-methoxypyridin-4-yl)urea,
72. 3-(3-chloro-4-fluorophenyl)-1-((5-(difluoromethyl)-1H-pyrazol-3-yl)methyl)-1-(6-methoxypyridin-3-yl)urea,
73. 3-(3-chloro-4-fluorophenyl)-1-(2-methoxypyridin-4-yl)-1-((1,4,6,7-tetrahydropyrano[4,3-c]pyrazol-3-yl)methyl)urea,
74. 3-(3-chloro-4-fluorophenyl)-1-(2-methoxypyridin-4-yl)-1-((5-(trifluoromethyl)-1H-pyrazol-3-yl)methyl)urea,
75. 3-(3-chloro-4-fluorophenyl)-1-(5-methoxypyridin-3-yl)-1-((1,4,6,7-tetrahydropyrano[4,3-c]pyrazol-3-yl)methyl)urea,
76. 3-(3-chloro-4-fluorophenyl)-1-(5-methoxypyridin-3-yl)-1-((1,4,5,6-tetrahydropyrano[2,3-c]pyrazol-3-yl)methyl)urea,
77. 3-(3-chloro-4-fluorophenyl)-1-((5-(difluoromethyl)-1H-pyrazol-3-yl)methyl)-1-(5-methoxypyridin-3-yl)urea,
78. 3-(3-chloro-4-fluorophenyl)-1-(6-methoxypyridin-3-yl)-1-((4-methyl-5-(trifluoromethyl)-1H-pyrazol-3-yl)methyl)urea,
79. 3-(3-chloro-4-fluorophenyl)-1-((1-(hydroxymethyl)-5-(trifluoromethyl)-1H-pyrazol-3-yl)methyl)-1-(6-methoxypyridin-3-yl)urea,
80. 3-(3-chloro-4-fluorophenyl)-1-(2-methoxypyrimidin-5-yl)-1-((5-(trifluoromethyl)-1H-pyrazol-3-yl)methyl)urea,
81. 1-((4-bromo-5-(difluoromethyl)-1H-pyrazol-3-yl)methyl)-3-(3-chloro-4-fluorophenyl)-1-(2-methoxypyrimidin-5-yl)urea,
82. 3-(3-chloro-4-fluorophenyl)-1-((5-(difluoromethyl)-4-(methoxymethyl)-1H-pyrazol-3-yl)methyl)-1-(6-methoxypyridin-3-yl)urea,
83. 3-(3-chloro-4-fluorophenyl)-1-((4-(2-ethoxyvinyl)-5-(trifluoromethyl)-1H-pyrazol-3-yl)methyl)-1-(6-methoxypyridin-3-yl)urea,
84. 3-(3-chloro-4-fluorophenyl)-1-((5-(difluoromethyl)-1H-pyrazol-3-yl)methyl)-1-(2-methoxypyrimidin-5-yl)urea,
85. 3-(3-chloro-4-fluorophenyl)-1-((4-(2-ethoxyethyl)-5-(trifluoromethyl)-1H-pyrazol-3-yl)methyl)-1-(6-methoxypyridin-3-yl)urea,
86. 3-(3-chloro-4-fluorophenyl)-1-((4-(2-hydroxyethyl)-5-(trifluoromethyl)-1H-pyrazol-3-yl)methyl)-1-(6-methoxypyridin-3-yl)urea,
87. 3-(3-chloro-4-fluorophenyl)-1-(6-methoxypyridin-3-yl)-1-((4-((4,4,5,5-tetramethyl-1,3-dioxolan-2-yl)methyl)-5-(trifluoromethyl)-1H-pyrazol-3-yl)methyl)urea,
88. 3-(3-chloro-4-fluorophenyl)-1-((5-(difluoromethyl)-4-(2-methoxyethyl)-1H-pyrazol-3-yl)methyl)-1-(6-methoxypyridin-3-yl)urea,
89. 3-(4-chloro-5-fluoropyridin-2-yl)-1-(2-methoxypyrimidin-5-yl)-1-((5-(trifluoromethyl)-1H-pyrazol-3-yl)methyl)urea,
90. 3-(3-chloro-4-fluorophenyl)-1-((5-methoxy-4-(trifluoromethyl)-1H-pyrazol-3-yl)methyl)-1-(6-methoxypyridin-3-yl)urea,
91. 3-(3-chloro-4-fluorophenyl)-1-((4-(2-hydroxyethyl)-5-(trifluoromethyl)-1H-pyrazol-3-yl)methyl)-1-(2-methoxypyrimidin-5-yl)urea,
92. 3-(3-chloro-4-fluorophenyl)-1-((5-(difluoromethyl)-4-(2-hydroxypropan-2-yl)-1H-pyrazol-3-yl)methyl)-1-(2-methoxypyrimidin-5-yl)urea,
93. 3-(3-chloro-4-fluorophenyl)-1-((5-(difluoromethyl)-4-(prop-1-en-2-yl)-1H-pyrazol-3-yl)methyl)-1-(2-methoxypyrimidin-5-yl)urea,
94. 3-(3-chloro-4-fluorophenyl)-1-(6-methoxypyridazin-4-yl)-1-((5-(trifluoromethyl)-1H-pyrazol-3-yl)methyl)urea,
95. 3-(3-chloro-4-fluorophenyl)-1-((4-(2-hydroxypropyl)-5-(trifluoromethyl)-1H-pyrazol-3-yl)methyl)-1-(2-methoxypyrimidin-5-yl)urea,
96. 3-(3-chloro-4-fluorophenyl)-1-(6-methoxypyridazin-3-yl)-1-((5-(trifluoromethyl)-1H-pyrazol-3-yl)methyl)urea,
97. 3-(3-chloro-4-fluorophenyl)-1-((5-(difluoromethyl)-1H-pyrazol-3-yl)methyl)-1-(6-methoxypyridazin-3-yl)urea,
98. 1-((4-bromo-5-(difluoromethyl)-1H-pyrazol-3-yl)methyl)-3-(3-chloro-4-fluorophenyl)-1-(2-methoxypyrimidin-5-yl)urea,
99. 3-(3-chloro-4-fluorophenyl)-1-((5-(difluoromethyl)-1H-pyrazol-3-yl)methyl)-1-(6-methoxypyridazin-4-yl)urea,
100. 3-(3-chloro-4-fluorophenyl)-1-((5-(difluoromethyl)-4-methoxy-1H-pyrazol-3-yl)methyl)-1-(6-methoxypyridin-3-yl)urea,
101. 3-(3-chloro-4-fluorophenyl)-1-((4-(2-hydroxy-2-methylpropyl)-5-(trifluoromethyl)-1H-pyrazol-3-yl)methyl)-1-(2-methoxypyrimidin-5-yl)urea,
102. 3-(3-chloro-4-fluorophenyl)-1-((4-(2-hydroxypropan-2-yl)-5-(trifluoromethyl)-1H-pyrazol-3-yl)methyl)-1-(2-methoxypyrimidin-5-yl)urea,
103. 3-(3-chloro-4-fluorophenyl)-1-((5-(difluoromethyl)-4-(2-hydroxy-2-methylpropyl)-1H-pyrazol-3-yl)methyl)-1-(2-methoxypyrimidin-5-yl)urea,
104. 3-(3-chloro-4-fluorophenyl)-1-((5-cyano-1H-pyrazol-3-yl)methyl)-1-(2-methoxypyrimidin-5-yl)urea,
105. 3-(3-chloro-4-fluorophenyl)-1-(3-methoxybicyclo[1.1.1]pentan-1-yl)-1-((5-(trifluoromethyl)-1H-pyrazol-3-yl)methyl)urea,
106. 3-(6-chloro-5-fluoropyridin-2-yl)-1-(2-methoxypyrimidin-5-yl)-1-((5-(trifluoromethyl)-1H-pyrazol-3-yl)methyl)urea,
107. 3-(5-chloro-6-fluoropyridin-3-yl)-1-(2-methoxypyrimidin-5-yl)-1-((5-(trifluoromethyl)-1H-pyrazol-3-yl)methyl)urea,
108. 3-(3-chloro-4-fluorophenyl)-1-((1,3-dimethyl-1H-pyrazol-5-yl)methyl)-1-(4-methoxyphenyl)urea,
109. 3-(3-chloro-4-fluorophenyl)-1-(3-(2-hydroxypropan-2-yl)isoxazol-5-yl)-1-((1,4,5,6-tetrahydropyrano[2,3-c]pyrazol-3-yl)methyl)urea,
110. 3-(3-chloro-4-fluorophenyl)-1-(6-(2-hydroxypropan-2-yl)pyridin-3-yl)-1-((1,4,5,6-tetrahydropyrano[2,3-c]pyrazol-3-yl)methyl)urea,
111. 3-(3-chloro-4-fluorophenyl)-1-(6-(2-hydroxypropan-2-yl)pyridin-3-yl)-1-((1,4,5,7-tetrahydropyrano[3,4-c]pyrazol-3-yl)methyl)urea,
112. 3-(3-chloro-4-fluorophenyl)-1-(2-(2-hydroxypropan-2-yl)pyridin-4-yl)-1-((1,4,5,7-tetrahydropyrano[3,4-c]pyrazol-3-yl)methyl)urea,
113. 3-(3-chloro-4-fluorophenyl)-1-(2-(2-hydroxypropan-2-yl)pyridin-4-yl)-1-((1,4,5,6-tetrahydropyrano[2,3-c]pyrazol-3-yl)methyl)urea,
114. 3-(3-chloro-4-fluorophenyl)-1-(4-hydroxyphenyl)-1-((1,4,5,6-tetrahydrocyclopenta[c]pyrazol-3-yl)methyl)urea,
115. 3-(3-chloro-4-fluorophenyl)-1-(3-(2-hydroxypropan-2-yl)isoxazol-5-yl)-1-((1,4,5,7-tetrahydropyrano[3,4-c]pyrazol-3-yl)methyl)urea,
116. 3-(3-chloro-4-fluorophenyl)-1-(3,4-dimethoxyphenyl)-1-((4,5,6,7-tetrahydro-1H-indazol-3-yl)methyl)urea,
117. 3-(3-chloro-4-fluorophenyl)-1-(3,5-dimethoxyphenyl)-1-((4,5,6,7-tetrahydro-1H-indazol-3-yl)methyl)urea,
118. 3-(3-chloro-4-fluorophenyl)-1-(6-cyano-5-methoxypyridin-3-yl)-1-((5-(trifluoromethyl)-1H-pyrazol-3-yl)methyl)urea,
119. 3-(3-chloro-4-fluorophenyl)-1-(5-cyano-6-methoxypyridin-3-yl)-1-((5-(trifluoromethyl)-1H-pyrazol-3-yl)methyl)urea,
120. 3-(3-chloro-4-fluorophenyl)-1-(5-cyanopyridin-3-yl)-1-((1,4,5,6-tetrahydropyrano[2,3-c]pyrazol-3-yl)methyl)urea,
121. 3-(3-chloro-4-fluorophenyl)-1-(6-cyanopyridin-3-yl)-1-((1,4,6,7-tetrahydropyrano[4,3-c]pyrazol-3-yl)methyl)urea,
122. 3-(3-chloro-4-fluorophenyl)-1-(2-cyanopyridin-4-yl)-1-((1,4,5,6-tetrahydropyrano[2,3-c]pyrazol-3-yl)methyl)urea,
123. 3-(3-chloro-4-fluorophenyl)-1-(2-cyanopyridin-4-yl)-1-((1,4,6,7-tetrahydropyrano[4,3-c]pyrazol-3-yl)methyl)urea,
124. 3-(3-chloro-4-fluorophenyl)-1-(6-cyanopyridin-3-yl)-1-((1,4,5,6-tetrahydropyrano[2,3-c]pyrazol-3-yl)methyl)urea,
125. 3-(3-chloro-4-fluorophenyl)-1-(6-cyanopyridin-3-yl)-1-((1,4,6,7-tetrahydropyrano[4,3-c]pyrazol-3-yl)methyl)urea,
126. 3-(3-chloro-4-fluorophenyl)-1-(6-cyanopyridin-3-yl)-1-((5-(difluoromethyl)-1H-pyrazol-3-yl)methyl)urea,
127. 3-(3-chloro-4-fluorophenyl)-1-(6-cyanopyridin-3-yl)-1-((1,4,5,7-tetrahydropyrano[3,4-c]pyrazol-3-yl)methyl)urea,
128. 3-(3-chloro-4-fluorophenyl)-1-(2-cyanopyridin-4-yl)-1-((1,4,5,7-tetrahydropyrano[3,4-c]pyrazol-3-yl)methyl)urea,
129. 3-(3-chloro-4-fluorophenyl)-1-(5-cyanopyridin-3-yl)-1-((1,4,6,7-tetrahydropyrano[4,3-c]pyrazol-3-yl)methyl)urea,
130. 3-(3-chloro-4-fluorophenyl)-1-(6-cyanopyridin-3-yl)-1-((5-(trifluoromethyl)-1H-pyrazol-3-yl)methyl)urea,
131. 4-(3-(3-chloro-4-fluorophenyl)-1-((4,5,6,7-tetrahydro-1H-indazol-3-yl)methyl)ureido)benzoic acid,
132. 3-(3-chloro-4-fluorophenyl)-1-(isoxazol-3-yl)-1-((4,5,6,7-tetrahydro-1H-indazol-3-yl)methyl)urea,
133. 3-(3-chloro-4-fluorophenyl)-1-((4,5,6,7-tetrahydro-1H-indazol-3-yl)methyl)-1-(1,3,4-thiadiazol-2-yl)urea,
134. 3-(3-chloro-4-fluorophenyl)-1-(isoxazol-3-yl)-1-((1,4,5,6-tetrahydrocyclopenta[c]pyrazol-3-yl)methyl)urea,
135. 3-(3-chloro-4-fluorophenyl)-1-(1H-pyrazol-3-yl)-1-((1,4,5,6-tetrahydropyrano[2,3-c]pyrazol-3-yl)methyl)urea,
136. 3-(3-chloro-4-fluorophenyl)-1-(isoxazol-3-yl)-1-((1,4,5,6-tetrahydropyrano[2,3-c]pyrazol-3-yl)methyl)urea,
137. 1-(bicyclo[1.1.1]pentan-1-yl)-3-(3-chloro-4-fluorophenyl)-1-((5-(trifluoromethyl)-1H-pyrazol-3-yl)methyl)urea,
138. 3-(6-chloro-4-(dimethylamino)-5-fluoropyridin-2-yl)-1-(2-methoxypyrimidin-5-yl)-1-((5-(trifluoromethyl)-1H-pyrazol-3-yl)methyl)urea,
139. 3-(3-chloro-4-fluoro-5-methylphenyl)-1-((5-(difluoromethyl)-1H-pyrazol-3-yl)methyl)-1-(2-methoxypyrimidin-5-yl)urea,
140. 3-(3-chloro-4-fluoro-5-methylphenyl)-1-(2-methoxypyrimidin-5-yl)-1-((5-(trifluoromethyl)-1H-pyrazol-3-yl)methyl)urea,
141. 3-(6-chloro-3-fluoro-4-methylpyridin-2-yl)-1-(2-methoxypyrimidin-5-yl)-1-((5-(trifluoromethyl)-1H-pyrazol-3-yl)methyl)urea,
142. 3-(6-chloro-5-fluoro-4-methylpyridin-2-yl)-1-(2-methoxypyrimidin-5-yl)-1-((5-(trifluoromethyl)-1H-pyrazol-3-yl)methyl)urea,
143. 3-(2-chloropyrimidin-5-yl)-1-(2-methoxypyrimidin-5-yl)-1-((5-(trifluoromethyl)-1H-pyrazol-3-yl)methyl)urea,
144. 3-(3-cyanophenyl)-1-(2-methoxypyrimidin-5-yl)-1-((5-(trifluoromethyl)-1H-pyrazol-3-yl)methyl)urea,
145. 3-(6-cyanopyridin-2-yl)-1-(2-methoxypyrimidin-5-yl)-1-((5-(trifluoromethyl)-1H-pyrazol-3-yl)methyl)urea,
146. 3-(2-cyanopyridin-4-yl)-1-(2-methoxypyrimidin-5-yl)-1-((5-(trifluoromethyl)-1H-pyrazol-3-yl)methyl)urea,
147. 3-(3-cyanophenyl)-1-((5-(difluoromethyl)-1H-pyrazol-3-yl)methyl)-1-(2-methoxypyrimidin-5-yl)urea,
148. 3-(6-cyanopyridin-2-yl)-1-(2-methoxypyrimidin-5-yl)-1-((5-(trifluoromethyl)-1H-pyrazol-3-yl)methyl)urea,
149. 3-(3-cyano-4,5-difluorophenyl)-1-(2-methoxypyrimidin-5-yl)-1-((5-(trifluoromethyl)-1H-pyrazol-3-yl)methyl)urea,
150. 3-(3-cyano-4,5-difluorophenyl)-1-((5-(difluoromethyl)-1H-pyrazol-3-yl)methyl)-1-(2-methoxypyrimidin-5-yl)urea,
151. 3-(6-cyano-4,5-difluoropyridin-2-yl)-1-(2-methoxypyrimidin-5-yl)-1-((5-(trifluoromethyl)-1H-pyrazol-3-yl)methyl)urea,
152. 3-(4-cyano-3-(difluoromethyl)phenyl)-1-(2-methoxypyrimidin-5-yl)-1-((5-(trifluoromethyl)-1H-pyrazol-3-yl)methyl)urea,
153. 1-((5-cyano-1H-pyrazol-3-yl)methyl)-3-(4-cyano-3-(difluoromethyl)phenyl)-1-(2-methoxypyrimidin-5-yl)urea,
154. 3-(3-cyano-4-fluorophenyl)-1-(2-methoxypyrimidin-5-yl)-1-((5-(trifluoromethyl)-1H-pyrazol-3-yl)methyl)urea,
155. 3-(3-cyano-4-fluorophenyl)-1-((5-(difluoromethyl)-1H-pyrazol-3-yl)methyl)-1-(2-methoxypyrimidin-5-yl)urea,
156. 3-(4-cyano-3-fluorophenyl)-1-(2-methoxypyrimidin-5-yl)-1-((5-(trifluoromethyl)-1H-pyrazol-3-yl)methyl)urea,
157. 1-((5-cyano-1H-pyrazol-3-yl)methyl)-3-(3-cyano-4-fluorophenyl)-1-(2-methoxypyrimidin-5-yl)urea,
158. 3-(2-cyano-3-fluoropyridin-4-yl)-1-(2-methoxypyrimidin-5-yl)-1-((5-(trifluoromethyl)-1H-pyrazol-3-yl)methyl)urea,
159. 3-(3-chloro-4-fluorophenyl)-1-((1,3-dimethyl-1H-pyrazol-4-yl)methyl)-1-(4-methoxyphenyl)urea,
160. 3-(6-cyano-5-fluoropyridin-2-yl)-1-(2-methoxypyrimidin-5-yl)-1-((5-(trifluoromethyl)-1H-pyrazol-3-yl)methyl)urea,
161. 3-(4-cyano-5-fluoropyridin-2-yl)-1-(2-methoxypyrimidin-5-yl)-1-((5-(trifluoromethyl)-1H-pyrazol-3-yl)methyl)urea,
162. 3-(3-cyano-4-fluorophenyl)-1-(3-methoxybicyclo[1.1.1]pentan-1-yl)-1-((5-(trifluoromethyl)-1H-pyrazol-3-yl)methyl)urea,
163. 3-(3-cyano-4-fluorophenyl)-1-(6-methoxypyridazin-4-yl)-1-((5-(trifluoromethyl)-1H-pyrazol-3-yl)methyl)urea,
164. 3-(6-cyano-4-(dimethylamino)-5-fluoropyridin-2-yl)-1-(2-methoxypyrimidin-5-yl)-1-((5-(trifluoromethyl)-1H-pyrazol-3-yl)methyl)urea,
165. 3-(2,6-dichloropyridin-3-yl)-1-(2-methoxypyrimidin-5-yl)-1-((5-(trifluoromethyl)-1H-pyrazol-3-yl)methyl)urea,
166. 3-(2,6-dichloropyridin-3-yl)-1-((5-(difluoromethyl)-1H-pyrazol-3-yl)methyl)-1-(2-methoxypyrimidin-5-yl)urea,
167. 3-(3,4-dicyanophenyl)-1-(2-methoxypyrimidin-5-yl)-1-((5-(trifluoromethyl)-1H-pyrazol-3-yl)methyl)urea,
168. 3-(3,4-difluorophenyl)-1-(6-methoxypyridin-3-yl)-1-((5-(trifluoromethyl)-1H-pyrazol-3-yl)methyl)urea,
169. 3-(3,6-difluoropyridin-2-yl)-1-(2-methoxypyrimidin-5-yl)-1-((5-(trifluoromethyl)-1H-pyrazol-3-yl)methyl)urea,
170. 3-(5,6-difluoropyridin-2-yl)-1-(2-methoxypyrimidin-5-yl)-1-((5-(trifluoromethyl)-1H-pyrazol-3-yl)methyl)urea,
171. 3-(5,6-difluoropyridin-3-yl)-1-(2-methoxypyrimidin-5-yl)-1-((5-(trifluoromethyl)-1H-pyrazol-3-yl)methyl)urea,
172. 3-(4,5-difluoropyridin-2-yl)-1-(2-methoxypyrimidin-5-yl)-1-((5-(trifluoromethyl)-1H-pyrazol-3-yl)methyl)urea,
173. 3-(3,4-difluoropyridin-2-yl)-1-(2-methoxypyrimidin-5-yl)-1-((5-(trifluoromethyl)-1H-pyrazol-3-yl)methyl)urea,
174. 3-(5-(difluoromethyl)pyridin-3-yl)-1-(2-methoxypyrimidin-5-yl)-1-((5-(trifluoromethyl)-1H-pyrazol-3-yl)methyl)urea,
175. 3-(2-(difluoromethyl)pyridin-4-yl)-1-(2-methoxypyrimidin-5-yl)-1-((5-(trifluoromethyl)-1H-pyrazol-3-yl)methyl)urea,
176. 3-(3-(difluoromethyl)phenyl)-1-(2-methoxypyrimidin-5-yl)-1-((5-(trifluoromethyl)-1H-pyrazol-3-yl)methyl)urea,
177. 3-(6-(difluoromethyl)pyridin-2-yl)-1-(2-methoxypyrimidin-5-yl)-1-((5-(trifluoromethyl)-1H-pyrazol-3-yl)methyl)urea,
178. 1-((5-(difluoromethyl)-1H-pyrazol-3-yl)methyl)-3-(3-(difluoromethyl)phenyl)-1-(2-methoxypyrimidin-5-yl)urea,
179. 1-((5-(difluoromethyl)-1H-pyrazol-3-yl)methyl)-3-(6-(difluoromethyl)pyridin-2-yl)-1-(2-methoxypyrimidin-5-yl)urea,
180. 1-((5-cyano-1H-pyrazol-3-yl)methyl)-3-(3-(difluoromethyl)phenyl)-1-(2-methoxypyrimidin-5-yl)urea,
181. 3-(3-(difluoromethyl)phenyl)-1-(3-methoxybicyclo[1.1.1]pentan-1-yl)-1-((5-(trifluoromethyl)-1H-pyrazol-3-yl)methyl)urea,
182. 1-((4-(2-ethoxyethyl)-5-(trifluoromethyl)-1H-pyrazol-3-yl)methyl)-3-(4-fluorophenyl)-1-(6-methoxypyridin-3-yl)urea,
183. 3-(6-fluoropyridin-2-yl)-1-(2-methoxypyrimidin-5-yl)-1-((5-(trifluoromethyl)-1H-pyrazol-3-yl)methyl)urea,
184. 3-(6-fluoropyridin-3-yl)-1-(2-methoxypyrimidin-5-yl)-1-((5-(trifluoromethyl)-1H-pyrazol-3-yl)methyl)urea,
185. 3-(3-fluorobicyclo[1.1.1]pentan-1-yl)-1-(2-methoxypyrimidin-5-yl)-1-((5-(trifluoromethyl)-1H-pyrazol-3-yl)methyl)urea,
186. 3-(2-fluoropyrimidin-5-yl)-1-(2-methoxypyrimidin-5-yl)-1-((5-(trifluoromethyl)-1H-pyrazol-3-yl)methyl)urea,
187. 3-(4-fluoropyridin-2-yl)-1-(2-methoxypyrimidin-5-yl)-1-((5-(trifluoromethyl)-1H-pyrazol-3-yl)methyl)urea,
188. 3-(3-(difluoromethyl)-4-fluorophenyl)-1-(6-methoxypyridin-3-yl)-1-((5-(trifluoromethyl)-1H-pyrazol-3-yl)methyl)urea,
189. 3-(3-(difluoromethyl)-4-fluorophenyl)-1-(2-methoxypyrimidin-5-yl)-1-((5-(trifluoromethyl)-1H-pyrazol-3-yl)methyl)urea,
190. 1-((5-(difluoromethyl)-1H-pyrazol-3-yl)methyl)-3-(3-(difluoromethyl)-4-fluorophenyl)-1-(2-methoxypyrimidin-5-yl)urea,
191. 1-((5-(difluoromethyl)-1H-pyrazol-3-yl)methyl)-3-(3-(difluoromethyl)-4-fluorophenyl)-1-(6-methoxypyridin-3-yl)urea,
192. 3-(4-(difluoromethyl)-5-fluoropyridin-2-yl)-1-(2-methoxypyrimidin-5-yl)-1-((5-(trifluoromethyl)-1H-pyrazol-3-yl)methyl)urea,
193. 3-(3-(difluoromethyl)-4-fluorophenyl)-1-((4-(2-hydroxyethyl)-5-(trifluoromethyl)-1H-pyrazol-3-yl)methyl)-1-(2-methoxypyrimidin-5-yl)urea,
194. 1-((4-bromo-5-(trifluoromethyl)-1H-pyrazol-3-yl)methyl)-3-(3-(difluoromethyl)-4-fluorophenyl)-1-(2-methoxypyrimidin-5-yl)urea,
195. 1-((5-(difluoromethyl)-1H-pyrazol-3-yl)methyl)-3-(3-(difluoromethyl)-4-fluorophenyl)-1-(6-methoxypyridazin-3-yl)urea,
196. 1-((5-(difluoromethyl)-1H-pyrazol-3-yl)methyl)-3-(6-(difluoromethyl)-5-fluoropyridin-2-yl)-1-(2-methoxypyrimidin-5-yl)urea,
197. 1-((4-bromo-5-(difluoromethyl)-1H-pyrazol-3-yl)methyl)-3-(3-(difluoromethyl)-4-fluorophenyl)-1-(2-methoxypyrimidin-5-yl)urea,
198. 1-((5-(difluoromethyl)-4-(2-hydroxypropan-2-yl)-1H-pyrazol-3-yl)methyl)-3-(3-(difluoromethyl)-4-fluorophenyl)-1-(2-methoxypyrimidin-5-yl)urea,
199. 3-(3-(difluoromethyl)-4-fluorophenyl)-1-((4-(2-hydroxypropyl)-5-(trifluoromethyl)-1H-pyrazol-3-yl)methyl)-1-(2-methoxypyrimidin-5-yl)urea,
200. 3-(3-(difluoromethyl)-4-fluorophenyl)-1-(6-methoxypyridazin-3-yl)-1-((5-(trifluoromethyl)-1H-pyrazol-3-yl)methyl)urea,
201. 1-((5-(difluoromethyl)-1H-pyrazol-3-yl)methyl)-3-(3-(difluoromethyl)-4-fluorophenyl)-1-(6-methoxypyridazin-4-yl)urea,
202. 3-(6-(difluoromethyl)-5-fluoropyridin-2-yl)-1-(2-methoxypyrimidin-5-yl)-1-((5-(trifluoromethyl)-1H-pyrazol-3-yl)methyl)urea,
203. 3-(3-(difluoromethyl)-4-fluorophenyl)-1-(6-methoxypyridazin-4-yl)-1-((5-(trifluoromethyl)-1H-pyrazol-3-yl)methyl)urea,
204. 3-(6-(difluoromethyl)-5-fluoropyridin-2-yl)-1-(2-methoxypyrimidin-5-yl)-1-((5-(trifluoromethyl)-1H-pyrazol-3-yl)methyl)urea,
205. 3-(3-(difluoromethyl)-4-fluorophenyl)-1-((4-(2-hydroxy-2-methylpropyl)-5-(trifluoromethyl)-1H-pyrazol-3-yl)methyl)-1-(2-methoxypyrimidin-5-yl)urea,
206. 3-(4-(difluoromethyl)-3-fluorophenyl)-1-(2-methoxypyrimidin-5-yl)-1-((5-(trifluoromethyl)-1H-pyrazol-3-yl)methyl)urea,
207. 3-(3-(difluoromethyl)-4-fluorophenyl)-1-((4-(2-hydroxypropan-2-yl)-5-(trifluoromethyl)-1H-pyrazol-3-yl)methyl)-1-(2-methoxypyrimidin-5-yl)urea,
208. 1-((5-(difluoromethyl)-4-(2-hydroxy-2-methylpropyl)-1H-pyrazol-3-yl)methyl)-3-(3-(difluoromethyl)-4-fluorophenyl)-1-(2-methoxypyrimidin-5-yl)urea,
209. 1-((5-cyano-1H-pyrazol-3-yl)methyl)-3-(3-(difluoromethyl)-4-fluorophenyl)-1-(2-methoxypyrimidin-5-yl)urea,
210. 1-((5-(difluoromethyl)-1H-pyrazol-3-yl)methyl)-3-(6-(difluoromethyl)-5-fluoropyridin-2-yl)-1-(2-methoxypyrimidin-5-yl)urea,
211. 3-(2-(difluoromethyl)-3-fluoropyridin-4-yl)-1-(2-methoxypyrimidin-5-yl)-1-((5-(trifluoromethyl)-1H-pyrazol-3-yl)methyl)urea,
212. 1-((5-cyano-1H-pyrazol-3-yl)methyl)-3-(6-(difluoromethyl)-5-fluoropyridin-2-yl)-1-(2-methoxypyrimidin-5-yl)urea,
213. 3-(3-(difluoromethyl)-4-fluorophenyl)-1-(3-methoxybicyclo[1.1.1]pentan-1-yl)-1-((5-(trifluoromethyl)-1H-pyrazol-3-yl)methyl)urea,
214. 3-(6-(difluoromethyl)-5-fluoropyridin-2-yl)-1-(3-methoxybicyclo[1.1.1]pentan-1-yl)-1-((5-(trifluoromethyl)-1H-pyrazol-3-yl)methyl)urea,
215. 1-((5-cyano-1H-pyrazol-3-yl)methyl)-3-(4-fluoro-3-(trifluoromethyl)phenyl)-1-(2-methoxypyrimidin-5-yl)urea,
216. 1,3-bis(2-methoxypyrimidin-5-yl)-1-((5-(trifluoromethyl)-1H-pyrazol-3-yl)methyl)urea,
217. 1-(2-methoxypyrimidin-5-yl)-3-(3-methylbicyclo[1.1.1]pentan-1-yl)-1-((5-(trifluoromethyl)-1H-pyrazol-3-yl)methyl)urea,
218. 1-(2-methoxypyrimidin-5-yl)-3-(2-(methylthio)pyrimidin-5-yl)-1-((5-(trifluoromethyl)-1H-pyrazol-3-yl)methyl)urea,
219. 1-(2-methoxypyrimidin-5-yl)-1-((5-(trifluoromethyl)-1H-pyrazol-3-yl)methyl)-3-(3,4,5-trifluorophenyl)urea,
220. 1-((5-(difluoromethyl)-1H-pyrazol-3-yl)methyl)-1-(2-methoxypyrimidin-5-yl)-3-(3,4,5-trifluorophenyl)urea,
221. 1-(2-methoxypyrimidin-5-yl)-1-((5-(trifluoromethyl)-1H-pyrazol-3-yl)methyl)-3-(3,5,6-trifluoropyridin-2-yl)urea,
222. 1-(3-methoxybicyclo[1.1.1]pentan-1-yl)-1-((5-(trifluoromethyl)-1H-pyrazol-3-yl)methyl)-3-(3,4,5-trifluorophenyl)urea,
223. 1-((5-cyano-1H-pyrazol-3-yl)methyl)-1-(2-methoxypyrimidin-5-yl)-3-(3,4,5-trifluorophenyl)urea,
224. 1-(2-methoxypyrimidin-5-yl)-1-((5-(trifluoromethyl)-1H-pyrazol-3-yl)methyl)-3-(4,5,6-trifluoropyridin-2-yl)urea,
225. 1-(2-methoxypyrimidin-5-yl)-1-((5-(trifluoromethyl)-1H-pyrazol-3-yl)methyl)-3-(6-(trifluoromethyl)pyridin-2-yl)urea, or
226. 3-(3-cyano-4-fluorophenyl)-1-(2-methoxypyrimidin-5-yl)-1-((1-propionyl-5-(trifluoromethyl)-1H-pyrazol-3-yl)methyl)urea.

11. A composition for capsid assembly inhibition, comprising the compound according to claim 1 or a pharmaceutically acceptable salt of the compound.

12. An antiviral composition comprising the compound according to claim 1 or a pharmaceutically acceptable salt of the compound as an active ingredient.

13. A pharmaceutical composition for prevention or treatment of viral disease, comprising the compound according to claim 1 or a pharmaceutically acceptable salt of the compound as an active ingredient.

14. The pharmaceutical composition according to claim 13, wherein the viral disease is an infectious disease caused by hepatitis B virus (HBV), hepatitis C virus (HCV), or human immunodeficiency virus (HIV).


SEQUENCE LISTING (3 sequences)

SEQ ID NO: 1 - DNA, 19 bp, synthetic construct
  source 1..19: mol_type = other DNA
ctcgtggtggacttctctc

SEQ ID NO: 2 - DNA, 18 bp, synthetic construct
  source 1..18: mol_type = other DNA
ctgcaggatgaagaggaa

SEQ ID NO: 3 - DNA, 26 bp, synthetic construct
  source 1..26: mol_type = other DNA
  misc_feature <1: note = 5' 56-FAM
  misc_feature 9..10: note = internal quencher ZEN
  misc_feature >26: note = 3' quencher 3IABkFQ
tgtcctggttatcgctggatgtgtct